(19) United States
(12) Patent Application Publication
(10) Pub. No.: US 2025/0230510 A1
(43) Pub. Date: Jul. 17, 2025

(54) METHODS FOR IDENTIFYING MICROBIAL STRAINS HAVING ENHANCED PLANT COLONIZATION EFFICIENCY

(71) Applicant: NewLeaf Symbiotics, Inc., St. Louis, MO (US)

(72) Inventors: Janne Kerovuo, Los Angeles, CA (US); Doug Bryant, Temecula, CA (US); Patrick Vogan, Shrewsbury, MO (US); Natalie Breakfield, St. Louis, MO (US)

(21) Appl. No.: 19/020,764

(22) Filed: Jan. 14, 2025

Related U.S. Application Data
(63) Continuation of application No. 17/310,450, filed on Aug. 4, 2021.
(60) Provisional application No. 62/802,831, filed on Feb. 8, 2019.
(60) Provisional application No. 62/802,805, filed on Feb. 8, 2019.

(51) Int. Cl. C12Q 1/689; A01H 17/00; A01N 63/20; A01N 63/30; C12Q 1/04; C12R 1/01

(52) U.S. Cl. CPC C12Q 1/689; C12Q 1/04; A01H 17/00; A01N 63/20; A01N 63/30; C12R 2001/01

(57) ABSTRACT

Methods of evaluating microorganisms to determine efficiency of colonization of a plant or plant part are described. Methods of identifying genetic elements correlated with colonization efficiency of plant-associated microorganism are also provided. Methods to identify microorganisms for use as inoculants for improved plant yield using the colonization screening methods or the presence of genetic elements associated with colonization efficiency are described. Further methods useful for identification of microorganisms useful as inoculants for improving plant yield are presented.

### CROSS-REFERENCE TO RELATED APPLICATIONS

This patent application is a continuation of U.S. Ser. No. 17/310,450, filed Aug. 4, 2021, which is a U.S. national stage of PCT/US2020/012041, filed Jan. 2, 2020, which claims the benefit of U.S. 62/802,805, filed Feb. 8, 2019, and U.S. 62/802,831, filed Feb. 8, 2019, each of which are incorporated herein by reference in their entireties.

### SEQUENCE LISTING STATEMENT

A sequence listing containing the XML file named “P13540US03_SequenceListing.xml” which is 559,072 bytes (measured in MS-Windows®) and created on Jan. 14, 2025, contains 90 protein sequences, and 45 nucleic acid sequences is provided herewith via the USPTO's EFS system, and is incorporated herein by reference in its entirety.

### BACKGROUND

Microbial colonization of plant host cells or tissues is an aspect in both beneficial and detrimental interactions between plant-associated microorganisms and their host. Beneficial interactions include, for example, plant growth promotion by colonizing bacteria or fungi, biopesticidal activity of colonizing microorganisms that protect the host plant from pathogens, and activities of colonizing microorganisms that increase yield of a plant. Detrimental interactions include, for example, colonization of plants by disease causing pathogens. Identification of microorganisms that efficiently colonize plants, plant parts or tissues is useful for development of microbial inoculants for improving plant cultivation. Identification of genes correlated with the ability of a microorganism to colonize a plant host cell or tissue will expand the understanding of plant host/microorganism interactions and allow for manipulation of these interactions to promote interactions that benefit the plant and disrupt interactions that are detrimental to plant growth.

### SUMMARY

Methods for identifying one or more genetic elements correlated with colonization efficiency of a plant-associated microorganism that comprise: (i) screening a population of plant-associated microorganisms to determine the ability of strains in said population to colonize a plant or plant part; (ii) identifying a first set of strains in said population that colonize said plant or plant part at an enhanced density as compared to a non-colonizing control treatment or other strains of said population; (iii) identifying a second set of strains in said population that colonize said plant or plant part at a reduced density as compared to other strains of said population, or at a density that is reduced or not significantly different from that of a non-colonizing control treatment; (iv) comparing the sequences of genetic elements in said first set of strains and said second set of strains; and (v) identifying one or more genetic elements that correlate with colonization efficiency.

Also provided are methods for selecting a microbial strain capable of efficiently colonizing a plant, plant cell or plant part that comprise detecting in the genome of said microbial strain one or more genetic elements that are positively correlated with colonization efficiency.

Further, methods are provided of selecting a microbial strain capable of efficiently colonizing a plant, plant cell, or plant part that comprise detecting the presence of one or more genetic elements in the genome of said microbial strain, wherein said one or more genetic elements (i) comprise a gene selected from the group consisting of bbsG, hdhA, luxQ, bicA, hddC, hddA, fptA, livF, sutR, cdhR, amaB, ssuA, rbn, ftsY, fecA, gpmA_2, ecfG_1, adh, lgt, yfih, cyaA, vgb_3, pimB_2, bmr3_2, and fabD_1, or (ii) encode at least one protein having an amino acid sequence of SEQ ID NO: 1 to SEQ ID NO: 45.

Also provided are methods of identifying a yield-enhancing plant-associated microorganism that comprise: (i) screening a population of plant-associated microorganisms to identify strains having tolerance to desiccation and tolerance to contact with agricultural chemicals, (ii) screening said population of plant-associated microorganisms to determine the ability of strains in said population to colonize a plant or plant part; (iii) identifying strains having tolerance to desiccation and contact with agricultural chemicals and strains in said population that colonize said plant or plant part at an enhanced density as compared to a non-colonizing control treatment; (iii) screening said population of plant-associated microorganisms or said strains identified as having tolerance to desiccation and agricultural chemicals and as having the ability to colonize a plant or plant part to identify strains that efficiently colonize a target plant host; (iv) contacting strains identified as having tolerance to desiccation and agricultural chemicals and that efficiently colonize a plant host with said plant host or a part of said plant host; (v) growing said plant host under field conditions; (vi) determining harvested yield of said plant host; and (vii) identifying strains that improve yield of said plant host.

### DETAILED DESCRIPTION

Methods for identification of plant-associated microorganisms that are capable of efficiently colonizing a plant host are provided and used to identify genetic elements that are correlated with enhanced colonization efficiency.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Where a term is provided in the singular, embodiments comprising the plural of that term are also provided.

As used herein, the terms “include,” “includes,” and “including” are to be construed as at least having the features or encompassing the items to which they refer while not excluding any additional unspecified features or unspecified items.

As used herein a “plant-associated microorganism” is a bacterium or fungus that is present in or lives on plants or in soil where plants are grown. Plant-associated microorganisms may be present in the rhizosphere or phyllosphere. Plant-associated microorganisms may also be endophytes that live within a plant or plant part.

As used herein, the term “desiccation tolerance” is intended to indicate the ability of a plant-associated microorganism to survive under conditions of extreme dryness.

As used herein, the term “strain” refers to a pure culture of a subject plant-associated microorganism as well as to the progeny or potential progeny of the subject microorganism. The term strain shall include all isolates of such strain.

As used herein, a “pan-genome” is the entire set of genes for the microbial population being screened in a plant colonization efficiency screen. Thus, a pan-genome may represent the entire set of genes for a particular species, or the entire set of genes in multiple different species of the same genus or even the entire set of genes for multiple species classified in more than a single genus, where the strains in the population are from closely related genera.

As used herein a “genetic element” refers to an element in a DNA or RNA molecule that comprises a series of adjacent nucleotides at least 20 nucleotides in length and up to 50, 100, 1000, or 10000 or more, nucleic acids in length. A genetic element may comprise different groups of adjacent nucleic acids, for example, where the genome of a plant-associated microorganism contains introns and exons. The genetic element may be present on a chromosome or on an extrachromosomal element, such as a plasmid. In eukaryotic plant-associated microorganisms, the genetic element may be present in the nucleus or in the mitochondria. In some embodiments, the genetic element is a functional genetic element (e.g., a gene) that encodes a protein.

As used herein, the terms “homologous” or “homologue” or “ortholog” refer to related genetic elements or proteins encoded by the genetic elements that are determined based on the degree of sequence identity. These terms describe the relationship between a genetic element or encoded protein found in one isolate, species or strain and the corresponding or equivalent genetic element or protein in another isolate, species or strain. As used herein, a particular genetic element in a first isolate, species or strain is considered equivalent to a genetic element present in a second isolate, species or strain when the proteins encoded by the genetic element in the isolates, species or strains have at least 50 percent identity. Percent identity can be determined using a number of software programs available in the art including BLASTP, ClustalW, ALLALIGN, DNASTAR, SIM, SEQALN, NEEDLE, SSEARCH and the like.

As used herein, the term “colonize” refers to the ability of a microorganism to grow and reproduce in an environment. A microorganism is considered to colonize a plant or plant part if it can survive and grow on or inside the plant or plant part, including inside a plant cell.

As used herein, a “population of plant-associated microorganisms” refers to a group of 2 or more strains of genetically related microorganisms. The genetically related microorganisms may, for example, be of the same genus, or of the same species.

“Colonization efficiency” as used herein refers to the relative ability of a given microbial strain to colonize a plant host cell or tissue as compared to non-colonizing control samples or other microbial strains. Colonization efficiency can be assessed, for example and without limitation, by determining colonization density, reported for example as colony forming units (CFU) per mg of plant tissue, or by quantification of nucleic acids specific for a strain in a colonization screen, for example using qPCR.

As used herein, a “non-colonizing control treatment” is a treatment that does not contain a strain of a plant-associated microorganism or contains a strain of a plant-associated microorganism that has previously been determined to exhibit low colonization efficiency or no ability to colonize a subject plant or plant part.

As used herein, a “correlation” is a statistical measure that indicates the extent to which two or more variables, here colonization efficiency and identified genetic elements, occur together. A positive correlation indicates that a microbial strain containing a given genetic element is likely to be an efficient colonizer. A negative correlation indicates that a microbial strain containing a given genetic element is likely to be a poor or inefficient colonizer.

As used herein, the term “Methylobacterium” refers to genera and species in the methylobacteriaceae family, including bacterial species in the Methylobacterium genus and proposed Methylorubrum genus (Green and Ardley (2018)). Methylobacterium includes pink-pigmented facultative methylotrophic bacteria (PPFM) and also encompasses the non-pink-pigmented Methylobacterium nodulans, as well as colorless mutants of Methylobacterium isolates. For example, and not by way of limitation, “Methylobacterium” refers to bacteria of the species listed below as well as any new Methylobacterium species that have not yet been reported or described that can be characterized as Methylobacterium or Methylorubrum based on phylogenetic analysis: Methylobacterium adhaesivum; Methylobacterium oryzae; Methylobacterium aerolatum; Methylobacterium oxalidis; Methylobacterium aquaticum; Methylobacterium persicinum; Methylobacterium brachiatum; Methylobacterium phyllosphaerae; Methylobacterium brachythecii; Methylobacterium phyllostachyos; Methylobacterium bullatum; Methylobacterium platani; Methylobacterium cerastii; Methylobacterium pseudosasicola; Methylobacterium currus; Methylobacterium radiotolerans; Methylobacterium dankookense; Methylobacterium soli; Methylobacterium frigidaeris; Methylobacterium specialis; Methylobacterium fujisawaense; Methylobacterium tardum; Methylobacterium gnaphalii; Methylobacterium tarhaniae; Methylobacterium goesingense; Methylobacterium thuringiense; Methylobacterium gossipiicola; Methylobacterium trifolii; Methylobacterium gregans; Methylobacterium variabile; Methylobacterium haplocladii; Methylobacterium aminovorans (Methylorubrum aminovorans); Methylobacterium hispanicum; Methylobacterium extorquens (Methylorubrum extorquens); Methylobacterium indicum; Methylobacterium podarium (Methylorubrum podarium); Methylobacterium iners; Methylobacterium populi (Methylorubrum populi); Methylobacterium isbiliense; Methylobacterium pseudosasae (Methylorubrum pseudosasae); Methylobacterium jeotgali; Methylobacterium rhodesianum (Methylorubrum rhodesianum); Methylobacterium komagatae; Methylobacterium rhodinum (Methylorubrum rhodinum); Methylobacterium longum; Methylobacterium salsuginis (Methylorubrum salsuginis); Methylobacterium marchantiae; Methylobacterium suomiense (Methylorubrum suomiense; Methylobacterium mesophilicum; Methylobacterium thiocyanatum (Methylorubrum thiocyanatum); Methylobacterium nodulans; Methylobacterium zatmanii (Methylorubrum zatmanii); Methylobacterium organophilum.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

Methods for identification of plant-associated microorganisms that are capable of efficiently colonizing a plant host are provided. In some embodiments, such methods are used to identify genetic elements in said plant-associated microorganisms that are correlated with enhanced colonization efficiency.

In some embodiments, the plant-associated microorganism is beneficial to a plant host, and the genetic elements correlated with enhanced colonization efficiency can be used to identify other microbial strains that comprise one or more of the genetic elements correlated with enhanced colonization of a plant host. In some embodiments, the plant beneficial microbial strains identified as being able to efficiently colonize a plant host are further screened for additional traits that can contribute the fitness of the plant-associated microbial strain for use as an agricultural inoculant. In some embodiments a population of plant-associated microorganisms is screened, prior to conducting a colonization efficiency screen, for additional traits that can contribute to the fitness of the plant-associated microbial strain for use as an agricultural inoculant. Additional screens that can be used to evaluate the fitness of a particular plant-associated microorganism for use as an agricultural inoculant are also provided and include screens for tolerance to desiccation, tolerance to agricultural chemicals, and screens for growth rate and ease of production when grown in media with varying sources of carbon, nitrogen and other nutrients, such as vitamins or other trace elements.

In some embodiments, the plant-associated microorganism is detrimental to a plant host, or to a human or animal that consumes the plant or food or feed prepared from said plant that comprises the detrimental plant-associated microorganism. In such embodiments, identified genetic elements correlated with colonization efficiency are used to develop methods to disrupt the function of the detrimental plant-associated microorganisms or prevent such detrimental plant-associated microorganisms from colonizing the plant host. Colonization efficiency screens are provided for determining the ability of a plant-associated microorganism to colonize a host plant or host plant part. In some embodiments, the plant-associated microorganism is beneficial to a plant, for example as a biostimulant, that improves yield, or a biopesticide that provides protection against plant pests. Microbial biostimulants benefit plants by enhancing nutrition efficiency, abiotic stress tolerance and/or crop quality traits, and include biofertilizers which increase the supply or availability of nutrients for the plant host. Thus, microbial biostimulants include mycorrhizal and non-mycorrhizal fungi, bacterial endosymbionts and rhizobacteria. Non-limiting example of plant-associated bacteria that can be used in the methods provided herein include species of Actinomycetes, Agrobacterium, Arthrobacter, Alcaligenes, Aureobacterium, Azobacter, Azorhizobium, Azospirillum, Azotobacter, Beijerinckia, Brevibacillus, Burkholderia, Chromobacterium, Clostridium, Clavibacter, Comomonas, Corynebacterium, Curtobacterium, Enterobacter, Flavobacterium, Gluconacetobacter, Gluconobacter, Herbaspirillum, Hydrogenophage, Klebsiella, Luteibacter, Lysinibacillus, Mesorhizobium, Methylobacterium, Microbacterium, Ochrobactrum, Paenibacillus, Pantoea, Pasteuria, Phingobacterium, Photorhabdus, Phyllobacterium, Pseudomonas, Rhizobium, Rhodococcus, Bradyrhizobium, Serratia, Sinorhizobium, Sphingomonas, Streptomyces, Stenotrophomonas, Variovorax, Xanthomonas and Xenorhadbus. Non-limiting example of plant-associated mycorrhizal and non-mycorrhizal fungi that can be used in the methods provided herein include species of Acremonium, Alternaria, Ampelomyces, Aspergillus, Aureobasidium, Beauveria, Botryosphaeria, Cladosporium, Cochliobolus, Colletotrichum, Coniothyrium, Embellisia, Epicoccum, Fusarium, Gigaspora, Gliocladium, Glomus, Laccaria, Metarhisium, Muscodor, Nigrospora, Paecilonyces, Paraglomus, Penicillium, Phoma, Pisolithus, Podospora, Rhizopogon, Scleroderma, Trichoderma, Typhula, Ulocladium, and Verticilium.

In some embodiments, a plant-associated microorganism in a colonization efficiency screen is detrimental to a plant host, for example as a plant pathogen. Plant pathogenic microorganisms include for example, plant pathogenic fungi, such as species of Fusarium, Colletotrichum, Septoria, Blumeria, Alternaria, Stagonospora, Stenocarpella, Pythium, Rhizoctonia, Magnaportha, Pyrenophora, Peronospora, Microdochium, Sclerotinia, Sclerospora, Sclerophthora, Phytophthora, Cercospora, Gibberella, Verticillium and Pythium; and plant pathogenic bacteria, including species of Xanthomonas, Ralstonia, Erwinia, Pseudomonas, Streptomyces, Corynebacterium, Clavibacter, Xylella, Pectobacterium, Dickeya, Pantoea, Serratia, and Sphingomonas. In some embodiments, a plant-associated microorganism is detrimental to humans or other animals that consume a plant part that has been colonized by the microorganism. Plant-associated microorganisms that are detrimental to humans or other animals include, for example, species of Salmonella, Listeria, Clostridium, and Campylobacter.

In some embodiments, a plant-associated microorganism in a plant colonization efficiency screen is epiphytic, living on the surface of a plant or plant part, such as the leaves, roots, flowers, buds, seeds or fruits. In some embodiments, a plant-associated microorganism is an endophyte, living within a plant host for at least a portion of the microorganism's life cycle. In some embodiments, a plant-associated microorganism resides inside a plant host cell for at least a portion of the microorganism's life cycle.

In some embodiments, a host plant in a plant colonization efficiency screen is a crop plant. Crop host plants include, but are not limited to, corn, soybean, wheat, Brassica sp. (e.g., B. napus, B. rapa, B. juncea) including Canola varieties, alfalfa, rice, rye, sorghum, millet (e.g., pearl millet (Pennisetum glaucum)), proso millet (Panicum miliaceum), foxtail millet (Setaria italica), finger millet (Eleusine coracana), sunflower, safflower, tobacco, potato, peanuts, lentils, cotton, sweet potato (Ipomoea batatus), cassava, coffee, coconut, pineapple, citrus trees, cocoa, tea, banana, avocado, fig, guava, mango, olive, papaya, cashew, macadamia, almond, sugar beets, sugarcane, oats, barley, tomatoes, lettuce, green beans, lima beans, peas, cucurbits such as cucumber, cantaloupe, and musk melon, ornamentals, and conifers. Ornamental plant hosts that can be used in a plant colonization efficiency screen include, but are not limited to azalea, hydrangea, hibiscus, roses, tulips, daffodils, petunias, carnation, poinsettia, and chrysanthemum. Conifer host plants that can be used in a plant colonization efficiency screen include, but are not limited to, pines such as loblolly pine, slash pine, ponderosa pine, lodge pole pine, and Monterey pine; Douglas-fir; Western hemlock; Sitka spruce; redwood; true firs such as silver fir and balsam fir; and cedars such as Western red cedar and Alaska yellow-cedar. Turfgrass plant hosts include, but are not limited to, annual bluegrass, annual ryegrass, Canada bluegrass, fescue, bentgrass, wheatgrass, Kentucky bluegrass, orchard grass, ryegrass, redtop, Bermuda grass, St. Augustine grass, and zoysia grass. In some embodiments, the host plant in a plant colonization efficiency screen is a cereal plant selected from the group consisting of a rice, wheat, corn, barley, millet, sorghum, oat, and rye plant or plant part. Host plant parts that can be colonized include, but are not limited to, leaves, stems, flowers, roots, seeds, fruit, tubers, coleoptiles, and the like.

In some embodiments, an appropriate dose of the plant-associated microorganism for use in a colonization efficiency screen is determined in a dose-response evaluation using one or more strains to compare results using different dose applications. In some embodiments, an initial dose for evaluation is known from prior use of a strain or a related strain, for example as an agricultural inoculant. In some embodiments the plant-associated microorganism employed in a colonization screen is provided at a lower dose than typically used in agriculture in order to identify strains that would provide an advantage for commercial production. Production costs for agricultural inoculants can be reduced considerably when substantial colonization is achieved using a lower amount of microbial inoculant. In some embodiments, a dose of 10² to 10⁸ CFU of a microbial strain to be tested for colonization efficiency is applied to a plant or plant part. In some embodiments, a dose of 10³, 10⁴, 10⁵, 10⁶, or 10⁷ CFU is applied to a plant or plant part. In some embodiments, the dose is applied to a plant root, leaf, stem, seed, fruit or flower.

In some embodiments, a population of plant-associated microorganisms in a colonization screen will comprise a single strain to be assayed and a control strain. In some embodiments, two or more strains will be screened in a colonization assay with or without a control treatment to determine relative colonization efficiency of the strains in the screen. In some embodiments, a population of plant-associated microorganisms in a colonization screen will comprise two or more strains to be tested and a control, where the control can be a treatment where no microorganism is added to a sample, a treatment where a microorganism known or previously determined to be a poor colonizer of the target plant host is used, or a treatment where a microorganism known or previously determined to be an efficient colonizer of the target plant host is used. In some embodiments, both a control lacking added microbial strain and a control microbial strain known to be a poor colonizer may be used. In some embodiments, larger populations of strains are used in a colonization efficiency screen where results of the screen will be used to identify genetic elements associated with colonization efficiency as described herein.

In some embodiments, a population of plant-associated microorganisms used in a colonization efficiency screen is a population of strains from the same genus and species. In some embodiments, the population contains strains from the same genus, but includes strains of different species within the genus. In some embodiments, the population contains closely related strains from species that have been classified as belonging to different genera. By way of example, it has recently been proposed that some species of bacteria in the genus Methylobacterium should be reclassified as belonging to a new genus with the proposed name Methylorubrum. The species proposed to belong to the new genus are still highly related to other Methylobacterium species and a population of microorganisms for use in a colonization efficiency screen as described herein can contain species assigned to Methylobacterium or Methylorubrum or a mixed population with species assigned to one or the other of these general. Similar examples of plant-associated microorganisms that have been assigned to different genera but could be included in a population for screening for colonization efficiency by the methods described herein due their genetic similarity include, for example species of rhizobia, which may be Rhizobium, Sinorhizobium or Mesorhizobium. Populations of microorganisms for use in the present methods thus can include strains from the same or closely related genera in a single population to be screened for colonization efficiency.

The population in a plant colonization screen as described herein may be varied depending on factors including, for example, if the results of the screen will be used in association analyses, and the genetic relatedness of strains within the population. In some embodiments, a single strain is a plant-associated microorganism is assayed to determine colonization efficiency. In some embodiments, the colonization efficiency of a strain is determined by comparison to a control sample lacking an added microbial strain. In some embodiments, the colonization efficiency of a strain is determined by comparison to a control sample containing a strain previously identified as either a poor colonizer or an efficient colonizer. In some embodiments, a population will include more than one strain to be screened for colonization efficiency. In some embodiments, a population will include 2, 3, 4, 5, 6, 7, 8, 9, 10, 15, 20 or more strains to be screened for colonization efficiency. In some embodiments, the population will include native strains obtained from a plant environment. In some embodiments, the population will include isolates of a strain that has been mutagenized by either directed or random mutagenesis. In some embodiments, a population will include mutagenized isolates of a strain identified as an efficient colonizer and isolates of the strain having even greater colonization efficiency can be identified. In some embodiments, a strain identified as beneficial to a plant host, but lower in colonization efficiency than other genetically related strains is mutagenized to generate a population that can be screened to identify an isolate of the strain that has improved colonization efficiency.

In some embodiments, results of the colonization assay are used in association analyses and strains in the population are genetically related, such as members of the same microbial species. In some embodiments, results of the colonization assay are used in association analyses and strains in the population are genetically related, such as members of the same microbial species. In some embodiments, strains in the population are genetically related and results of the colonization assay are used in association analyses to identify genetic elements associated with colonization efficiency. In some embodiments, all strains in the population are members of the same microbial species, as identified for example by comparing the relatedness of genome sequences of the strains. In some embodiments, the strains in the population will have average nucleotide identity (ANI) values of about 95%. In some embodiments, the strains in the population will have ANI values of about 96%. In some embodiments, the strains in the population will have ANI values of about 97-99%. In some embodiments, the strains in the population will have ANI values of about 98-99%. In some embodiments, the strains in the population will have ANI values of about 99.1, 99.2, 99.3, 99.4, 99.5, 99.6, 99.7, 99.8, or 99.9%

In some embodiments, the strains will be members of different species of the same genus, or different species of two or more closely related genera. In some embodiments, strains of different species that find use in the methods herein will have ANI values equal to or greater than 80%, 85%, 90%, and 95%.

In some embodiments, the population size is sufficient to allow for identification of at least 5 strains that efficiently colonize the plant host or plant host part and at least 5 strains that are poor colonizers of the plant host or plant host part for use in association analyses. In embodiments where the population comprises more distantly related microbial strains, the population will be of a sufficient size to allow for identification of 10 or more strains that efficiently colonize the plant host or plant host part and 10 or more strains that are poor colonizers of the plant host or plant host part. In some embodiments, the population in a plant colonization efficiency screen will comprise 10 or more distinct strains. In some embodiments, the population in a plant colonization efficiency screen will comprise 20 or more distinct strains. In some embodiments, the population in a plant colonization efficiency screen will comprise 50 or more distinct strains. In some embodiments, the population in a plant colonization efficiency screen will comprise 100 or more distinct strains. In some embodiments, the population in a plant colonization efficiency screen will comprise 200 or more distinct strains. In some embodiments, the population in a plant colonization efficiency screen will comprise 300, 400, or 500 or more distinct strains. In some embodiments, each of the strains in the population is screened in a separate experiment. In some embodiments, multiple strains that can be distinguished, for example by the presence of genetic markers or differences in appearance of colonies may be screened in a single experiment.

In some embodiments, the population of plant-associated microorganisms comprises 100 or more strains of Methylobacterium and results of the screen are used in association analyses to identify genetic elements associated with colonization efficiency. In some embodiments, the plant is a soybean plant. In some embodiments, the plant part treated in a colonization efficiency screen is a soybean seed. In some embodiments, the dose used to treat a plant or plant part in a colonization screen is 10⁵ or 10⁶ CFU. In some embodiments, the plant part sampled to determine colonization efficiency is a plant shoot. In some embodiments, the plant is corn. In some embodiments, the plant part treated in a colonization efficiency screen is a corn seed. In some embodiments, the dose used in a colonization screen is 10⁵ or 10⁶ CFU. In some embodiments, the plant part sampled to determine colonization efficiency is a plant root.

In some embodiments, a population of plant-associated microorganisms comprises two or more strains of Methylobacterium and results of colonization efficiency screens are used to identify microbial strains for testing in field studies for use of the strains as agricultural inoculants. In some embodiments, additional screens are employed to further evaluate the fitness of plant-associated microorganisms that efficiently colonize a host plant for use as an agricultural inoculant on the host crop plant and additional crop plants. In some embodiments, the plant is a corn plant. In some embodiments, the plant part treated in a colonization efficiency screen is a corn seed. In some embodiments, the dose used to treat a plant or plant part in a colonization screen is 10⁵ or 10⁶ CFU. In some embodiments, the plant part sampled to determine colonization efficiency is a plant root. In some embodiments, the plant is a soybean plant. In some embodiments, the plant part treated in a colonization efficiency screen is a soybean seed. In some embodiments, the dose used to treat a plant or plant part in a colonization screen is 10⁵ or 10⁶ CFU. In some embodiments, the plant part sampled to determine colonization efficiency is a plant shoot.

The performance of each strain in a population screened for colonization efficiency is evaluated and quantified to provide a dataset that can be used to identify strains having significantly elevated colonization efficiency as compared to a control, and to identify the strains which demonstrate the lowest ability to colonize the plant host.

In some embodiments, efficiency of colonization is evaluated by inoculating a host plant, plant part or plant tissue with a known quantity of the plant-associated microorganism and assessing the titer of the microorganism on the plant after a period of growth. In other embodiments, a known quantity of the plant-associated microorganism to be tested for colonization efficiency is added to soil in which a host plant is grown, or provided in water or nutrients that are supplied to the host plant. In some embodiments, a plant seed is inoculated, and colonization efficiency is assessed on plant tissue above the soil level, on plant tissue below the soil level, or in plant seed tissue, for example where the plant-associated microorganism is an endophyte. Titers or genome copies of the plant-associated microorganism are determined and compared to control samples to determine colonization density. In some embodiments, a target host plant is grown in soil that has been treated, for example sterilized, to remove other microorganisms from the sample. In some embodiments, a target plant host is grown in soil which contains a naturally occurring soil microflora. In some embodiments, a target plant host is grown in non-soil media that include calcined clay that has limited background microbiota. In some embodiments, a control sample containing only naturally occurring soil microflora may demonstrate higher colonization densities than some or all of the strains in the population being tested. In some embodiments, the inclusion of a second control treatment is used to ensure that a useful control is available for each treatment. In some embodiments, a second control treatment contains a plant-associated microorganism previously determined to be a poor or inefficient colonizer of the target host plant. In some embodiments, a plant associated-microorganism used in a second control sample is from the same genus or species as the population of plant-associated microorganisms in the screen.

In some embodiments, colonization efficiency is assessed after the plant has been allowed to grow (e.g., in a controlled environment), for about one to two weeks to allow for germination and seedling growth. In some embodiments, a plant seed is inoculated, and colonization efficiency is assessed by determining the titer of the plant-associated microorganism on plant tissue below the soil level and comparing to a control. In some embodiments, root colonization efficiency is assessed after the plant has been allowed to grow (e.g., in a controlled environment) for at least a week, or alternatively for two to three weeks. In some embodiments, selective media can be used to determine the population of the plant-associated microorganism that has colonized the test plant, plant part or plant tissue. In one embodiment, the plant-associated microorganism is a Methylobacterium, and colonizing bacteria can be recovered from the plant sample and plated onto AMS-MC media. After incubation for a sufficient time to allow the Methylobacterium to grow, pink colonies indicative of Methylobacterium can be counted to determine the number of Methylobacterium colonies. In some embodiments, results or scores, for each strain in the population are recorded as CFU per unit of plant tissue, for example CFU per mg of fresh weight.

In some embodiments, quantification to allow comparison of the colonization efficiency of the population of strains is accomplished using methods other than direct plating to titer the plant-associated microorganisms. In some embodiments, genome sequencing is used to determine the relative numbers of plant-associated microorganism that have colonized the test plant, plant part or plant tissue. In some embodiments, 16S rRNA gene sequencing for bacteria or 18S rRNA gene sequencing for fungi, is implemented to quantitate and compare colonization efficiency of different strains in the population being screened. In some embodiments, qPCR is used to quantitate the plant-associated microorganisms that have colonized the host plant, plant part or plant tissue using markers that are known or have been specifically developed for the strains in the population being screened. In some embodiments, results are recorded for each strain in the population as copy number where quantification involves sequence analysis or cycle threshold (ct) values where qPCR is employed.

In some embodiments, strains of plant-associated microorganisms can be labeled with a marker in order to simplify detection and quantification of the microorganism on the host plant or plant part. Useful markers include both selectable and screenable markers. Selectable markers are generally genes which encode proteins that confer resistance to antibiotics, thus allowing detection of strains containing the marker by the ability to grow in media containing antibiotics. Selectable markers useful in the methods described herein include those conferring resistance to kanamycin, gentamycin, ampicillin, and chloramphenicol as well as other antibiotics known to be active against gram negative bacteria. Screenable markers, also referred to as reporter genes, encode proteins that cause a change in visible characteristics of a bacterial colony, for example, a change in color. Examples of screenable markers that find use in the methods described herein are lacZ, GUS, GFP, mcherry, and the like. Markers can be incorporated into the genome of the strains in the population to be screened, or can be provided on a plasmid that is inserted into the plant-associated microorganisms. Insertion of a plasmid can be accomplished for example, by conjugation, electroporation or other transformation methods known in the art. In some embodiments, where markers are employed results are recorded for each strain in the population based on colorimetric, fluorescent, or luminescent activity depending on the marker employed.

Following quantification of each strain in a population in a colonization efficiency screen, a first set of strains is selected that colonize the plant part or host at enhanced densities as compared to a control. In some embodiments, the strains in the first set will meet a threshold of statistical significance, for example having a p-value of p<0.10, or having a p-value of p<0.05. In some embodiments, a first set of strains will be determined as those having colonization efficiency scores in the highest 20% of the population, the highest 10% of the population, or the highest 5% of the population.

Additionally, a second set of strains is identified, where the strains in the second set are inefficient colonizers of the plant host or plant host part used in the colonization efficiency screen. Strains in the second set display quantitatively lower CFU/mg than a control in the colonization efficiency screen. In some embodiments, strains in the second set representing members of the population with the lowest colonization density scores are present at a density at least one log lower than that of the efficient colonizers in the first set of strains. In some embodiments, a second set of strains will be determined as those having colonization efficiency scores in the lowest 20% of the population, the lowest 10% of the population, or the lowest 5% of the population.

In certain embodiments, genetic elements correlated with colonization efficiency are identified by comparing the sequences of genetic elements in said first set of strains and said second set of strains to identify genetic elements that are correlated with the trait of colonization efficiency. In some embodiments, a genome-wide association study, or whole genome association study is performed to identify the correlated genetic elements.

In some embodiments where whole genome association is used to identify genetic elements that correlate with colonization efficiency, a pan-genome is generated for the population being tested, for example as described by Page et al. (Bioinformatics (2015) 31:3691-3693). In some embodiments, a pan-genome is generated using a higher number of plant-associated microorganisms than is present in the population being tested. In some embodiments, a pan-genome is generated using a population of 50 or more different strains of related plant-associated microorganisms. In some embodiments, a pan-genome is generated using 100 or more different strains of related plant-associated microorganisms. In some embodiments, a pan-genome is generated using sequences of 200 or more different strains of related plant-associated microorganisms. In some embodiments, a pan-genome is generated using sequences of 300, 400, 500 or more, or even greater than 1000 different strains of related plant-associated microorganisms.

Where the population of plant-associated microorganisms contains strains from multiple different related species or genera, the conditions for generation of the pan genome are modified to account for the lower homology among the genetic elements and/or encoded proteins of the strains in the population. In one embodiment, the population of plant-associated microorganisms contains strains from a single species, and the sequence identity cutoff in a BLASTP analysis is 90, 91, 92, 93, 94, 95, 96, 97, 98, or 99% identity. In some embodiments, the population contains strains from multiple different, but related species, and the sequence identity cutoff is reduced depending on the phylogenetic relatedness of the strains in the population. For example, a cutoff of sequence identity of 90%, 85%, 80%, 75%, 70%, 65%, 60%, 55%, or 50% can be used.

In some embodiments, using the pan-genome as a reference, the presence or absence of each genetic element in the first set of strains (efficient colonizers) and the second set of strains (poor or undetectable colonizers) is determined. The presence and absence scores are used in a correlation analysis to identify the genetic elements that correlate positively with colonization efficiency. In some embodiments, multiple different p-values are generated and evaluated in the analysis, for example raw p-value, Bonferroni p-value, Benjamini-Hochberg p-value, and empirical p-value. In some embodiments, correlation is established using a statistical significance threshold based on empirical p-value where a cutoff of p less than or equal to 0.05 or p less than or equal to 0.10 is used. In some embodiments, scores for sensitivity, where the presence of the gene is used as a determination that a strain is an efficient colonizer, and/or specificity, where the non-presence or absence of the gene is used as an indicator that a strain is a poor colonizer, are also used in the correlation analysis. In some embodiments, a cutoff of greater than or equal to 10, 20 or 25% sensitivity is used. In some embodiments, a cutoff of greater than or equal to 50, 60, 70, or 75% specificity is used. In other embodiments, higher or lower sensitivity and specificity cutoffs are employed.

In some embodiments, genetic elements identified as correlated with colonization efficiency will have a predicted function based on homology to previously identified gene or protein sequences. In some embodiments, genetic elements identified as correlated with colonization efficiency will encode a protein annotated as a “hypothetical protein”, either from literature-based homologs annotated as such, or based on lack of homology to any publicly known sequence.

In certain embodiments, genetic elements positively correlated with plant colonization efficiency are used to identify additional plant-associated microorganisms that contain the genetic elements, for example as a prescreen for selection of strains to be tested for use as an agricultural inoculant. In this manner, strains are identified that, based on the presence of the correlated genes, are capable of growth and reproduction on a target host plant or host plant part. In some embodiments, the additional plant-associated microorganisms will be identified by the presence of two or more genetic elements that are positively correlated with colonization efficiency.

Detection of genetic elements positively correlated with plant colonization efficiency can be accomplished by a nucleic acid detection technique. In certain embodiments, the nucleic acid detection technique detects a sequence encoding a protein of SEQ ID NO:1 to SEQ ID NO: 45, a sequence encoding a protein having at least 50%, 60%, 70%, 80%, 85%, 90%, 95%, 97%, 98%, 99%, or 100% sequence identity to SEQ ID NO: 91 to SEQ ID NO: 135, or a nucleic acid sequence having at least 50%, 60%, 70%, 80%, 85%, 90%, 95%, 97%, 98%, 99%, or 100% sequence identity to SEQ ID NO: 46 to SEQ ID NO: 90 or a fragment thereof. Applicable nucleic acid detection techniques include nucleic acid hybridization with one or more of SEQ ID NO: 46 to SEQ ID NO: 90 provided herein of fragments thereof (e.g., fragments comprising at least 15, 18, 20, 50, or 100 to about 500 or more nucleotides of SEQ ID NO: 46 to SEQ ID NO: 90). Appropriate stringency conditions which promote DNA hybridization are, for example, 6× sodium chloride/sodium citrate (SSC) at about 45° C., followed by a wash of 2×SSC at 50° C. Such conditions are known to those skilled in the art and can be found, for example in Current Protocols in Molecular Biology, John Wiley & Sons, N.Y. (1989). Salt concentration and temperature in the wash step can be adjusted to alter hybridization stringency. For example, conditions may vary from low stringency of about 2×SSC SSC at 40° C. to moderately stringent conditions of about 2×SSC at 50° C. to high stringency conditions of about 0.2×SSC at 50° C. Other nucleic acid detection techniques that can be used to detect genetic elements positively correlated with plant colonization efficiency include PCR amplification using primers designed from protein and/or nucleic acid sequences provided herein, or direct genome sequencing. Other nucleic acid detection techniques that find use in detection of sequences in a target microorganism include polymerase chain reaction, branched DNA, ligase chain reaction, transcription mediated amplification (TMA), nucleic acid sequence-based amplification (NASBA), nanopore-, mass spectroscopy, or direct sequencing based methods, or any combination thereof. Analysis of genome sequences to detect genetic elements positively correlated with plant colonization efficiency can be by comparison of nucleic acid sequences to sequences of genetic elements positively correlated with plant colonization efficiency provided herein, or by analysis of proteins encoded by nucleic acid sequences present in the genomes of plant-associated microorganisms. Sequences of genetic elements or proteins identified in this manner can be compared using standard nucleic acid protein sequence and analysis tools, including for example, BLAST, pFAM, ClustalW, ALLALIGN, DNASTAR, SIM, SEQALN, NEEDLE, SSEARCH, and the like.

In some embodiments, presence of a genetic element associated with plant colonization is detected where a genetic element in a plant-associated microorganism of interest encodes a protein having at least 50%, 60%, 70%, 75%, 80%, 85%, 90%, 95% sequence identity or more to a protein encoded by a genetic element correlated with colonization efficiency. In certain embodiments, the genetic element comprises a gene that encodes a protein having at least 50%, 60%, 70%, 75%, 80%, 85%, 90%, 95% sequence identity or more to a protein encoded by SEQ ID NO: 1 to SEQ ID NO: 45. In some cases where the plant-associated microorganism of interest is more distantly related to strains in the population screened for colonization efficiency, identity to a genetic element correlated with colonization efficiency may be less than 50%, for example, 40% or even 30%. In certain embodiments, the genetic element comprises a gene that encodes a protein having 30% to 50% sequence identity to a protein encoded by SEQ ID NO: 1 to SEQ ID NO: 45. In certain embodiments, a genetic element correlated with colonization efficiency can encode a protein with the biochemical activity (e.g., transcription regulatory, enzymatic, transport, receptor, and/or binding activity) of a gene as set forth and annotated in Table 2. In certain embodiments, a genetic element correlated with colonization efficiency can encode a protein with the biochemical activity (e.g., transcription regulatory, enzymatic, transport, receptor, and/or binding activity) of a gene encoding a protein of SEQ ID NO: 1 to SEQ ID NO: 45. In certain embodiments, a genetic element correlated with colonization efficiency can encode a protein with any of the aforementioned sequence identities to SEQ ID NO: 1 to SEQ ID NO: 45 and the biochemical activity (e.g., transcription regulatory, enzymatic, transport, receptor, and/or binding activity) of a gene encoding a protein of SEQ ID NO: 1 to SEQ ID NO: 45.

In some embodiments, presence of a genetic element associated with plant colonization is detected where a genetic element in a plant-associated microorganism of interest comprises a nucleic acid sequence having at least 50%, 60%, 70%, 75%, 80%, 85%, 90%, 95%, 97%, 98%, 99%, or 100% sequence identity to any one of SEQ ID NO: 46 to SEQ ID NO:90. In some cases where the plant-associated microorganism of interest is more distantly related to strains in the population screened for colonization efficiency or to the Methylobacterium strains which contain any one of SEQ ID NO: 46 to SEQ ID NO:90, sequence identity to a genetic element correlated with colonization efficiency may be less than 50%, for example, 40% or even 30%. In certain embodiments, the genetic element comprises a gene that comprises a nucleic acid sequence having 30% to 50% sequence identity to any one of SEQ ID NO: 46 to SEQ ID NO:90.

In some embodiments, presence of a genetic element associated with plant colonization is detected where a genetic element in a plant-associated microorganism of interest encodes a protein having at least 50%, 60%, 70%, 75%, 80%, 85%, 90%, 95%, 97%, 98%, 99%, or 100% sequence identity to any one of SEQ ID NO: 91 to SEQ ID NO: 135. In some cases where the plant-associated microorganism of interest is more distantly related to strains in the population screened for colonization efficiency or to the Methylobacterium strains which contain genetic elements that encode any one of SEQ ID NO: 91 to SEQ ID NO: 135, sequence identity to a protein encoded by a genetic element correlated with colonization efficiency may less than 50%, for example, 40% or even 30% to any one of SEQ ID NO: 91 to SEQ ID NO: 135. In certain embodiments, the genetic element comprises a gene that encodes a protein having 30% to 50% sequence identity to a protein encoded by any one of SEQ ID NO: 46 to SEQ ID NO: 90 (i.e., the protein of SEQ ID NO: 91 to SEQ ID NO: 135, respectively). In certain embodiments, a genetic element correlated with colonization efficiency can encode a protein with any of the aforementioned sequence identities to SEQ ID NO: 91 to SEQ ID NO: 135 and the biochemical activity (e.g., transcription regulatory, enzymatic, transport, receptor, and/or binding activity) of a protein of SEQ ID NO: 91 to SEQ ID NO: 135. In some embodiments, presence of a genetic element associated with plant colonization is detected where a genetic element in a plant-associated microorganism is identified, for example by sequence comparison to known DNA or protein sequence database, as encoding a protein selected from the proteins listed in Table 1 below. Useful methods to compare sequences include, for example, TBLASTN, where the protein sequences are aligned to a nucleotide database in all six reading frames, and BLASTP, where a protein query is used to search a protein database directly.

TABLE 1

| Gene | |
|---|---|
| name | Annotation |
| cdhR | HTH-type transcriptional regulator CdhR |
| amaB | N-carbamoyl-L-amino acid hydrolase |
| rbn | Ribonuclease BN |
| bbsG | (R)-benzylsuccinyl-CoA dehydrogenase |
| fecA | Vitamin B12 transporter BtuB |
| luxQ | Autoinducer 2 sensor kinase/phosphatase LuxQ |
| mdlC | Benzoylformate decarboxylase |
| hddA | D-glycero-alpha-D-manno-heptose 7-phosphate kinase |
| fptA | Fe(3+)-pyochelin receptor |
| livF | High-affinity branched-chain amino acid transport ATP- |
| | binding protein LivF |
| hddC | D-glycero-alpha-D-manno-heptose 1-phosphate |
| | guanylyltransferase |
| sutR | HTH-type transcriptional regulator SutR |
| ftsY | Signal recognition particle receptor FtsY |
| bicA | C4-dicarboxylic acid transporter DauA |
| hdhA | 7-alpha-hydroxysteroid dehydrogenase |
| ssuA | Putative aliphatic sulfonates-binding protein |
| uvrA | UvrABC system protein A |
| gpmA_2 | 2,3-bisphosphoglycerate-dependent phosphoglycerate mutase |
| ecfG_1 | ECF RNA polymerase sigma factor EcfG |
| adh | putative zinc-binding alcohol dehydrogenase |
| lgt | Prolipoprotein diacylglyceryl transferase |
| yfih | Laccase domain protein |
| cyaA | Adenylate cyclase 1 |
| vgb_3 | Virginiamycin B lyase |
| pimB_2 | GDP-mannose-dependent alpha-(1-6)-phosphatidylinositol |
| | monomannoside mannosyltransferase |
| bmr3_2 | Multidrug resistance protein 3 |
| fabD_1 | Malonyl CoA-acyl carrier protein transacylase |

In some embodiments, protein sequences identified herein are used to identify genetic elements associated with plant colonization in a microbial strain of interest. Provided herein as SEQ ID NO: 1 to SEQ ID NO: 45 are consensus sequences representing Methylobacterium proteins encoded by genetic elements associated with colonization of soybean phyllosphere or corn rhizosphere. In some embodiments, SEQ ID NO: 1 to SEQ ID NO: 45 are used to identify additional Methylobacterium strains having genetic elements associated with plant colonization efficiency.

In some embodiments, additional plant-associated microorganisms will be identified by the presence of 1, 2, 3, 4, 5, 6, 7, 8, 9, or 10 or more genetic elements that are positively correlated with colonization efficiency. In some embodiments, a plant-associated microorganism for use on a particular crop host plant or plant part will be selected based on the presence of the genetic elements that are most closely correlated with colonization efficiency of the target plant host. In some embodiments additional plant-associated microorganisms will be selected for use in agricultural testing that have the highest number of correlated genes identified.

In some embodiments additional plant-associated microorganisms that are selected based on the presence of genetic elements that correlate with colonization efficiency will be from a species that was represented in a population used in the colonization efficiency screen. In some embodiments, additional plant-associated microorganisms that are selected are from more distantly related species. In some embodiments where the microorganisms are more distantly related, the presence of a higher number of genetic elements associated with colonization efficiency may be required to identify additional plant-associated microorganisms capable of efficient plant colonization.

In some embodiments, a plant-associated microorganism having enhanced colonization efficiency will be obtained by genetically transforming a target strain to contain genetic elements that correlate with colonization efficiency. In some embodiments, transformation of Methylobacterium strains can be accomplished by electroporation or conjugation to transfer of vectors, gene-containing fragments, or expression constructs from one Methylobacterium strain to a second bacterial strain (e.g., a second Methylobacterium strain) as described, for example in co-pending application 62/760,092. In some embodiments, a plant-associated microorganism having enhanced colonization efficiency is genetically transformed to include genetic elements that can confer pest tolerance (e.g., insect, nematode, and/or fungal pathogen tolerance) or herbicide tolerance. In some embodiments, transformation of an enhanced colonization efficiency Methylobacterium strain to include genetic elements that can confer pest tolerance (e.g., insect, nematode, and/or fungal pathogen tolerance) or herbicide tolerance as described, for example in co-pending application 62/694,775.

In some embodiments where the plant-associated microorganism identified as capable of efficiently colonizing a plant is beneficial to a plant host, additional screens are employed to further evaluate the fitness of the plant-associated microorganism for use as an agricultural inoculant. In some embodiments, such further evaluations are conducted prior to or subsequent to screening a population of plant-associated microorganisms for colonization efficiency.

In some embodiments a screen for desiccation tolerance is employed. Methods for identifying desiccation tolerant microorganisms include screening a population of microorganisms for viability after a period of drying, for example, in one embodiment drying under a laminar flow hood, and comparing viability to other tested strains. In some embodiments, plant-associated microorganisms can be dried directly from the growth medium, for example, in one embodiment, dried in petri dishes or microtiter plates. In some embodiments, the microorganisms are grown in media having a single carbon source, dried in the minimal media and rehydrated in a rich nutrient media. In some embodiments, plant-associated microorganisms are coated on seeds and allowed to dry and tested for viability after a period of storage on dry seeds. In some embodiments, microorganisms are stored on dry seeds for anywhere from one day to three weeks, including 2 days, 5 days, 1 week, 2 weeks, and 3 weeks or more before testing for viability. In some embodiments, microorganisms are stored on dry seeds for greater than 4 weeks prior t testing for viability. In some embodiments, plant-associated microorganisms are tested for production of exopolysaccharide (EPS) which has been shown to be involved in protection from desiccation (Gasser et al. 2009, FEMS Microbiol Ecol 70:142-150).

In some embodiments, a screen for the ability of a plant-associated microorganism to tolerate the presence of commonly used agricultural chemicals is used. In some embodiments, microorganisms to be tested for tolerance to agricultural chemicals will be grown in liquid media and spotted onto solid media plates containing the agricultural chemicals. In some embodiments, the agricultural chemicals in such a screen will include herbicides, for example one or more of the following acetochlor, clethodim, dicamba, flumioxazin, fomesafen, glyphosate, glufosinate, mesotrione, quizalofop, saflufenacil, sulcotrione, and 2,4-D. In some embodiments, the agricultural chemicals in such a screen will include fungicides, for example one or more of the following acibenzolar-S-methyl, azoxystrobin, benalaxyl, bixafen, boscalid, carbendazim, cyproconazole, dimethomorph, epoxiconazole, fluopyram, fluoxastrobin, flutianil, flutolanil, fluxapyroxad, fosetyl-Al, ipconazole, isopyrazam, kresoxim-methyl, mefenoxam, metalaxyl, metconazole, myclobutanil, orysastrobin, penflufen, penthiopyrad, picoxystrobin, propiconazole, prothioconazole, pyraclostrobin, sedaxane, silthiofam, tebuconazole, thifluzamide, thiophanate, tolclofos-methyl, trifloxystrobin, and triticonazole. In some embodiments, the agricultural chemicals in such a screen will include insecticides and/or nematicides, including, for example abamectin, aldicarb, aldoxycarb, bifenthrin, carbofuran, chlorantraniliporle, chlothianidin, cyfluthrin, cyhalothrin, cypermethrin, deltamethrin, dinotefuran, emamectin, ethiprole, fenamiphos, fipronil, flubendiamide, fosthiazate, imidacloprid, ivermectin, lambda-cyhalothrin, milbemectin, nitenpyram, oxamyl, permethrin, tioxazafen, spinetoram, spinosad, spirodichlofen, spirotetramat, tefluthrin, thiacloprid, thiamethoxam, tioxazafen, and thiodicarb. In some embodiments, the agricultural chemicals in such a screen will include biocides, such as isothiazolinones, including for example 1,2 Benzothiazolin-3-one (BIT), 5-Chloro-2-methyl-4-isothiazolin-3-one (CIT), 2-Methyl-4-isothiazolin-3-one (MIT), octylisothiazolinone (OIT), dichlorooctylisothiazolinone (DCOIT), and butylbenzisothiazolinone (BBIT); 2-Bromo-2-nitro-propane-1,3-diol (Bronopol), 5-bromo-5-nitro-1,3-dioxane (Bronidox), Tris(hydroxymethyl)nitromethane, 2,2-Dibromo-3-nitrilopropionamide (DBNPA), and alkyl dimethyl benzyl ammonium chlorides. In some embodiments, the agricultural chemicals in such a screen will include any combination of fungicides, herbicides, insecticides nematicides, and biocides.

In some embodiments, a screen for the ability of a plant-associated microorganism to grow robustly and to a high titer in media comprising varying sources, concentrations and combinations of carbon, nitrogen and other nutrients, including one or more of the following vitamins or other trace elements, is employed. Such screens find particular interest, for example, where a desirable plant-associated microorganism is known to have a relatively slow growth rate.

Microbial strains identified as capable of efficiently colonizing a plant host in a screen as defined herein and/or identified as containing genes associated with plant colonization using the methods provided herein, are tested under agricultural field conditions to identify microbial strains that confer increased yield to inoculated host plants. In some embodiments, microbial strains capable of efficiently colonizing a plant host and/or containing genes associated with enhanced colonization efficiency are applied using a lower inoculum dose than typically used in agricultural applications for related strains not selected as having enhanced colonization efficiency. In some embodiments, a dose for a microbial strain capable of efficiently colonizing a plant host will be 95% lower than a dose used for a related inoculant. In some embodiments, a dose for a microbial strain capable of efficiently colonizing a plant host will be 90% lower than a dose used for a related inoculant. In some embodiments, a dose for a microbial strain capable of efficiently colonizing a plant host will be 85%, 80% or 75% lower than a dose used for a related inoculant. In some embodiments, a dose for a microbial strain capable of efficiently colonizing a plant host will be anywhere from about 25% to 75% lower than a dose used for a related inoculant.

In some embodiments, agricultural field tests are conducted in a microplot to evaluate yield enhancement of a large number of strains. In some embodiments, the size of a microplot is two 30-inch rows, each 10 feet in length, although many variations are possible. In some embodiments, a microplot trial will be conducted at 2 or more sites with four or more replications at each site. In some embodiments, a microplot trial will be conducted at 4 or more sites with four, five or six or more replications at each site. In some embodiments, the sites selected for the microplot trial will be geographically diverse to allow for yield analysis under different environmental conditions. In some embodiments, 40 or more strains are evaluated in a microplot to identify yield-enhancing strains. In some embodiments, 50, 60, 70, 80, 90, 100 or more strains are tested for yield enhancing properties in a microplot trial.

In some embodiments, large-scale conventional field trials are conducted to evaluate and/or confirm yield-enhancing capabilities of strains having enhanced colonization efficiency and/or identified as containing genes associated with plant colonization. In some embodiments, the size of a conventional field trial is four 30-inch rows, each 20 feet or 40 feet in length, although many variations are possible. In some embodiments, a large-scale field trial will be conducted at four or more sites with six or more replications at each site. In some embodiments, the sites selected for the conventional field trials will be geographically diverse to allow for yield analysis under different environmental conditions. In some embodiments, fewer strains are evaluated in a large-scale field trial as compared to the number of strains that are evaluated in a microplot. In some embodiments, 2 or more, 5 or more, 10 or more strains, 15 or more, or 20 or more strains are evaluated in a large-scale field trial.

In some embodiments, candidate yield-enhancing microbial strains will have additional traits that make them amenable to use in agriculture, including for example desiccation tolerance, tolerance to one or more agricultural chemicals, and robust growth. In some embodiments, the yield-enhancing candidate microbial strains are identified as a hit in one or more screens for desiccation tolerance, agricultural chemical tolerance or robust growth as described herein. In some embodiments, the yield-enhancing candidate microbial strains are identified as hits in desiccation tolerance and agricultural chemical tolerance screens. In some embodiments, the yield-enhancing candidate microbial strains are identified as tolerant to one or more fungicides, herbicides, insecticides nematicides, and biocides.

In some embodiments, the plant associated microbial strain is a Methylobacterium strain. In some embodiments, a Methylobacterium strain evaluated in plant field trials will demonstrate a high degree of fitness for use as agricultural inoculants as the result of enhanced colonization efficiency and tolerance to conditions and chemicals that are common to various plant inoculation practices, including for example seed treatment. The Methylobacterium strains identified as providing for increased yield in treated plants and having enhanced colonization efficiency when the seed or soil near the seed is treated with such strains, will have higher population densities on target plants or plant parts, reduced costs of application due to the ability to use less inoculum, or enhanced tolerance to agricultural environments.

Compositions useful for treatment of plants or soil in which plants are grown are also provided. In some embodiments, Methylobacterium strains identified as providing for increased yield in treated plants and having enhanced colonization efficiency will find use in agriculture as inoculants for treatments of plants or plant parts. In some embodiments, an effective amount of the strain having enhanced colonization efficiency used in treatment of seeds or plant parts is provided in a composition having a Methylobacterium titer of at least about 1×10⁶ colony-forming units per milliliter, at least about 5×10⁶ colony-forming units per milliliter, at least about 1×10⁷ colony-forming units per milliliter, at least about 5×10⁸ colony-forming units per milliliter, at least about 1×10⁹ colony-forming units per milliliter, at least about 1×10¹⁰ colony-forming units per milliliter, or at least about 3×10¹⁰ colony-forming units per milliliter. In some embodiments, an effective amount of the Methylobacterium strain having enhanced colonization efficiency used in treatment of seeds or plant parts is provided in a composition with the Methylobacterium at a titer of about least about 1×10⁶ colony-forming units per milliliter, at least about 5×10⁶ colony-forming units per milliliter, at least about 1×10⁷ colony-forming units per milliliter, or at least about 5×10⁸ colony-forming units per milliliter to at least about 6×10¹⁰ colony-forming units per milliliter of a liquid or an emulsion. In some embodiments, an effective amount of the Methylobacterium strain having enhanced colonization efficiency used in treatment of seeds or plant parts is provided in a composition with the Methylobacterium titer of at least about 1×10⁶ colony-forming units per gram, at least about 5×10⁶ colony-forming units per gram, at least about 1×10⁷ colony-forming units per gram, or at least about 5×10⁸ colony-forming units per gram to at least about 6×10¹⁰ colony-forming units of Methylobacterium per gram of the composition. In some embodiments, an effective amount of a Methylobacterium strain having enhanced colonization efficiency is provided in a composition with a Methylobacterium titer of at least about 1×10⁶ colony-forming units per gram, at least about 5×10⁶ colony-forming units per gram, at least about 1×10⁷ colony-forming units per gram, or at least about 5×10⁸ colony-forming units per gram to at least about 6×10¹⁰ colony-forming units of Methylobacterium per gram of particles in the composition containing the particles, wherein the particles comprise a solid substance wherein a mono-culture or co-culture of a Methylobacterium strain having enhanced colonization efficiency is adhered thereto. In some embodiments, an effective amount of a Methylobacterium strain provided herein can be a composition with a Methylobacterium titer of at least about 1×10⁶ colony-forming units per mL, at least about 5×10⁶ colony-forming units per mL, at least about 1×10⁷ colony-forming units per mL, or at least about 5×10⁸ colony-forming units per mL to at least about 6×10¹⁰ colony-forming units of Methylobacterium per mL in a composition comprising an emulsion wherein a mono-culture or co-culture of a Methylobacterium strain having enhanced colonization efficiency is adhered to a solid. In some embodiments, an effective amount of a Methylobacterium strain having enhanced colonization efficiency can be provided in a composition with a Methylobacterium titer of at least about 1×10⁶ colony-forming units per mL, at least about 5×10⁶ colony-forming units per mL, at least about 1×10⁷ colony-forming units per mL, or at least about 5×10⁸ colony-forming units per mL to at least about 6×10¹⁰ colony-forming units of Methylobacterium per mL of in a composition comprising an emulsion wherein a mono-culture or co-culture of a Methylobacterium strain having enhanced colonization efficiency is provided therein or grown therein.

An effective amount of a Methylobacterium strain having enhanced colonization efficiency provided in a treatment of a seed or plant part is an amount that results in an increase in the colonization density of the Methylobacterium strain on a plant grown from the treated seed or plant comprising the treated plant part, and a resulting increase in plant performance, for example as measured by plant yield. In some embodiments, an effective amount of a Methylobacterium strain having enhanced colonization efficiency provided in a treatment of a seed or plant part will be lower than the amount used for a similar inoculant that is not an efficient colonizer, and is at least about 10³, 10⁴, 10⁵, 10⁶, 10⁷, or 10⁸ CFU per seed or treated plant part. In some embodiments, the effective amount of Methylobacterium provided in a treatment of a seed or plant part is an amount where the CFU per seed or treated plant part will exceed the number of CFU of any resident naturally occurring Methylobacterium strain by at least 5-, 10-, 100-, or 1000-fold. In some embodiments, the effective amount of Methylobacterium provided in a treatment of a seed or plant part is an amount where the CFU per seed or treated plant part will exceed the number of CFU of any resident naturally occurring Methylobacterium by at least 2-, 3—, 5-, 8-, 10-, 20-, 50-, 100-, or 1000-fold.

### EXAMPLES

The following examples are included to demonstrate certain embodiments of the disclosure. However, those of skill in the art should, in light of the instant disclosure, appreciate that many changes can be made in the specific embodiments that are disclosed, while still obtaining like or similar results, without departing from the scope of the disclosure.

### Example 1 Dose Response Evaluation

To determine an appropriate target concentration of Methylobacterium for use in a colonization efficiency screen, seven strains previously identified as either having the ability to colonize soybean significantly at 10⁶ CFU/seed (4 strains) or as demonstrating poor colonization of soybean seeds when inoculated at 10⁶ CFU/seed (3 strains), were evaluated. Soybean seeds were treated at both 10⁵ and 10⁶ CFU/seed with each of the strains. Three repetitions were planted with 6 seeds per treatment per repetition.

Results demonstrate that inoculation at a target seed titer of 10⁵ CFU/seed can be used for identification of Methylobacterium strains with ability to colonize soybean shoots at a significantly higher density than control treatments and other strains previously shown to be poor colonizers of soybean shoots.

### Example 2 Soybean Phyllosphere Colonization Efficiency Screen

Methylobacterium strains that colonize the shoot surfaces of soy most densely when applied to seed at a dose of 10⁵ CFU/seed (a reduction from the 10⁶ CFU/seed levels used previously in field trials) were identified as follows.

Methylobacterium strains were tested for their ability to efficiently colonize soybean shoots. Soybean seeds were treated with Methylobacterium strains at a target seed titer of 10⁵ CFU/seed. Flo-Rite 1706 polymer was used to stick microbe to seed. Each experiment included 10 Methylobacterium strains, plus an untreated control treatment (UTC) and a strain that was shown in the past to have limited ability to colonize soybean phyllosphere (NLS0400, the “negative control”). In each experiment, two seeds per pot were planted in unamended field soil, with 20 pots per treatment level in a randomized complete block design, resulting in a total of 240 pots per experimental run. Plants were grown for 2 weeks in a greenhouse at 25° C. with regular watering and no fertilizer. At harvest, the two plants from each pot were cut at ˜1 cm above the soil surface and placed into a 50 mL conical tube with 15 mL of 0.9% saline solution. Ten pots per treatment were sampled. Each tube was weighed before and after plant sampling to quantify plant fresh weight. Samples were vortexed for 15 minutes, then placed into an ultrasonic bath for 10 minutes. Samples were then plated onto AMS-MC using an easySpiral automatic diluter and plater (Interscience, Inc.) at 5 dilutions, and plates were incubated for 8 days at 30° C. Plates were counted using a Scan 4000 automatic colony counter (Interscience, Inc.) to quantify the number of pink colonies. Results were recorded as the number of CFUs per mg of plant fresh weight.

Colonization density data (CFU/mg) were used to compare the strains in each run to the untreated control. A Mann-Whitney U-test was used to generate p-values comparing each treatment to the untreated control. The threshold for statistical significance used in this screen was p<0.05. Strains with significantly greater CFU/mg than the UTC were classified as “hits” based on which treatments were significantly higher than the negative control at p<0.05 using a Mann-Whitney U-test.

In some runs, the untreated control showed an unusually high value. NLS0400 was used as the standard for statistical comparison in any run in which 2 treatments or more showed mean CFU/mg lower than the UTC. Typically, hits colonized the shoot surfaces of soy at a rate that was 0.7-1.3 logs more CFUs per mg of plant fresh weight than the UTC or poorly-colonizing strains. Strains were considered poor colonizers and called “non-hits” if they displayed quantitatively lower colonization than the negative or untreated control. In one test, 380 individual strains were screened. Ninety strains were classified as hits, and sixty-five strains were classified as poor colonizers or “non-hits”.

### Example 3 Association Analysis

Genomes of the strains screened in the colonization efficiency were assembled and putative genes identified, then assigned a putative function by sequence analysis to databases of known genes and gene signatures. A pan-genome for Methylobacterium was constructed as described by Page et al. (Roary: rapid large-scale prokaryote pan genome analysis, Bioinformatics (2015) 31:3691-3693) except that genome sequences from greater than 1000 different species of Methylobacterium were assembled and used to construct the pan-genome as opposed to the single Salmonella species described by Page et al.

Strains that were identified as the most efficient colonizers or “hits” (as defined by p-value in the screen described above) and the poorest colonizers, “non-hits”, were compared to determine the presence or absence in each strain of each genetic element in the pan-genome. For this analysis, translated genes were clustered across strains using BLASTP with a sequence identity of at least 50% to identify homologous genetic elements across genomes. These results are used to determine which genetic elements are the same or different across strains, leading to a score for each genetic element as present or absent in a given strain. The presence/absence scores were used in a correlation analysis to identify genetic elements that correlate positively with colonization efficiency as described by Brynildsrud et al. (Rapid scoring of genes in microbial pan-genome-wide association studies with Scoary, Genome Biology (2016) 17:238).

The steps in the process are as follows. Correlated genetic elements were collapsed so that genes that are typically inherited together, for example genes on the same plasmid, were combined into a single unit. Each genetic element in the pan-genome received a null hypothesis of no association to the trait. A Fisher's exact test was performed on each genetic element with the assumption that all strains had a random and independently distributed probability for exhibiting each state, i.e. presence or absence of the genetic element. To control spurious associations due to population structure, the pairwise comparisons algorithm was applied using a phylogenetic tree of the Methylobacterium genus, constructed using the same genome sequences described above. Empirical p-value was computed using label-switching permutations, i.e. the test statistic was generated over random permutations of the phenotype data. The genetic elements that were significantly positively correlated with colonization efficiency were identified based on p value using a threshold for statistical significance of p less than or equal to 0.05. Sensitivity and specificity cutoffs were also employed. For sensitivity, i.e. using the presence of the gene as a determination of a strain as an efficient colonizer, a cutoff of greater than or equal to 25% was used. For specificity, i.e. using the non-presence of the gene as an indicator of a strain as a poor colonizer, a cutoff of greater than or equal to 75% was used.

Gene elements that are positively correlated with Methylobacterium colonization of soybean phyllosphere are shown in Table 2 below.

TABLE 2

| | Con- | | | | | |
|---|---|---|---|---|---|---|
| | sensus | Representative | | | | |
| | Protein | gene | | | | |
| | SEQ | sequences | | | | |
| Gene | ID | Nucleotide/ | | Sensi- | Speci- | p- |
| name | NO: | Protein | Annotation | tivity | ficity | value |
| cdhR_1 | 1 | SEQ ID NO: | hypothetical | 39.56 | 90.91 | 0.003 |
| | | 46/SEQ ID | protein | | | |
| | | NO: 91 | | | | |
| group_3 | 2 | SEQ ID NO: 47/ | hypothetical | 37.36 | 81.82 | 0.005 |
| 7721 | | SEQ ID NO: 92 | protein | | | |
| amaB_5 | 4 | SEQ ID NO: 48/ | N-carbamoyl- | 27.47 | 86.36 | 0.005 |
| | | SEQ ID NO: 93 | L-amino | | | |
| | | | acid hydrolase | | | |
| group_5 | 4 | SEQ ID NO: 49/ | hypothetical | 26.37 | 89.39 | 0.008 |
| 3552 | | SEQ ID NO: 94 | protein | | | |
| rbn | 5 | SEQ ID NO: 50/ | Ribonuclease | 26.37 | 93.94 | 0.016 |
| | | SEQ ID NO: 95 | BN | | | |
| group_6 | 6 | SEQ ID NO: 51/ | hypothetical | 38.46 | 78.79 | 0.018 |
| 798 | | SEQ ID NO: 96 | protein | | | |
| group_5 | 7 | SEQ ID NO: 52/ | hypothetical | 27.47 | 92.42 | 0.019 |
| 7813 | | SEQ ID NO: 97 | protein | | | |
| bbsG_1 | 8 | SEQ ID NO: 53/ | (R)-benzyl- | 41.76 | 84.85 | 0.022 |
| | | SEQ ID NO: 98 | succinyl-CoA | | | |
| | | | dehydro- | | | |
| | | | genase | | | |
| fecA | 9 | SEQ ID NO: 54/ | Vitamin B12 | 28.57 | 86.36 | 0.022 |
| | | SEQ ID NO: 99 | transporter | | | |
| | | | BtuB | | | |
| luxQ_3 | 10 | SEQ ID NO: 55/ | Autoinducer 2 | 30.77 | 84.85 | 0.028 |
| | | SEQ ID NO: | sensor kinase/ | | | |
| | | 100 | phosphatase | | | |
| | | | LuxQ | | | |
| group_7 | 11 | SEQ ID NO: 56/ | hypothetical | 38.46 | 86.36 | 0.028 |
| 164 | | SEQ ID NO: | protein | | | |
| | | 101 | | | | |
| mdlC | 12 | SEQ ID NO: 57/ | Benzoyl- | 45.05 | 77.27 | 0.029 |
| | | SEQ ID NO: | formate | | | |
| | | 102 | decarboxylase | | | |
| hddA | 13 | SEQ ID NO: 58/ | D-glycero- | 34.07 | 83.33 | 0.030 |
| | | SEQ ID NO: | alpha-D- | | | |
| | | 103 | manno- | | | |
| | | | heptose | | | |
| | | | 7-phosphate | | | |
| | | | kinase | | | |
| fptA | 14 | SEQ ID NO: 59/ | Fe(3+)- | 38.46 | 77.27 | 0.031 |
| | | SEQ ID NO: | pyochelin | | | |
| | | 104 | receptor | | | |
| livF_5 | 15 | SEQ ID NO: 60/ | High-affinity | 29.67 | 84.85 | 0.031 |
| | | SEQ ID NO: | branched- | | | |
| | | 105 | chain | | | |
| | | | amino acid | | | |
| | | | transport | | | |
| | | | ATP-binding | | | |
| | | | protein LivF | | | |
| group_4 | 16 | SEQ ID NO: 61/ | hypothetical | 28.57 | 92.42 | 0.031 |
| 5430 | | SEQ ID NO: | protein | | | |
| | | 106 | | | | |
| group_2 | 17 | SEQ ID NO: 62/ | hypothetical | 26.37 | 92.42 | 0.031 |
| 9067 | | SEQ ID NO: | protein | | | |
| | | 107 | | | | |
| hddC | 18 | SEQ ID NO: 63/ | D-glycero- | 30.77 | 84.85 | 0.032 |
| | | SEQ ID NO: | alpha- | | | |
| | | 108 | D-manno- | | | |
| | | | heptose 1- | | | |
| | | | phosphate | | | |
| | | | guanylyl- | | | |
| | | | transferase | | | |
| sutR_2 | 19 | SEQ ID NO: 64/ | HTH-type | 37.36 | 78.79 | 0.033 |
| | | SEQ ID NO: | transcriptional | | | |
| | | 109 | regulator | | | |
| | | | SutR | | | |
| ftsY | 20 | SEQ ID NO: 65/ | Signal | 40.66 | 78.79 | 0.033 |
| | | SEQ ID NO: | recognition | | | |
| | | 110 | particle | | | |
| | | | receptor FtsY | | | |
| bicA | 21 | SEQ ID NO: 66/ | C4- | 42.86 | 77.27 | 0.034 |
| | | SEQ ID NO: | dicarboxylic | | | |
| | | 111 | acid | | | |
| | | | transporter | | | |
| | | | DauA | | | |
| group_5 | 22 | SEQ ID NO: 67/ | hypothetical | 26.37 | 90.91 | 0.039 |
| 5945 | | SEQ ID NO: | protein | | | |
| | | 112 | | | | |
| livF_6 | 23 | SEQ ID NO: 68/ | hypothetical | 30.77 | 84.85 | 0.039 |
| | | SEQ ID NO: | protein | | | |
| | | 113 | | | | |
| hdhA | 24 | SEQ ID NO: 69/ | 7-alpha- | 46.15 | 81.82 | 0.040 |
| | | SEQ ID NO: | hydroxy- | | | |
| | | 114 | steroid | | | |
| | | | dehydro- | | | |
| | | | genase | | | |
| ssuA_7 | 25 | SEQ ID NO: 70/ | Putative | 31.87 | 84.85 | 0.041 |
| | | SEQ ID NO: | aliphatic | | | |
| | | 115 | sulfonates- | | | |
| | | | binding | | | |
| | | | protein | | | |
| uvrA_2 | 26 | SEQ ID NO: 71/ | UvrABC | 30.77 | 87.88 | 0.049 |
| | | SEQ ID NO: | system | | | |
| | | 116 | protein A | | | |
| group_1 | 27 | SEQ ID NO: 72/ | hypothetical | 48.35 | 83.33 | 0.051 |
| 9285 | | SEQ ID NO: | protein | | | |
| | | 117 | | | | |

Sequences of consensus proteins SEQ ID NO: 1 through SEQ ID NO:27 are provided below. In the consensus sequences, X can be any amino acid residue or can be absent.

SEQ ID NO: 1
XXXXXXXXXXXXXXXXXXXXXXXXXXXXXXXXXXXXMXXXXXXXXXXXXXX
XXXXXXXXXXXRRRXGXXMLLGTLAAGXXXXRPAXAEPVVEDVRIVDFD
WVDXARQRXVPARLYWPNTSSLRRXVPLVVFSHGLGQSRTGYSYLGRHWSSHGIAS
LHLQHVGSDSTVWTGNPLALLDRIDXAAXEREAIARARDLRFXLDRLLVQDGGXFG
DRIDXRRIVAAGHSYGANTTLIAAGARVIRDGXPLQXRDPRXXAGIVISAPPFYGERD
LRAVLXAVDIPTLHVTATEDXIQLPGRTXSPXXDRLXVYEAIXTPRKALAVFQGGSX
XXXXXXXXXXXXXXHSIFTDXXXXXRPLTGGXNLNPQVKXATAXGXLAFLDLAFR
GDPEPLXXXXXXXXXXXXRAWSSTWXXXXXXXXXXXXXXXXXXXXXXXXXXX
XXXXXXXXXXXXXXXXXXXXXXXXXXXXXXXXXXXXKPILAVAPAGFXXXSX
AQPAVAXRXRDRXXXXXXEPLXXXXXXXXXXXXXXXXXXXXXXXXXXXXXXXX
XXXXXXXXXXXXXXXXXX
SEQ ID NO: 2
XXXXXXXXXXXXXXXXXXXXXXXXXXXXXXXXXXXXXXXXXXXXXXXXMRK
KAXNRXXXXXXXXFTAXXXXXXXILXVAAXXXXGTXXXXLXLGAAEAXRDQXG
NYAQAEWAQDYASPAGMQVQRETXPILSPQTVAATEQMVERYRDIVNRGGWRPVS
GAERLRVGSKGPAVAAVRQRLIVTGDLDPAAGGSGVYDSYVAAGVKRFQARHGLS
QTGAMSMATQQAMNVPADVRLXQLETNVVRLRSYSGDLGRRFVITNIPAALVETVE
NGQVVTLHAAGVGKIDRQSPIMNTKATQINFNPTWTVPASIVKKDLIPKMQKDPNYL
TDNKIRILSGXXEISPRSVNWNSDEGTRYTYRQDSGADENSMGIVRINIPNPYGVFMH
DTNTKGVFGDDFRFISSGCVRVQNVREYITWLLKDTPGWXREQVEQAIESGKRVDA
NIAQPVPVYWTYITAWATPDGLVQFRDDIYKRDGVNVPSTIGAPTPVASAEPLXPQT
FEPGDXEEXXXX
SEQ ID NO: 3
XXXXXXXXXXXXXXXXXXXXXXXXXXXXXXXXXXXXXXXXXXXXXXXXXXX
MTEIDTDAFLADLYALREIGRFRTGVHRPTFSAXDMESRRWLMAKLEXCGLEASIDG
IGNVLGRHRGPGPHLLVGSHIETQNEAGWLDGALGVVAGLALARAGLPVDVVAFA
DEEGHFSGGFLGSRSAIGDLTEAEIDAARNRTDGTPLRAALESAGLAGLPRMRLDPA
RYRGFLELHIEQGTQLESAGLHLGVVSGIVAIWQFQIVFDGNQDHAGGTTMAERRDA
GLSAVRLLAAIDREFPKVCGPRSTWTTGRITLDPGGYSIIPGRAEVAFQFRDVSMPVL
ERMEACLEALVRESNRRERCPATLTALSKAIPAPCDPDLMRALSEAAEQVCPGRWQ
VMPSGAGHDAQNIARILPAAMLFVPSIGGISHHWAEDTSDADLAFGVRALGAAAAR
VLAGXXXXXXXXXXX
SEQ ID NO: 4
XXXMRRPDQPALTAAAHGRVSMPFLPTLAVVAILGVASLYCPTSKPGQPXGARVEX
XXXXXXXXXXXXXAGVSAAPGPTDFAPIAAXVAAPGRPPAVIAFAEQYPLDAXVIA
RTGSLPARPAVAARANAHVAAAGRRACPGRRCPETPRSNTDPMAPARGXAADEAE
DALLPSQAMPFAASVVETLVPAARAVGDAANLVRSSARAVQGTVALAVADCLR
SEQ ID NO: 5
MSNSSEPTILAADQDLALRFWGVRGSTPVSGPQYAEFGGSTPCIEVRCGQRMFIVDA
GSGIYNLGQGHRTDLPQEVDLLFSHLHLDHTAGLPFFKPAVLDPDRVINTYCGNLGG
ESAGPTLDRLFAPPLFPVTLDKLCCTFHHHGFEAGQTLAFPDGTRVATILLNHPQGSV
GYRFEHGGRRLCLISDIEHSDPWPDPELAAFVADADLMVYDGMFTDGEYPTCRGWG
HSTWQKGVELARNAGVKALGIIHLHPAHSDTALRDMEADLQAEMPTAFIARERQSLI
VGAPRAVSGRPSGRPAVAREMRRRIKVA
SEQ ID NO: 6
XXXXXXXXXXXXXXXXXXXXXXXXXXXXXXXXXXXXXXXXXXXXXXXXXXXX
XXXXXXXXXXXXXXXXXXXXXXXXXXXXXXXXXXXXXXXXXXXXXXXXXXXX
XXXXXXXXXXXXXXXXXXXXXXXXXXXXXXXXXXXXXXXXXMXXXXXXXXX
XXXXXXXXXXXXXXXXXXXXXXXXXXXXXXXXXXXXXXXXXXXXXXXXXXXX
XXXXXXXXXXXXXXXXXXXXXXXXXXXXXXXXXXXXXXXXXXXXXXXXXXXX
XXXXXXXXXXXXXXXXXXXXXXXSVXIXXXXXXXXXXXXXXXXXXXXXXXXX
XXXXXXXXXXXXXXXXXXXXXXXXXXXXXXXXXXXXXXXXXXXXXXXXXXXX
XXXXXXXXXXXXXXXXXXXXXXXLXXXXPXXXMRIVXXXXXXAAXXXXXXXX
XXXXXXXXXXXXXXXXXXXXXXXAAXXXXXAPXXXXXXAAXXVKAGDLXIETP
WLRATPGGAKVAGGYVRIXNTGSXPDRLTGASIPXAGRGEIHSMSMEGGVMKMAP
VXGGLXIKPGETVELKPGGYHLMFEDLTXAPKAGETVSGTLTFERAGTVPVTFTVAP
IGAXAXPXXXXXXXXXXXXXXXXXXXXXXXXXXXGHQHXHHXXXX
SEQ ID NO: 7
MXXXXXXXXXRSAGDAXSASLMDQIAPARTGRTRRRPHPALVVASVALAIGLWFVI
YAVAGRAIWFW
SEQ ID NO: 8
XXXXXXXXXXXXXXXXXXXXXXVDFTLSXSQTHWLTRVRAFIADAILPAAAAVAA
ERAQSRXPSPTMERLKDKARXEGLWNLFLPPSPEHDTDEYRGAGLTNLDYALCAEE
MGRVGIASEVFNCAAPDTGNMEVLHRYGTXAQKDRWLKPLMAGEIRSAFLMTEPE
VASSDATNISTSIRRDGDHYVIDGRKWWSTGVGDPRCRIAILMGKTDPEAPRHRQQS
QILVPMDTPGVRVERLLPVFGYEDAPKGHGEVVLENVRVPAGNLILGEGRGFEIAQG
RLGPGRIHHCMRTIGAAEVALEAMARRLVSRVAFGKRISEQSVWEQRVAEARIDIEM
TRLLCLKAADMMDKVGNKGAKLEIAMIKVAAPRVALKVIDDAIQAHGGAGVSEDF
GLARMYAHIRTLRLADGPDEVHNRSIARLEFGRYTNARXGXXXXXXX
SEQ ID NO: 9
XXXXXXXXXXXXXXXXMPRXFRXGXXXRAALLXXXXLXXXATPAAAQXXXXXX
XSAASVTLXELSVXGAAAXXXXXXPXXXEXXGSLTVPSVXRQRAALNXTVGSVAF
VDAXXXQDRYANTLRDVLKDVPGVYVQERYGQELRLSVRGSGIARGFHLRGLELL
QDGIPLNLADGSGDFYQVDPLALRSVEVYKGGNALTFGATTLGGAVNXVTPTAYTA
LAPNILRVDGGSFGTIRENFQMSRIXGPLDXLVNGTLTNSDGFRXHEAQRTQNFNANI
GYRIAPGIETRFYLGXYLTDQKLPGTLTLGQSLSTPTLANPTAITGNQSRKVETERIAN
RTSFLLDVGKLDIDTWAIHKSLYHPIFQVIDQDGWTYGISPHWAGTFDIGGFRNDTIL
GLRAFAGQNSALQFVNVRGORGAQTLNALQSASNXEAYGENRFWFLPDVALMTGA
KAFSSNRTYSDKGGXPXGNPXXRFADVTYEGVNPKIGLLWQPXPDIQVFGDVTRSR
DVPDFSDLVQQNLLSTTFVPLRAQRAWTYEAGARGRIDRLAWDVTLYRSDLRDELI
NFSTNPGXXLNIPAATFNTPRTVHQGVEAAVTLDLARDLXGXGDGLSVTQIWTHND
FRFVGDPVFGNNRIAGIPXDVLRTVMSYRHPSGFHIAPSLDWVPQGAFADHANTLRV
PGYALLGVEAGIDFANGVSLFVDARNLTDARYVSDIAVVANAXXXXXXXAATAGG
PXALAAFYPGSGRSVFXXXXXXXXXXXXXXXXXGGIRXXXXXXXXXXXXXXXXX
XXXXXXXXASFXXXXXXXXXXXXXXXXXXXXXXXXXXX
SEQ ID NO: 10
XXXXXXMSETESGPGSVTDADYRNLAETLPQLAWIAEADGTIVWYNQRWYDYTGT
SLDEMRGWGWRTVHHPDHVAAVTERYRAAITLGRSWEDTFPLRGRDGSYRWFLSK
ALPHRDESGRILRWYGTNTDITVRRAVEERLRHSEQRFRALVDASAAVIWNTDAAG
ELMPPQVRWSTYTGQTEEAYQGWGWLDAVHPDDRGHAADAWAACVEARATYEV
EYRLRRHDGAWRAMEVRGVPVLAEDGSLREWVGTCVDVTERKEAEEAVERARQA
AEAANRAKSQFIANMSHELRTPLSAVIGYSEMLGEELEDIGQAALLPDLRKIEAAARH
LLSLINDVLDISKIEAGRMTASAETFTVADLLRDVTDSTGSLVEKKGNRFVLDAGAA
GEAGLGSMHQDQTKIRQCLLNLIGNAAKFTERGTITLTVRRHREAGADWLSFAVAD
TGIGLTEAQIDRLFERFVQADDSTTRQFGGTGLGLAITRAFCRTMGGDIGVASTPGAG
ATFTIRLPATLRPEDAXPPXXXTEAEAHTPVEPHEEHETVLLVDDDPAARELLQRFLE
REGFHVRSANDGRAGLTLARALKPRAILLDVEMPRMDGWAVLHAVRNDPDLAGTP
VIMTSVVAEQGLGQALGATDYFVKPIDWDRLKGLMERYRPAAPNEARVLVVDDDA
DARERLRRSLGREGWTVDEAENGRIALERVSQARPSLILLDLMMPEMDGFGFLRALR
SRPDGDVPVVVLTAKEVTAAEKESLNRQADRVIAKGSMSLAEIGRQLRVLYARSATE
PVPGQLQGLLDRLAEKDAGEPRXXXXXXXXXX
SEQ ID NO: 11
MMMHVKMIAAAAAVVGGLGLAXSAGAAPLAPAGADTITGPAAVSTVAFGCGPGW
APGPYGRCRPIXXXYRRPRFYGPRCFFRPTPWGXXXXXXXXXXXXXXXPRRVCRXX
SEQ ID NO: 12
MXIETVETAGRRRGADLLVEVLRSEGVRYIFGNPGTTELPLIDALTEAPDIAYILALQE
ATAVAMADGYAQGARRPAFLNLHTAGGLGHXMGGLVNSQVSGTPLVVTAGQQDL
RHALTDPLLMGDLVAIADPVMKWAREVTSPDQIPILLRRAFHDXGAAPSGPVFLSLP
MDVMEALTAVPAGETSTIDQRAVAGSLDRLAEKLAAIAPGRLALIAGDEIDASDASA
QMVALADLLAAPVYGSSWPAHIPFPTAHPLWAGNLPTRADAIADILGRYDAVFALG
GKSLITVLYSEVSAVPPGVQVFQLSADVRDLGRTYATXLSTVGDIRASLDALLPLLAP
RLADRADAFAXLRAQAVTARAERRAKLAAAADAAFEDPVIAPLVAAREVARAVGA
ETTIVDEAPATLTHLRTFLDSPSAHQYAAMRGGVLGWGMPAAVGFSLGLDRAPVVC
VVGDGAAMYSPQALWTAAHEKLPVTFVVINNAEYNILKTFMKGQAHYASVRANRF
IAMDLTDPRIDFPALAASMGVPARRVTRAADIAPAIEAGIRSGGANLVEVVVRAT
SEQ ID NO: 13
MLTVTRTPLRISLFGGGTDYPEYFERAPGAVVGAAIDKYIIIAALDLIGCQDYNYRLS
YSRVEHCNNIXEIEHPVVREVLKHFDVNRRLDMSIISDLPAAGSGLGSSSAFTVGFLR
TIYAILNQKPTKIELSKKAIEVEREILRENVGVQDQLHAAFGGINRFDFSGSAIRISPVQ
MSSAAIQQLNASMVLVHTGIARRATTTVAAQIAVTRARAIDKELSELYRLVEECVSL
LEAGTSGWLXQLGEMLSASWRIKRTLSREVSNAVLDDLFEAIIASGAYGAKLCGAG
GGGFFLALIDPDRLPALXERVAPLSVVPIGIDVDGSTLIYRQTRXXXXXX
SEQ ID NO: 14
MSLRALRSSLVASASVLAGTVLPXAVQAQQSVTLGEISVVSTSPVGSGGGNSSXQAQ
IFNGPGPVPPAGSLGPSXXGVRLRGSEQPLYKIPSTVESVTASDITIDRASDNLTTTLAR
RTPGINVSDSQGNNNRVDITYRGFTASPVQGVPQGLAVYQNGVRINEAFGDIVNFDLI
PPQAIQRIDVVTGNPVFGLNALGGAVNIQMKNGFTWQGTEISAWGGSDARTAGYLE
YGKVSGPWSVYFTGDGLNDRGWRYESPSTIGRLYGDIGYRSQDSEFHLIGLAARSFF
GAAAATPVDFTHRDPRAIFTYPQTTTTEVGTLQLTGRVDISPTWDLAGNAYFRRFSQ
TYVDGNDGNFENCSTRSSFRGNLCFEDDGFSPAAGQSQLAFRNQFLILGQXXQNQRI
PFRAGIPYGTLDTTRTEATGFGGSLQAANRDRIFGLSNTFVVGGSIDAANYXFKSSST
LGVINPDLSITTDPSNPFYGNIPGLGTPQLRTAGALGIAPSSVNGSNLYMGLYTLDTLD
VTDRLSLTAGXXXXARLNFARIQSEDLTGFSPDVTGTHYFNKINPVAGLTYRFFDAL
NLYGSYSESNRAPTPLELACANPDRPCLLPNSLVADPPLKQVTGRTYEVGFRGQLPN
TYDGGIITYKIGAFRTDLANDILSLATPGNTARAYFVNVPSTQRQGIEVGGEYXXTAD
YLRVYANYALVDATFQFNGTLSSPNNPLAXXXXXXDDGAIQVRKGNVVPLVPTHQ
XKAGFDYFVTPNWQFGLYLQAFSSSYFRGDESNLNRKLPPYYVLNFQTKYQVTKNL
EVFGLITNLTNNRYXTFGTFAEPGAVAGNLRISDPRTTTLAQPFSVYAGIRYAFGADP
VPMSXPEPIIRKY
SEQ ID NO: 15
XXXXXXXXXXXXMLEIDRLDAWYGPSHVLHGLSLEVRPGEILALVGRNGAGKTTT
MKAVMGLIPKVAGSVRFLGEDLLGRPXHARFPXGLAYVPEDRRIVPGLTVRENLKL
GLLRAXXXXXXXXXXXXXXXXXAXIKEGPAIAEIAETFPRLAERLDQTAVTMSGGE
QQMLAIARAMIARPKMILLDEPSEGIMPVLVEEMGRLFVAMRXRGVTLLL VEQNVE
WALNLADRAXIIDQGAVVHXSXAAALRADXEIQDRYCAV
SEQ ID NO: 16
MNFVHRYLGESRAVSDADATRLGFVPDTLREPTYFAGTVARHLPFREAISALHHVVV
SDLRFKPKDRTAYFAWLQAHEQELLAEALAEKDSLRAEIEALRAESRDIAARSDAVM
RSFYDARKRYFDYLYRENLDAWIVLDPVITVHPDEIFFEAFSLDESSYGRLSCDHDTF
ARIGDMACGTTNIDYSHALYDEFQKIRSYRDTELAIDPTGFAVQTSGEAAYREDKIDL
PDSWMRGFLQVSSAMAQPAHVVDLHPVDMHAILTRLAARRERHGPRSLRFLLEPGR
PVSVLIEPWNERLTFRRSIYRGGEXAEIRLWGRRRLAILARTLPLARSVRLHLLGTGLP
SFAVVDFGGLRFTLGLSGWTANDWSRAGQFDLLAPRADVDADTAARVFAALRRHH
AADTGQLAAETGLDRSTVEAALGGYVQAGRAMFDLDKRVYRLRELTREPLAPGAL
RFASEQEAKADRFLAAGLVTLGPVEQAGDRRRLSGTVLDDGRSLTPAVELDSDDRM
VGGSCQCGFYTHNRLTRGPCEHMLAVRRLVHAQVEGKPVRWQA
SEQ ID NO: 17
MADPAPITGAPEPVKAAPGAFLTPLFTDRRVAAVLGLGFAQGIPFLLVYATQSAWLV
QAKVPLATIGLMSELTIAYKLKFLWAPFLDRHDAPLIGRWLGRRRGWIVATQILVAL
ALAGVAFGDPAHWLAWTVAFSLALGVAGATQDVVIDGWRITAAPPEQQALMSSWA
EIGFRIGNLAAGAGALYLSDAYGWRVAYLCMAALMAPGTVAALLAPEPPVPETPAT
XXXXXXXXXXXXXXXXXXXXXXXXXXXXXXXGGFVETVWAPIRDLLAXXXRLG
PLALPVLALVAGFRMPGYVSNAMAIPLFKTLGYTNTDIATVTKLFGFWIALGGTFLA
SAIIPRIGMMASLLIGTVTASASHLALAYLAWHGGHGGAAFWTFALTVGIDGFAYAF
ASIVLITYMSRLSATAHAASQYALLTSLCALPGSLLAGFSGFVIEWTGFAWFFVGTSLI
GVPVALLCLLVARRHGPMEPAADAAGDAPNRAT
SEQ ID NO: 18
MSDPSPAVSRAFVLAAGLGKRMRPVTATVPKPLVEVAGKALLDHALDRVAEAGIGT
AIVNVHYLXDLIEGHLARRAEGXAXGPATTVSDERAALLETGGGIRKALPLLGDAPF
VVLNSDSFWLEGPAXNLRRLIDTWDGDRMDGLLLVAPTATSLGYEGAGDFVMDPD
GRLERRGERAVAPFIYAGVAILTPGLFADTPEGAFSLNLLFDRAIAAGRLFGMRLDGQ
WLHVGTPDAIRAAEERVRASARAP
SEQ ID NO: 19
XXXXXXXXXXXXXXXXXXXXXXXXXXXXXXXXXXXXXXXXXXXXXXXXXXXX
XXXXXXXXXXXXXMTVRTIIXXXGRDPAAAETSPQIHIXAEEIRRRRREQGLSLEXL
AARSGVSRSMISKIERSEAVPSTVVLSRLAEALGVTFSRLMAPATEREVLLIPASRQPI
LRDEASGYLRRCISPVLPGRGIDWVLNTLPPGASTGEFTAHRRGVSEYIYVLRGRLRA
VIGERAVVMEXGDSLYFEADAGHAFTNVGTEACEYFLVIDPSRVRXXXX
SEQ ID NO: 20
MSDDEKPGWFGRLFGRKXXXXGAPESKSXEPVPAEDXXXXXXXEEXPXXXXXADE
SVTPDPLSPXASESEGQPXFTTAADDVAHVPPPVSEPAPDDPDKNRIPDELEGADLQP
XXXXEPQPPSGXXXXEAPQPDEXXXXXXXXXESXXEXXAXXXEXAEKRNWWSRL
TGGEXEXXXXXXXPAPAXXXXXXXXXXXXXXXXXXXXXXXAESEPPAEVDIQPV
DSAAALASDAVSGAVEGSEKQGWWSRLTAGMRRTSSALSDRVTGLFTKRKLDATT
LEDLEDALIQADFGVETATRMSEAVGKGRYEKGISPDEVRAILATEIERALEPVALPIE
IDSAKKPYVILTVGVNGAGKTTTIGKLSLKFKAEGRSVMLAAGDTFRAAAIEQLRVW
GDRTGTPVISRAQGSDAAGLAFDAFKEARENGTDVLLIDTAGRLQNKAGLMAELEKI
VRVIRKLDPEAPHATLLVLDATVGQNALSQVELFSQAAPVSGLVMTKLDGTARGGIL
VALATKFGLPVHFIGVGEGVEDLEPFAARDFARAITGLPKEXXX
SEQ ID NO: 21
XXXXXXXMRASLARLMPXXXXXXXXXSTLGRDLPASFVVFLVAMPLCMGIAMAS
GVPAERGLITGIIGGIVVGFLAGSPLQVSGPAAGLAVIVFEFVREHGIDALGPVLVAAG
AIQLLAGALRVGGWFRAISPAVVHGMLAGIGILIVLAQIHVLTDALPKASGLDNLVAI
PAAFFNFVSGPDGNRPGAVIVGLATIVAMIGWEKIRPAKLKLIPGALMGVLAGTLVA
VVGSMDVKRVEVPENIFSAVTMPGXGDWSRLAEPAMILMAVTLA VIASAESLLSAA
AVDRMHDGPRTQYNRELGAQGIGNILCGLAGGLPMTGVIVRSSANVQAGAATRAST
ILHGSWILAFLLVLPMVLKVVPTASLAGILVVTGWRLVSPAHAFHLHERYGLPTAAI
WLATMVMVVATDLLTGVLTGLALSLLQVIPHXYLRGPLKIEGGAAQTVQGGAIQAV
PELRLSGSATFLQLPHLNAALEXRTPEGSPVRLAAQDLRHVDHTCLEMIREWATRRA
KTGSRVEVVGGGASGLHHSXLAMVAHAAXXXPKD
SEQ ID NO: 22
MIVLIAAAIVSGLATATILAPVSALAALIIAPLAASASAILACIFIAWRNTRDDVGPPDL
ETQADAMVAVLCEVAQQGKIVPVAAPVRVRGHRSAXXXX
SEQ ID NO 23
XXXXXXXXXXXXXXXXXXXXXXXXXXXXXXXXXXXXXXXXXXXXMDITGEYRI
XAPRAAVWAALNXXXXXXXXXXXXXXDPEVLAXXXXXXXXXXXXXXXXXXRCIP
GCKELTQASPEEMAAKVALKVGPVSATFAGTVXRFEDIRXPEGYTLVGQGNGGMA
GFAKXXXGRAXVSLREEGXETVLTYEAKAEVGGKIASLGGRXXXXXLIQXTXXXSR
KLADQFFGXFAAELGAPXPXAAAXAAXXXXXXXXXXXXX
SEQ ID NO: 24
XXXXXXXXXXMSLFDLTGKXALITGSSRGIGRAIALRMAEHGARVVISSRKREACEA
VVAEIEAAHGAGRAVAIPASISVKXELEXLVXETESRLGPVDVLVCNAASNPYYGPL
AGISDAQFRKILENNVLSNHWLIQMVAPGMVARRDGAIVIVSSIGALKGSPVIGAYN
VSKAADLQLARNYAVEYGXXXXXPANVRVNCLCPGLIRTDFARALWEDPEMLAAT
TDAAPLRRIGEPDEIAGAAVFLASAAGRFVTGQALVIDGGVTIAXXXXXXXXXXXX
XXXXXXXXXXXXXXXXXXXXXXXXXXXXXXXXXXXXXXXXXXXXXXXXXXXX
XXXXXXXXXXXXXXXXXXXXXXXXXXXXXXXXXXXXXXXXXXXXXXXXXXXX
XXXXXXXXXXXXXXXXXXXXXXXXXXXXXXXXXXXXXXXXXXXXXXXXXXXX
XXXXXXXXXXXXXXXXXXXXXXXXXXXXXXXXXXXXXXXXXXXXXXXXXXXX
XXXXXXXXXXXXXXXXXXXXXXXXXXXXXXXXXXXXXXXXXXXXXXXXXXXX
XXXXXXXXXXXXXXXXXXXXXXXXXXXXXXXXXXXXXXXXXXXXXXXXXXXX
XXXXXXXXXXXXXXXXXXXXXXXXXXXXXXXXXXXXXXXXXXXXXXXXXXXX
XXXXXXXXXXXXXXXXXXXXXXXXXXXXXXXXXXXXXXXXXXXXXXXXXXXX
XXXXXXXXXXXXXXXXXXXXXXXXXXXXXXXXXXXXXXXXXXXXXXXXXXXX
XXXXXXXXXXXXXXXXXXXXXXXXXXXXXXXXXXXXXXXXXXXXXXXXXXXX
XXXXXXXXXXXXXXXXXXXXXXXXXXXXXR
SEQ ID NO: 25
XXXXXXXXXXXXXXXXXXXXXXXXXXXXXXXXXXXXXXXXXXXXMXIRSXAXR
LTRRRAXAXLAAALALXXXXAXXXXXXXXXXXXXXAAPSXAAEPVVLRVGDQKG
GNRSLLEISGYGKDLPYTIAWSEFPAAAPILEALNAGALDVGYTGDLSLLTAYAAGA
PIKAIGGTKSDPRTQAILVRQDSPIRSAADLKGKRLAGTRGGWGQFLIDATLEKAGIK
XEDATFAXLNPVDAKVALVAGSVDAWAVWEPYVSFATLKDKARXIADGXGLTPTI
TFIVASDSAIATKRAAVQDXLERLNKARLWSXXXLDHLDXYARNTAALTRMPEDVL
RAAYTAQRTSPIGIDDGVVQEVQAASDRATRYGILXKRLDVXRVLDRSFTAAAXNXX
SEQ ID NO: 26
XXXXXXXXXXXXXXXXXXXXXXXXXXXXXXXXXXXXXXXXXXXXMAKXXXX
XXXXXXXXXXXXXXXXXXXXXXXXXXXXXXXXXXXXXXXXXXXKARKPETAX
XXXAKXXXXXXXXXXXXXXXXXXXXXXXXXXSKGRAEERTDAQLSALFDAAAX
XXPAARDTRVISVRGAREHNLKNVDLTIPRDRFVVFTGLSGSGKSSLAFDTIYAEGQR
RYVESLSAYARQFLEMMSKPDVDQIDGLSPAISIEQKTTSKNPRSTVGTVTEIYDYMR
LLWARVGIPYSPATGEPIESQTVSQMVDRVLELPEKTRLYLLAPVVRGRKGEYRKEI
AEFQKKGFQRLRIDGEYYAIDDVPKLDKKLKHDIDVVVDRIVVRDDIAARLADSFET
ALELADGIADIEFADAPEGEAPKKITFSSRFACPVSGFTIPEIEPRLFSFNNPFGACPTCG
GIGHEMRIDPELVISDSALTLKRGAVGPWAKSTSPYYDQTLDALAKHFGFKTSVAWS
ALPEQAREVILFXXXXXXXXXXXGTGKESVRFDYNDGLRSYSVNKPFEGVIPNLERR
YKETESDASREEIGRFMSATPCAACDGKRLKPEALAVKIDRQDIGQVTALSVREXXX
XXXXXXXXXXXXXXXXXXXXXXXXXXXXXXXXXAHRWFSEISGXXXXXXXXXX
XKLTDKQNEIAVRILKEIRDRLTFLVDVGLEYLTLARGSGSLSGGESQRIRLASQIGSG
LTGVLYVLDEPSIGLHQRDNERLLGTLKRLRDLGNSVIVVEHDEDAILQADYVVDVG
PGAGIHGGEIVAQGTPEELLKDPASLTAKYLTGELSVRTPKARRKPGRGMLRLVGAR
GHNLKNVTAEIPLGTFTCISGVSGGGKSTLIIDTLYKAAAKRLNGALEXXXXXXXXX
XXXXHPAPFERIEGLEHLDKVIDIDQSPIGRTPRSNPATYTGAFTPIRDWFAGLPEAKA
RGYQAGRFSFNVKGGRCEACSGDGVIKIEMHELPDVYVTCDVCKGKRYDRETLEVR
YRNKSIADVLDMTVEEAADLFKAVPSIREKMETLARVGLHYVRVGQQATTLSGGEA
QRVKLSKELSKRATGRTLYILDEPTTGLHFHDVAKLMEVLHELVDQGNTVVVIEHNL
EVIKTADWVIDMGPEGGDGGGRVVAQGTPEEIAASTASHTGRFLREVLARXXXXXX
RPAGKAVKXXXXDAAKDXXXXXXXXXXXXXXXXXXXXXXKAKAGAASGRRSNA
AXXGRQAAE
SEQ ID NO: 27
XXXXXXXXXXXXXXXXXXXXXXXXXXXXXXXXXXXXXXXXXXXXXXAXAXL
XAXXXALAXSXLXXXIGXXPAEARPAGYEGSAPSCFLTGGGALEICRDGSEAEGVLI
APAPCAFVRGVQILRLARSEAPGPLQAVFQGQTPVASXRRDEPCPERRATDSRDGGA
LRTGRTFEALVTTLPPSADRFWVDLPEERARXRGWSEPTPSAEGIGPRDPIAVTGRTW
AGEAYSYVFLLGQERVGVTGGXDAEPERRGRRRRRRAXXXEXDAPATGPADVRRR
VQIEARTLDFQTFEIRTRTADGYGLAWTPFEAXXXXGAGRGRRXDPPXPAAVVDEA
GKPVTSACTAPAFETHGLAGSISVVDRTYHYVYTDVLPEDCGLAPEKRRTGLFLRTA
QDLSGPKVWSTARKLAGPLPPGTLVRVARAKGMQRWAVSYTCQRPANAPGGPVAD
ICLQYTADLNLDGIGALKLYADPVEAGRSXAYLGLRSGGDGSGRYDRSAHFWMTD
AEGNLDTPAIYPNKAGFLTWLDRLAPTASGRDASSLYGRPVYWATWTVRXIGAQ

### Example 4 Soybean Root and Shoot Colonization Efficiency Evaluation

Six Methylobacterium strains were tested for their ability to efficiently colonize soybean roots as follows. Soybean seeds were treated with Methylobacterium strains at a target seed titer of 10⁵ CFU/seed. Flo-Rite 1706 polymer was used to stick microbe to seed. Seeds were planted in unamended field soil using a randomized complete block design. Plants were grown for 14 days in a greenhouse at 25° C. with regular watering and no fertilizer, after which, roots were harvested. During harvest, the bulk soil (soil that is not physically adhered to the root surface) was shaken off. The root system was then placed in 15 mL of a 0.9% saline solution in a 50 mL conical tube and vortexed for 5 minutes to remove surface-adhered soil. The root system was then removed and placed in a fresh 50 mL tube containing 15 mL of 0.9% saline solution, vortexed for 15 minutes and placed in an ultrasonic bath for 10 minutes. The roots were then removed and the tube contents were poured into the previous tube containing soil. The soil and Methylobacterium were centrifuged into a pellet, the supernatant removed, and the soil pellet allowed to dry completely over a period of 96 hours,

The dry soil was sampled to determine the colonization density of Methylobacterium using qPCR primers specifically validated for the target strains using a soil PCR kit. Colonization density was recorded as copy number per gram of tissue.

The same six Methylobacterium strains were tested for their ability to efficiently colonize soybean shoots. Soybean seeds were treated with Methylobacterium strains at a target seed titer of 10⁵ CFU/seed as described in Example 2 above. Colonization density of Methylobacterium on the shoots was determined using qPCR primers specifically validated for the tested strains and recorded as copy number per gram of tissue,

Results demonstrate that qPCR using specific probes can be used to assess colonization density in a colonization screen and differentiate between efficient and poor colonizers. Results also demonstrate that Methylobacterium strains identified as efficient colonizers of soybean shoots also substantially colonize soy root surfaces.

### Example 5 Corn Root Colonization Efficiency Screens

### Root and Rhizosphere Screen Using PCR Quantification

Methylobacterium strains are tested for their ability to efficiently colonize corn roots or associated rhizosphere as follows. Corn seeds are treated with Methylobacterium strains at a target seed titer of 10⁵ CFU/seed. Flo-Rite 1706 polymer is used to stick microbe to seed. Each experiment includes multiple Methylobacterium strains, plus an untreated control treatment (UTC). A strain shown in the past to have limited ability to colonize corn roots may be included as an additional negative control. Corn seeds are planted in unamended field soil, using a randomized complete block design. Plants are grown for 14 days in a greenhouse at 25° C. with regular watering and no fertilizer, after which, roots are harvested. During harvest, the bulk soil (soil that is not physically adhered to the root surface) is shaken off. The root system is then placed in 15 mL of a 0.9% saline solution in a 50 mL conical tube and vortexed for 5 minutes to remove surface-adhered soil. The root system is then removed and placed in a fresh 50 mL tube containing 15 mL of 0.9% saline solution, vortexed for 15 minutes and placed in an ultrasonic bath for 10 minutes. The roots are then removed, and the tube contents poured into the previous tube containing soil. The soil and Methylobacterium are centrifuged into a pellet, the supernatant removed, and the soil pellet allowed to dry completely over a period of 96 hours.

The dry soil is sampled to determine the quantity of Methylobacterium using qPCR primers specifically validated for the target strains using a soil PCR kit. Colonization density is recorded as copy number per gram of tissue.

### Corn Root Colonization Assay Using Non-Soil Media

Methylobacterium strains were tested for their ability to efficiently colonize corn roots on corn seedlings grown in non-soil media. The experiment included multiple Methylobacterium strains, plus an untreated control treatment (UTC). Corn seeds were planted in calcined clay media, Turface, using a randomized complete block design. The seed was inoculated by micropipette with a 100 microliter suspension of the M-troph isolate in culture media at a concentration of 10⁵ CFU/seed. The untreated control received 100 microliters of culture media only.

Plants were grown for 14 days in a greenhouse at 25° C. with regular watering and no fertilizer, after which, roots were harvested. During harvest, the calcined clay was removed from the roots by shaking. The root system was then placed in 15 mL of phosphate-buffered saline solution and vortexed and sonicated to remove cells. The root wash was then plated on selective media and the number of Methylobacterium colonies, as identified by their pink color on the plates, was determined.

Strains that were identified as the most efficient colonizers or “hits” (as defined by p-value in the screen described above) and the poorest colonizers, “non-hits”, were analyzed to identify genes associated with enhanced colonization as described above. Gene elements that are positively correlated with Methylobacterium colonization of corn roots grown in non-soil media are shown in Table 3 below.

TABLE 3

| | Con- | | | | | |
|---|---|---|---|---|---|---|
| | sensus | | | | | |
| | Pro- | Representative | | | | |
| | tein | gene | | | | |
| | SEQ | sequences | | | | |
| Gene | ID | Nucleotide/ | | Sensi- | Speci- | p- |
| name | NO: | Protein | Annotation | tivity | ficity | value |
| group_ | 28 | SEQ ID NO: 73/ | hypothetical | 35.29 | 95.24 | 0.029 |
| 13482 | | SEQ ID NO: 118 | protein | | | |
| gpmA_ | 29 | SEQ ID NO: 74/ | 2,3-bisphos- | 47.06 | 90.48 | 0.038 |
| 2 | | SEQ ID NO: 119 | phoglycerate- | | | |
| | | | dependent | | | |
| | | | phospho- | | | |
| | | | glycerate | | | |
| | | | mutase | | | |
| ecfG_1 | 30 | SEQ ID NO: 75/ | ECF RNA | 47.06 | 90.48 | 0.052 |
| | | SEQ ID NO: 120 | polymerase | | | |
| | | | sigma | | | |
| | | | factor EcfG | | | |
| group_ | 31 | SEQ ID NO: 76/ | hypothetical | 29.41 | 100.00 | 0.069 |
| 47285 | | SEQ ID NO: 121 | protein | | | |
| group_ | 32 | SEQ ID NO: 77/ | hypothetical | 29.41 | 100.00 | 0.069 |
| 59039 | | SEQ ID NO: 122 | protein | | | |
| group_ | 33 | SEQ ID NO: 78/ | hypothetical | 41.18 | 95.24 | 0.081 |
| 35386 | | SEQ ID NO: 123 | protein | | | |
| adh | 34 | SEQ ID NO: 79/ | putative | 47.06 | 90.48 | 0.082 |
| | | SEQ ID NO: 124 | zinc- | | | |
| | | | binding | | | |
| | | | alcohol | | | |
| | | | dehydro- | | | |
| | | | genase | | | |
| group_ | 35 | SEQ ID NO: 80/ | hypothetical | 47.06 | 90.48 | 0.082 |
| 53489 | | SEQ ID NO: 125 | protein | | | |
| lgt | 36 | SEQ ID NO: 81/ | Prolipo- | 47.06 | 85.71 | 0.082 |
| | | SEQ ID NO: 126 | protein | | | |
| | | | diacyl- | | | |
| | | | glyceryl | | | |
| | | | transferase | | | |
| yfiH | 37 | SEQ ID NO: 82/ | Laccase | 47.06 | 85.71 | 0.082 |
| | | SEQ ID NO: 127 | domain | | | |
| | | | protein | | | |
| group_ | 38 | SEQ ID NO: 83/ | hypothetical | 47.06 | 85.71 | 0.082 |
| 8391 | | SEQ ID NO: 128 | protein | | | |
| cyaA_ | 39 | SEQ ID NO: 84/ | Adenylate | 47.06 | 85.71 | 0.082 |
| 4 | | SEQ ID NO: 129 | cyclase 1 | | | |
| Vgb_3 | 40 | SEQ ID NO: 85/ | Virginia- | 47.06 | 85.71 | 0.082 |
| | | SEQ ID NO: 130 | mycin | | | |
| | | | B lyase | | | |
| Group_ | 41 | SEQ ID NO: 86/ | hypothetical | 47.06 | 85.71 | 0.082 |
| 52947 | | SEQ ID NO: 131 | protein | | | |
| pimB_2 | 42 | SEQ ID NO: 87/ | GDP- | 47.06 | 95.24 | 0.084 |
| | | SEQ ID NO: 132 | mannose- | | | |
| | | | dependent | | | |
| | | | alpha-(1-6)- | | | |
| | | | phospha- | | | |
| | | | tidylinositol | | | |
| | | | monoman- | | | |
| | | | noside | | | |
| | | | mannosyl- | | | |
| | | | transferase | | | |
| bmr3_2 | 43 | SEQ ID NO: 88/ | Multidrug | 29.41 | 100.00 | 0.086 |
| | | SEQ ID NO: 133 | resistance | | | |
| | | | protein 3 | | | |
| fabD_1 | 44 | SEQ ID NO: 89/ | Malonyl | 47.06 | 85.71 | 0.093 |
| | | SEQ ID NO: 134 | CoA-acyl | | | |
| | | | carrier | | | |
| | | | protein | | | |
| | | | transacylase | | | |
| group_ | 45 | SEQ ID NO: 90/ | hypothetical | 35.29 | 100.00 | 0.097 |
| 12146 | | SEQ ID NO: 135 | protein | | | |

Sequences of consensus proteins SEQ ID NO:28 through SEQ ID NO:45 are provided below. In the consensus sequences, X can be any amino acid residue or can be absent.

SEQ ID NO: 28
XXXXXXXXXXXXXXXLXDXXXGRVYVDCXSCKXXXXXXXRXGRYXVXSLXERF
GXDISTLXXDLLRXLTASCRYQRXPGAPPARKYEHLCLAAITLPXXXKXXXPVPPGV
PYTIEVWXEXGGXIELHLAXIYPLXMAXAAFEXACXXWPXHEVTLRDRXRIVXKRE
RPXXXATXXXXXXXXXXXXXXXXXXXXXXXXXXXXXXXXXXXXXXXXXXXXX
XXXXXXXXXXXXXXXXXXXXXXXXXXXXX
SEQ ID NO: 29
XXXXXXXXXXXXXXXXXVIHDXKXPVTRXLVLVRHGQSVANRSGLFTGLLDSPLT
EQGRXEAVAAGRRLAERSWRFSDAFTSTLTRAVVSGRLILDTLGQPGLIPQRFAALD
ERDYGDLSGLDKTAADARWGAERIETWRRSYAEAPPNGESLRDTVARIVPCYLRSIL
PAVMGGDVLVVAHGNCLRALVMALDDLSPAEVEXLELATGSVRIYEFAADTTIEAR
WIDG
SEQ ID NO: 30
LDEIAPLIEPQIPALRRYAVALLRDREAADDLVQDTLERALSAWSGRRRDGDLRAWL
FTIERNLFLAAVRRRGRRGADLGAEALEQVPDPSADPGAALGARDVLAGLDTLPEEQ
RSVLLLVAVEDLSYAEAAQVLGVPLGTVMSRLSRARTRMRGFVETGRTGLLRRVK
SEQ ID NO: 31
MATSPIWHVVSALAVAGTLAVHGGPAHAAPRQATAGALHFDDGGESYRGYRIEMA
QDVPNAEIGQLRQAAEHQVDIVEATSLDEGTKAFLRRFPVVVXSGSGEXSHYSGGDX
VTIAVEDPKDDRPILLHEYMHVYHFRKMPGGRNNPDILTFYGRAKDGGFYPAGAYL
LKNQGEFFAMTASVYLHGRLAREPFTRDELRQKQPVYYRFLTRLFGPVGA
SEQ ID NO: 32
MTVRNIAACVLLLCTGPALADTLPLRHGAYVSVGTDCKDPPNVALRTYDGGGLGSS
KANDCRSRVLSKQGNVFEIEQDCRQFGGPKVERATERSTIRVDGPDRYTDLTXGXGE
SFRLCPGLKP
SEQ ID NO: 33
XXXXXXXXXXXXXXXXXXXXXXXXXXXXXXXXXXXXXXXXXXXXX
XXXXXXXXXXXXXXXXXXXXXXXXXXXXXXXXXXXXXXXXXXXXX
XXXXXXXXXXXXXXXXXXXXXXXXXXXXXXXXXXXXXXXXXXXXX
XXXXXXXXXXXXXXXXXXXXXXXXXXXXXXXXXXXXXXXXXXXXX
XXXXXXXXXXXXXXXXXXXXXXXXXXXXXXXXXXXXXXXXXXXXX
XXXXXXXXXXXXXXXXXXXXXXXXXXXXXXXXXXXXXXXXXXXXX
XXXXXXXXXXXXXXXXXXXXXXXXXXXXXXXXXXXXXXXXXXXXX
XXXXXXXXXXXXXXXXXXXXXXXXXXXXXXXXXXXXXXXXXXXX
MALQXXADXXAAXLDAXEQXAAXLLDGXXQGLGALGEAAGQGFAXGVHQGAEX
XGXALEXXXXXLGAXVDQXGERXLAXXERXGQXRXVGLDLXXMGXRPXVEGGX
QXXXALVHEAXEGLVAVVEGXGQGVGALXXQXREXXXAAVDXLGEGIXAGVDXA
GQXLXXXXXXXXXXXXAALXDXVEVXQXGVEXLXQGXXXXXXXXXXXXXXXX
XXXXXXXXXXXXXXXXXXXXXXXXXXXXXXXXXXXXXXXXXXXXXXXXXXAX
XXLGXXXQXXAAXLQEPLDXRAXLLEGXXQGXXXXXXXXXXXXXXXXXXXXXX
XXXXXXXXXXXXXXXXXXXXXXXXXXXXXXXXXXXXXXXXXXXXXXXXXXXX
XXXXXXXXXXXXXXXXXXXXXXXXXXXXXXXXXXXXXXXXXXXXXXXXXXXX
XXXXXXXXXXXXXXXXXXXXXXXXXXXXXXXXXXXXXXXXXXXXXXXXXXXX
XXXXXXXXXXXXXXXXXXXXXXXXXXXXXXXREAGVQQVXHXGGRRVEXXAX
XLGXGXDXVGDVXAXXVHEVAQAXLGLLEQGXQALXAXVEGXGELALAGLDQXR
XGGGXLLQXXAQGLXAXVXXGGEAAAAAXDXXVEGXLGLLEQXAQXLGXGXEG
VGEAVAXAVDQXLQXAEXXVEQXAEXXGAXVQXAGGXXERAXQXXDEXXXXAV
QXLXDXAXAXVQGXXQGAGVXVQHDADLVGXVVQQXVXXAHALVEQAADVXX
AARQXVAQAALAXIEHPLGVXDGLLELGEXALAXLVDEXGERGAAXVEQAGELGD
XXADXXEHXXALLVELXRHXXXALVDEAXEQVGAXGEGGXHXLGAGXQXXVGX
XGALVHQXXXXLRAXRQXGLEXAGXLLDGAGEGXDALGHGXVEAAGXXXDXXH
EVAGXXGQLXVXXAGARLDQADRLEGLXAEALVHEAGALVQXXXDXLQAAXDX
XVEXXGAXLDXAGDLAXXAXDVXAEXXGAXXXXXXXXXXXXXXXXXXXXXXX
LXXLHQAAXAXXXDPGHLXXXGXQXLVQGXGAXXXXXXXXXXXXXXXXXXXX
XXXXXXXXXXXXXXXXXXXXXXXXXXXXXXXXXXXXXXXXXXXXXXXADMX
VDLADQXGHGXGXXGGXLXXXXXXXGXAXLQXAEMGLDQAAGLAGXXRDXLGH
GLAXXGEEXXXLLQXXAXGLAEGGGVAADPVGQDVAXGDGLLOGGEAAAQXLVD
XXDPXVGGLGEXXAXXAEXVGDAXALARHGEHDVAAGAXDXGLGLVXVAGEQL
GXXAAMXAEGXXXLADXXXEVGRDXXGGXXEXAGXXAGXGGEFXVEXXEGXAX
XRDGGXAXXAEXXXXXVGGVAEXGXEDXGXXLDGXXDLXXGGAQXXQXXGEXX
XGIXXXXLGXLAXLXGGXAXXXGXXXXXXXXXXXXXXXXXXXXXXXXXXXXXX
XXXXXXXXXXXXXXXXXXXXXXXXXXXXXXXXXXXXXLXGAAVDLARQXLDX
XXQXXXXXXGXXLEAXAQGREGXLQAXXXXXXPXXDXAAGXLGRRXDXVLQXR
XXDXXGLVEAXXDAVHPLGEXAAAXXXGIXDPLGGXDDVVAHXXGAGXQXXXX
AXXRXXQGXLXXXXAXXDXXXDXXAXXLDLGAQXXAXAXXVLXEGRAXLGEAX
GDLGAXXXEALXXXXXXXXXXXGQXXAPXQEGXXQXRXGALEALXHRXXLXXX
XXADAXXGLXQGXXDXAXXLGQXXGQGXXXXLEXAGXXXGAILEXAXQXXADX
XEAXAHXXAGXRQXVDQXXAAXXHQXXXXVAXLAQXGGDXXAXXLQGAGHAL
AXXXXGRDDPVGXXIQXLXQXXVRAGDGAAHPLGVGDDXLALXXQLVXEGADAD
LVVGIGXLQGGDLAAHQGXQLAXPGEXXLDAVAHRRXLAAXGLRHGQXXVXXEA
LXLGQAHRDLAHXAGDEAHLLGAHXQHXXDXEQXHRXEQRRXXXXXLEXXEAXX
DXXQVAXXLRPXDQXEXXDPXXAGXXGDXVGLXRGAHPQGLLQDADVAPVVVG
HQRAVGRQEAALXAGXVQXXXXQVXGEXRVXXXXXXXXXXXXDVEAXRXXXXG
GVXXAVEQRIGVXDIXQXXLXXXXXAVXXQGVEXQXLLDXXXXXXXXXXXXXXX
XXXXXXXXXXXXXXXXXXXXXXXXXXXXXXXXXXXXXXXXXXXXXXXXXXXX
XXXXXXXXXXXXXXXXXXXXXXXXXXXXXXXXXXXXXXXXXXXXXXXXXXXX
XXXXXXXXXXXXXXXXXXXXXXXXXXXXXXXXXXXXXXXXXXXXXXXXXXXX
XXXXXXXXXXXXXXXXXXXXXXXXXXXXXXXXXXXXXXXXXXXXXX
SEQ ID NO: 34
MLAMNYRGPYRVRVDRKPVPVCEHVEDAVIRVTRTCICGSDLHLYHGMVPDTRVG
MTFGHEFTGVVEEVGSGVRNLAVGDHVLVPFNIACGRCHFCKQGLFGNCHEANAQ
ATAVGGIYGYSHTAGGYDGGQAEYVRVPYADVGPCKIPXTMDLDDAVLLTDVVPT
GYQAAEMGGIQRGDTVVVFGAGPVGIMAARCAWLFGAGRVIVIDHVEYRLDFARR
YCPAEVYNFRSIGDPVVFLKKTTDSLGADVCIDAVGGDAAGSALHTLMGTKLKLEG
GSAXALHWAINSVKKGGIVSIVGVYGPTGNLIPIGNVLNKGITIRANQASVKRLLPKLI
XHIEAGRLNPKELITHKVPLEEAADAYHLFSAKLDGCIKPVLVPPTARXXXXXX
SEQ ID NO: 35
XXXXMRNRPIDPSTVPGWGVDADPQNDPTYPMRDXARDDXXXXXXXXXXRGMN
WXRPPXXXXQQRARVEVLTSIEYNXRPAVXGTSTPPRGVSGVIRRQAFRYSESQWA
HWLMLMAADRVNVVEGVVDDLXRGRVPNVPAEMGARAELAHNRSGLAXKLALT
GAAIGLGILVSRLARAQRRXXXXXXXXXXXXXXXXXXXXXXXXXXXXXXXX
SEQ ID NO: 36
XXXXXXXXXXXXXXMPPLLALPFPAIDPVAVSIGPIEIKWYALAYIAGLVGGWYYA
RRLVXADSLWGVVKRPTLXDIDDLIVWVALGVVLGGRIGYVLFYNLPMYLXHPXEI
LAIRNGGMSFHGGFLGAILAXLLFARSRGLNGYTMLDIXAVVVPIGLFFGRIANFVNG
ELWGRAAPDFPYAVVFPTGGPVPRHPSQLYEAATEGLLLFIVMALAVRRFGFRKPGL
LGGIFVLGYALARTFCEFFREPDRQLGFLFGXDVXXMGGGVTMGMLLCVPMXLVG
LXYIVLAARGXTRPRKTXXEIXAXEAXQXXXXXX
SEQ ID NO: 37
XXXXXXXXXXXXXMFIEAPELSSHSXIRHAFFTRQGGVSEGLYASLNGGLGSNDDPE
RVAENRARMCAQLGLPRDXNLVSLYQVHSAEVVTVEAPFPXAERPKADAMVTRVP
GLALGIATADCGPILFADPENRVVGAAHAGWKGALXGVIEATVAAMEALGAERRSI
VAVLGPTIAQASYEVGPDFVERFRAEXPGXERFFXXGXXRPGHAQFDLPGFILXRLA
EAGIGEATALGLCTYADPERFYSFRRTTHRNEPDYGRLISAIALTPXXXX
SEQ ID NO: 38
XXXXATPLXAELARXIRXSGPIGIDRYMALCLGHPVHGYYRTRDPXGXXGDFTTAPE
ISQMFGELVGAWAAXVXXXMGAXXXPDXXXLVELGPGRGTLMADALRALRAAXX
GXXAXXEXHLVETSPVLRRLQAXRLAXAXXXXXXXXXXXXXXXXPTWHDSVDTL
PDGPLIIIANEFFDXLPVRQFVRTXXGWCERRVGLXXXXXPEGXALAFGLXPEPXPXL
XAXXXXXEAPXGAVLTLPSXGLALMRXLARRLXARGGALLAIDYGHXRPGFGDTX
QAVAGHRFADPLAXPGEADLTXHVDFXALARAAAAEGAAIHGPVTQRDFLLXLGL
AXRAERLKARATPDQAQAIDAAVXRLTDPXPRGMGALFKVLXVSXPALGPLPXLPP
XXXXXXXPXXXXXXXXXXXXXXXXXXX
SEQ ID NO: 39
XXXXXXXXXXXXXXXXXXXMGPGREAVDAAALLDGFSACLSGLGLPLARATTHA
PTLHPSFRWVMRVWHPGTSSLALRRRHGIEGTPTFHGNTVEHVVETRTPLQCRLDGD
GPLPFPVLGELRNEGLTDYLIAPLXAARGRMGAASWATARPGGFTPIEIETLLALVEP
FSLLFEIKALDDMLGAVLSAYVGRDPARQILAGTVRRGDVRLMRAAMMLTDLRGFG
ELSDRQSPDHVVAALNRMFDAIVPAVEAEGGEVLKYIGDGLLAVFDADRDEAEARR
AALRAAEAALDALATLRDGDRAAFEVGVALHVGEVAYGNIGGGDRVDFTAIGRDL
NVLARVERLCKTYDTPLIATDTFLHGLAHALEPLGIVALRGFAERHALFGCRRTAPV
EAPAVXX
SEQ ID NO: 40
MTHIRTSLRAXLAGAALLLAQGQPGSAAGFDGAIKNNALALNAAGTVAAVSNSEES
AVVVYDVAKGTVLRRLDGFVTPRNIVFAPDGARFYVSDSGTGRITVYETATGKEAG
VLAAGPGAFGTVLSADGGKLYVNNEAASTLTVFDTKTMLADAVIPGFAQPRQGVKL
SPDGKTVFVTNFLGDKITLVDTATNKITGEIAGFDKLRAISITKDGKTLFAANSGRNTI
GVVDVAARKVTSEVEVGKDPYGAALTPDGRFVYSGNLKDNSLSVIDTGTLKVVATV
TGLNEPRQAIAFSADNARAYVLNRDLSVAVVDRAKNAVVSTMKP
SEQ ID NO: 41
MSEPAKPVPPDDPRVRLAEDRTVLAAERTFVAWLRTGLAFLGVGLAAQRFLREVLA
VWPLKVLSLTLIXCALASFAGAAWRDRAIRARLAHAEIPMMPRILTVGIAALLIAISG
LAATALLWA
SEQ ID NO: 42
XXXXXXXXXXXXXXXXXXXXXXXXXXXXXXXXXXXXXXXXXXXXXXMRVAIV
HYWLIGMRGGEKVVEALCDLYPEADIFTHAYAPQSMSPTIRAHRVRTSFIGRLPFATS
RYKSYLPLMPMALEQLDLRGYDLIISSESGPAKGIIPPSDALHICYCHSPMRYVWNMY
HDYRERTGLLTRLLMPPVAHYVRNWDAVSAGRVHEFIANSDTVARRIETYYRRQAK
VIHPPVDTAAFEXXPDGERGDYHLMVGEMVRYKRPELAIQAFNRLGQPLVVIGGGE
MLRELRSMAGPHIKILGPQPFEVLKHHYARCQALIFPGEEDFGIVPVEAMASGRPVVA
FGKGGVTETVIDGVTGTFFHEQSVDALIDAVQRCRAIGVEPERLVRRAADFGVGRFA
DEISRFVDGVLARERLAAPRPXXXXXXXXXXXXXXXXXXXXXXXXXXPREPSRAY
LVQXXXX
SEQ ID NO: 43
VAXXXXXXXSPXAVTXTQXQAQARPXAPQXRLNPXEIRAIVYGLMTAMLLAALDQ
TIVATAMPTIGLDLGDAANLPWIVTAYLLASTAVTPLYGKLSDIHGRRIMLLIAIATFV
LGSLACALAPTMVTLALARGLQGIGGGGLIALSQTILADIMSPKERARYQVVIAGVFV
AASVAGPLLGGLFAQHLHWSLIFWINLPIGILAFALTNANLKRLPRHERRHRLDWPG
AALMVAGSVTLLLALSWGGVRYPWGSAPVLALLAGAAVLGGGFAARLATAAEPLI
PTEVLKDRVVYSATLAACFAMGTFIGLTIYVPIFLEGVIGLSASESGVALVPLMIGTVT
GATLSGRSMLHFRHYKRVPLAMMXLSLACCATIAWEGRALPFWLMEVLFALLSMGI
GTILPLSTIAIQNAVEPHQLGIATAAMNFFRSLGGALIVAAFGTIVLGGAAGGAGXXX
GGHDVESLIRGADPAQLALTFRXVFLAACLGLLGAFTFLALMEERPLRERXSPRMGG
EPXXGXPX
SEQ ID NO: 44
MTLAILLSGQGGQHPXMFDLTADHPAAQXVFSAARPLLGGTDXXXXPRDLARAXX
XXXXXXXXGGDXLHXNRTGQILCCVAALAAWRALXXXXXGRAIVAGYSIGDLAA
WGVAGRXDXADILALAARRAEAMDAAXXXXXSGEGFGLAGIRGLTLDALDDLAXR
HGCHLAIRNAADSGVVGGRRDALDALCRDAXAAGAQRAVXLXVHTPSHTPLLAAA
SEAFRDALAXVXLRRPPXGAPRLLSGLDGTTVFRPEDGLDKLARQISHTIDWAACLE
ACREYGADRVLELGPGHALATMARXALPXARVHALEEFRSVDGVADWLXRPXXX
XXXX
SEQ ID NO: 45
XXXXMSGPAPDXGRLTVIGVRHHSPACAGLVRRTIAALRPAFVLIEGPVDFNPHLPD
LALGHDLPVAIFSFRADAXGSAASYTPFCAFSPEWQALEAGRAVGAQTLFCDLPAW
DPAFGRRANRYADPHGARAEAAERALAAALGVADQDALWDVLAEAAPEAELPARL
DRYFALLRPPGTDDPAEEARERFMGAYAAHALRAAGDRPVVLVCGGWHADAVRR
HAAQADGTRPEPAPPEPDLRTGSYVVPYAYPRLDRESGYAAGMPAPGYYERVAEAG
LAPAADWAMTAITAALREAGQVVSTADRIAWRVHAEALARLRAHPAILRADLIDAA
LAALVKDALDRPPAWAAGGAAPGHPALAAMLRALTGRREGRLAPGTRQPPLVADV
AERLRAADLEPGPARRSIDLDWAEPGDRARAHLLHRLXLLGLPGIARXEGPDRAEPG
LPRERFTLVRHPHWLGALIEASLWGGTLEMAAAARITARVEAAPDSLAVLTGALSD
ALFAGLTLEGDLLARLSAGIAAAHDXAALGAAGAGIVRLYRFGDAFXAPARPALAR
LCAALAARALFVVEGIRDPRAGLGAIPLLLACRDLFREVGAEVXGLDELRXPFAAML
GRRLADPETPPALAGAALGFRVACGAAGSDPEAALSRLRRFGLPATLGDFLAGLFAL
AREEIAADATLASVEGLVAAWGDDDFLRALPSLRMAFAWFPPRERERIAVAILRRSG
LGEARAEVEALAWMRQRARPADQAEALAREARVAARLARYGLT

### Example 6 Ag Chemistry Tolerance Screen

Methylobacterium strains were screened for tolerance to commonly used agricultural chemicals using a plate assay as follows. Agar plates containing AMS-GluPP media plus one of the below listed chemicals were prepared with concentrations calculated to approximate the amount that each seed would be exposed to in the field at the middle recommended treatment rate. Concentrations of the chemicals in the plates are provided in Table 4 below.

TABLE 4

| | Concen- | Treatment used | Chemical Rate in |
|---|---|---|---|
| Chemistry | tration | in Field | Field (Mid-rate) |
| ILeVO (Fluopyram) | 3.596 mL/L | Seed Treatment | 1.0 fl oz/100 lbs seed |
| Axyl Shield (Metalaxyl) | 0.986 mL/L | Seed Treatment | 1.58 fl oz/140,000 seeds |
| Xtendimax (Dicamba) | 160.8 uL/L | Foliar Spray | 22 fl oz/Acre* |
| Headline (Pyraclostrobin) | 65.8 uL/L | Foliar Spray | 9 fl oz/Acre* |

*used 10X rate

Bacterial strains to be tested were grown for 3-5 days at either 25 or 30° C. in 96 well plates in AMS-GluPP media. Using a p200 multichannel pipette set to 175 uL, cultures were pipetted up and down approximately 10 times to ensure uniform turbidity throughout. Plates were spotted carefully (to avoid puncturing agar) using a p20 multichannel pipette set to 3.2 uL and dispensed until the first stop only to prevent excess spray spots on the plates. Three replicate plates were spotted for each of the strains to be tested. Plates were allowed to fully dry and then inverted and incubated for 5-7 days at room temperature or 30° C. Following incubation, plates were scanned using an Epson scanner. Growth of Methylobacterium on plates was visually scored using a rating of 0-3, 0 representing no growth and 3 representing full growth. Control plates were used for comparison to the AgChem plates to ensure accuracy. Scores for each of the three reps were averaged. Strains with a score of greater than or equal to 1.66 were identified as tolerant for a given ag chemical. Strains with a score of ≥1.66 for each of the ag chemicals tested were considered tolerant to agricultural chemicals.

Having illustrated and described various embodiments, it should be apparent to persons skilled in the art that the disclosure can be modified in arrangement and detail without departing from such principles.

Although the materials and methods of this disclosure have been described in terms of various embodiments and illustrative examples, it will be apparent to those of skill in the art that variations can be applied to the materials and methods described herein without departing from the concept, spirit and scope of the disclosure. All such similar substitutes and modifications apparent to those skilled in the art are deemed to be within the spirit, scope and concept of the disclosure as defined by the appended claims or otherwise disclosed herein.

What is claimed is:

1. A method for identifying one or more genetic elements correlated with colonization efficiency of a plant-associated microorganism, wherein said method comprises: (i) screening a population of plant-associated microorganisms to determine the ability of strains in said population to colonize a plant or plant part; (ii) identifying a first set of strains in said population that colonize said plant or plant part at an enhanced density as compared to a non-colonizing control treatment or other strains of said population; (iii) identifying a second set of strains in said population that colonize said plant or plant part at a reduced density as compared to other strains of said population, or at a density that is reduced or not significantly different from that of a non-colonizing control treatment; (iv) comparing the sequences of genetic elements in said first set of strains and said second set of strains; and (v) identifying one or more genetic elements that correlate with colonization efficiency.

2. (canceled)

3. The method of claim 1, wherein (i) said one or more genetic elements that are positively correlated with colonization efficiency comprise at least one gene selected from the group consisting of bbsG, hdhA, luxQ, bicA, hddC, hddA, fptA, livF, sutR, cdhR, amaB, ssuA, rbn, ftsY, fecA, gpmA_2, ecfG_1, adh, lgt, yfih, cyaA, vgb_3, pimB_2, bmr3_2, and fabD_1; or (ii) said one or more genetic elements that are positively correlated with colonization efficiency encode at least one protein having an amino acid sequence selected from the group consisting of SEQ ID NO: 1 to SEQ ID NO: 44, and SEQ ID NO: 45 and/or encode at least one protein having at least 50% sequence identity to a protein selected from the group consisting of SEQ ID NO: 91 to SEQ ID NO: 134; and SEQ ID NO: 135.

4. The method of claim 1 wherein said plant-associated microorganisms are pink-pigmented facultative methylotrophic (PPFM) bacteria.

5-6. (canceled)

7. The method of claim 1 wherein said plant part is a stem, leaf, root, tuber, or seed.

8-9. (canceled)

10. The method of claim 1 wherein said plant is a soybean plant or a corn plant.

11-12. (canceled)

13. The method of claim 4 wherein said population comprises 10 or more strains of pink-pigmented facultative methylotrophic (PPFM) bacteria.

14. The method of claim 1 wherein said plant-associated microorganisms in said population are desiccation tolerant and/or tolerant to the presence of one or more agricultural chemicals.

15. (canceled)

16. The method of claim 14 wherein said microorganisms are tolerant to one or more agricultural chemicals is selected from fluopyram, metalaxyl, dicamba, and pyraclostrobin.

17. A method of selecting a microbial strain capable of efficiently colonizing a plant, plant cell or plant part, wherein said method comprises detecting in the genome of said microbial strain one or more genetic elements that are positively correlated with colonization efficiency.

18. (canceled)

19. The method of claim 17 wherein (i) said one or more genetic elements that are positively correlated with colonization efficiency comprise at least one gene selected from the group consisting of bbsG, hdhA, luxQ, bicA, hddC, hddA, fptA, livF, sutR, cdhR, amaB, ssuA, rbn, ftsY, fecA, gpmA_2, ecfG_1, adh, lgt, yfih, cyaA, vgb_3, pimB_2, bmr3_2, and fabD_1; or (ii) wherein said one or more genetic elements that are positively correlated with colonization efficiency encode at least one protein having an amino acid sequence selected from the group consisting of SEQ ID NO: 1 to SEQ ID NO: 44, and 45 or encode at least one protein having at least 50% sequence identity to a protein selected from the group consisting of SEQ ID NO: 91 to SEQ ID NO:134, and SEQ ID NO: 135.

20. The method of claim 17 wherein said microbial strain is a pink-pigmented facultative methylotrophic (PPFM) bacterium.

21. (canceled)

22. The method of claim 17 wherein said plant part is a stem, leaf, root, tuber, or seed.

23-24. (canceled)

25. The method of claim 17 wherein said plant is a soybean plant or a corn plant.

26. (canceled)

27. The method of claim 17 wherein said microbial strain is an endophyte.

28-30. (canceled)

31. A method of selecting a microbial strain capable of efficiently colonizing a plant, plant cell, or plant part, wherein said method comprises detecting the presence of one or more genetic elements in the genome of said microbial strain, wherein said one or more genetic elements (i) comprise a gene selected from the group consisting of bbsG, hdhA, luxQ, bicA, hddC, hddA, fptA, livF, sutR, cdhR, amaB, ssuA, rbn, ftsY, fecA, gpmA_2, ecfG_1, adh, lgt, yfih, cyaA, vgb_3, pimB_2, bmr3_2, and fabD_1, or (ii) encode at least one protein having an amino acid sequence selected from the group consisting of SEQ ID NO: 1 to SEQ ID NO: 44, and SEQ ID NO: 45; or (iii) encode at least one protein having at least 50% sequence identity to a protein selected from the group consisting of SEQ ID NO: 91 to SEQ ID NO: 134, and SEQ ID NO: 135.

32. (canceled)

33. The method of claim 31 wherein said one or more genetic elements encode a protein having an amino acid sequence with at least 80% identity over the entire region of a sequence selected from SEQ ID NO: 91 to SEQ ID NO:135.

34. The method of claim 31 wherein said one or more genetic elements encode a protein having an amino acid sequence with at least 90% identity over the entire region of a sequence selected from SEQ ID NO: 91 to SEQ ID NO:135.

35. The method of claim 31 wherein said method comprises detecting the presence of two or more genetic elements selected from the group consisting of genetic elements that encode a protein having at least 50, 60%, 70%, 75%, 80%, 85%, 90%, or 95% percent identity over the entire region any one of SEQ ID NO: 91 to SEQ ID NO:135.

36. (canceled)

37. A method of identifying a yield-enhancing plant-associated microorganism, wherein said method comprises: (i) screening a population of plant-associated microorganisms to identify strains having tolerance to desiccation and tolerance to contact with agricultural chemicals, (ii) screening said population of plant-associated microorganisms to determine the ability of strains in said population to colonize a target plant or target plant part; (iii) identifying strains in said population that are tolerant to desiccation, tolerant to contact with agricultural chemicals, and that colonize said target plant or target plant part at an enhanced density as compared to a non-colonizing control treatment; (iv) contacting strains identified in (iii) with said target plant host or a part of said target plant host; (v) growing said plant host under field conditions; (vi) determining harvested yield of said plant host; and (vii) identifying strains that improve yield of said plant host.

38. The method of claim 37 wherein said yield-enhancing plant-associated microorganism is a Methylobacterium.

39-40. (canceled)

41. The method of claim 37 wherein said plant host is a soybean plant.

42. (canceled)

43. The method of claim 37 wherein said screening in (ii) comprises comparing strains in a colonization screen to identify strains that colonize said plant or plant part at an enhanced density.

44. The method of claim 37 wherein said screening in (ii) comprises detecting in the genome of said microbial strain one or more genetic elements that are positively correlated with colonization efficiency.

45-46. (canceled)


SEQUENCE LISTING (135 sequences)

SEQ ID NO: 1 - protein, 563 aa, synthetic construct
XXXXXXXXXXXXXXXXXXXXXXXXXXXXXXXXXXXXXXXMXXXXXXXXXXXXXXXXXXXXXXXXXXXRRRXGXXMLLGTLAAGXXXXRPAXAEPVVEDVRIVDFDWVDXARQRXVPARLYWPNTSSLRRXVPLVVFSHGLGQSRTGYSYLGRHWSSHGIASLHLQHVGSDSTVWTGNPLALLDRIDXAAXEREAIARARDLRFXLDRLLVQDGGXFGDRIDXRRIVAAGHSYGANTTLIAAGARVIRDGXPLQXRDPRXXAGIVISAPPFYGERDLRAVLXAVDIPTLHVTATEDXIQLPGRTXSPXXDRLXVYEAIXTPRKALAVFQGGSXXXXXXXXXXXXXXXHSIFTDXXXXXRPLTGGXNLNPQVKXATAXGXLAFLDLAFRGDPEPLXXXXXXXXXXXXRAWSSTWXXXXXXXXXXXXXXXXXXXXXXXXXXXXXXXXXXXXXXXXXXXXXXXXXXXXXXXXXXXXXXXXKPILAVAPAGFXXXSXAQPAVAXRXRDRXXXXXXEPLXXXXXXXXXXXXXXXXXXXXXXXXXXXXXXXXXXXXXXXXXXXXXXXXXX

SEQ ID NO: 2 - protein, 510 aa, synthetic construct
XXXXXXXXXXXXXXXXXXXXXXXXXXXXXXXXXXXXXXXXXXXXXXXXMRKKAXNRXXXXXXXXFTAXXXXXXXILXVAAXXXXGTXXXXLXLGAAEAXRDQXGNYAQAEWAQDYASPAGMQVQRETXPILSPQTVAATEQMVERYRDIVNRGGWRPVSGAERLRVGSKGPAVAAVRQRLIVTGDLDPAAGGSGVYDSYVAAGVKRFQARHGLSQTGAMSMATQQAMNVPADVRLXQLETNVVRLRSYSGDLGRRFVITNIPAALVETVENGQVVTLHAAGVGKIDRQSPIMNTKATQINFNPTWTVPASIVKKDLIPKMQKDPNYLTDNKIRILSGXXEISPRSVNWNSDEGTRYTYRQDSGADFNSMGIVRINIPNPYGVFMHDTNTKGVFGDDFRFISSGCVRVQNVREYITWLLKDTPGWXREQVEQAIESGKRVDANIAQPVPVYWTYITAWATPDGLVQFRDDIYKRDGVNVPSTIGAPTPVASAEPLXPQTFEPGDXEEXXXX

SEQ ID NO: 3 - protein, 462 aa, synthetic construct
  REGION 1..462: note = Consensus sequence
XXXXXXXXXXXXXXXXXXXXXXXXXXXXXXXXXXXXXXXXXXXXXXXXXXXMTEIDTDAFLADLYALREIGRFRTGVHRPTFSAXDMESRRWLMAKLEXCGLEASIDGIGNVLGRHRGPGPHLLVGSHIETQNEAGWLDGALGVVAGLALARAGLPVDVVAFADEEGHFSGGFLGSRSAIGDLTEAEIDAARNRTDGTPLRAALESAGLAGLPRMRLDPARYRGFLELHIEQGTQLESAGLHLGVVSGIVAIWQFQIVFDGNQDHAGGTTMAERRDAGLSAVRLLAAIDREFPKVCGPRSTWTTGRITLDPGGYSIIPGRAEVAFQFRDVSMPVLERMEACLEALVRESNRRERCPATLTALSKAIPAPCDPDLMRALSEAAEQVCPGRWQVMPSGAGHDAQNIARILPAAMLFVPSIGGISHHWAEDTSDADLAFGVRALGAAAARVLAGXXXXXXXXXXX

SEQ ID NO: 4 - protein, 220 aa, synthetic construct
  REGION 1..220: note = Consensus sequence
XXXMRRPDQPALTAAAHGRVSMPFLPTLAVVAILGVASLYCPTSKPGQPXGARVEXXXXXXXXXXXXXXAGVSAAPGPTDFAPIAAXVAAPGRPPAVIAFAEQYPLDAXVIARTGSLPARPAVAARANAHVAAAGRRACPGRRCPETPRSNTDPMAPARGXAADEAEDALLPSQAMPFAASVVETLVPAARAVGDAANLVRSSARAVQGTVALAVADCLR

SEQ ID NO: 5 - protein, 313 aa, synthetic construct
MSNSSEPTILAADQDLALRFWGVRGSTPVSGPQYAEFGGSTPCIEVRCGQRMFIVDAGSGIYNLGQGHRTDLPQEVDLLFSHLHLDHTAGLPFFKPAVLDPDRVINTYCGNLGGESAGPTLDRLFAPPLFPVTLDKLCCTFHHHGFEAGQTLAFPDGTRVATILLNHPQGSVGYRFEHGGRRLCLISDIEHSDPWPDPELAAFVADADLMVYDGMFTDGEYPTCRGWGHSTWQKGVELARNAGVKALGIIHLHPAHSDTALRDMEADLQAEMPTAFIARERQSLIVGAPRAVSGRPSGRPAVAREMRRRIKVA

SEQ ID NO: 6 - protein, 625 aa, synthetic construct
XXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXMXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXSVXIXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXLXXXXPXXXMRIVXXXXXXAAXXXXXXXXXXXXXXXXXXXXXXXXXXXXXXXAAXXXXXAPXXXXXXAAXXVKAGDLXIETPWLRATPGGAKVAGGYVRIXNTGSXPDRLTGASIPXAGRGEIHSMSMEGGVMKMAPVXGGLXIKPGETVELKPGGYHLMFEDLTXAPKAGETVSGTLTFERAGTVPVTFTVAPIGAXAXPXXXXXXXXXXXXXXXXXXXXXXXXXXXGHQHXHHXXXX

SEQ ID NO: 7 - protein, 67 aa, synthetic construct
MXXXXXXXXXRSAGDAXSASLMDQIAPARTGRTRRRPHPALVVASVALAIGLWFVIYAVAGRAIWFW

SEQ ID NO: 8 - protein, 439 aa, synthetic construct
XXXXXXXXXXXXXXXXXXXXXXVDFTLSXSQTHWLTRVRAFIADAILPAAAAVAAERAQSRXPSPTMERLKDKARXEGLWNLFLPPSPEHDTDEYRGAGLTNLDYALCAEEMGRVGIASEVFNCAAPDTGNMEVLHRYGTXAQKDRWLKPLMAGEIRSAFLMTEPEVASSDATNISTSIRRDGDHYVIDGRKWWSTGVGDPRCRIAILMGKTDPEAPRHRQQSQILVPMDTPGVRVERLLPVFGYEDAPKGHGEVVLENVRVPAGNLILGEGRGFEIAQGRLGPGRIHHCMRTIGAAEVALEAMARRLVSRVAFGKRISEQSVWEQRVAEARIDIEMTRLLCLKAADMMDKVGNKGAKLEIAMIKVAAPRVALKVIDDAIQAHGGAGVSEDFGLARMYAHIRTLRLADGPDEVHNRSIARLEFGRYTNARXGXXXXXXX

SEQ ID NO: 9 - protein, 821 aa, synthetic construct
XXXXXXXXXXXXXXXXMPRXFRXGXXXRAALLXXXXLXXXATPAAAQXXXXXXXSAASVTLXELSVXGAAAXXXXXXPXXXEXXGSLTVPSVXRQRAALNXTVGSVAFVDAXXXQDRYANTLRDVLKDVPGVYVQERYGQELRLSVRGSGIARGFHLRGLELLQDGIPLNLADGSGDFYQVDPLALRSVEVYKGGNALTFGATTLGGAVNXVTPTAYTALAPNILRVDGGSFGTIRENFQMSRIXGPLDXLVNGTLTNSDGFRXHEAQRTQNFNANIGYRIAPGIETRFYLGXYLTDQKLPGTLTLGQSLSTPTLANPTAITGNQSRKVETERIANRTSFLLDVGKLDIDTWAIHKSLYHPIFQVIDQDGWTYGISPHWAGTFDIGGFRNDTILGLRAFAGQNSALQFVNVRGQRGAQTLNALQSASNXEAYGENRFWFLPDVALMTGAKAFSSNRTYSDKGGXPXGNPXXRFADVTYEGVNPKIGLLWQPXPDIQVFGDVTRSRDVPDFSDLVQQNLLSTTFVPLRAQRAWTYEAGARGRIDRLAWDVTLYRSDLRDELINFSTNPGXXLNIPAATFNTPRTVHQGVEAAVTLDLARDLXGXGDGLSVTQIWTHNDFRFVGDPVFGNNRIAGIPXDVLRTVMSYRHPSGFHIAPSLDWVPQGAFADHANTLRVPGYALLGVEAGIDFANGVSLFVDARNLTDARYVSDIAVVANAXXXXXXXAATAGGPXALAAFYPGSGRSVFXXXXXXXXXXXXXXXXXGGIRXXXXXXXXXXXXXXXXXXXXXXXXXASFXXXXXXXXXXXXXXXXXXXXXXXXXXX

SEQ ID NO: 10 - protein, 816 aa, synthetic construct
XXXXXXMSETESGPGSVTDADYRNLAETLPQLAWIAEADGTIVWYNQRWYDYTGTSLDEMRGWGWRTVHHPDHVAAVTERYRAAITLGRSWEDTFPLRGRDGSYRWFLSKALPHRDESGRILRWYGTNTDITVRRAVEERLRHSEQRFRALVDASAAVIWNTDAAGELMPPQVRWSTYTGQTEEAYQGWGWLDAVHPDDRGHAADAWAACVEARATYEVEYRLRRHDGAWRAMEVRGVPVLAEDGSLREWVGTCVDVTERKEAEEAVERARQAAEAANRAKSQFIANMSHELRTPLSAVIGYSEMLGEELEDIGQAALLPDLRKIEAAARHLLSLINDVLDISKIEAGRMTASAETFTVADLLRDVTDSTGSLVEKKGNRFVLDAGAAGEAGLGSMHQDQTKIRQCLLNLIGNAAKFTERGTITLTVRRHREAGADWLSFAVADTGIGLTEAQIDRLFERFVQADDSTTRQFGGTGLGLAITRAFCRTMGGDIGVASTPGAGATFTIRLPATLRPEDAXPPXXXTEAEAHTPVEPHEEHETVLLVDDDPAARELLQRFLEREGFHVRSANDGRAGLTLARALKPRAILLDVEMPRMDGWAVLHAVRNDPDLAGTPVIMTSVVAEQGLGQALGATDYFVKPIDWDRLKGLMERYRPAAPNEARVLVVDDDADARERLRRSLGREGWTVDEAENGRIALERVSQARPSLILLDLMMPEMDGFGFLRALRSRPDGDVPVVVLTAKEVTAAEKESLNRQADRVIAKGSMSLAEIGRQLRVLYARSATEPVPGQLQGLLDRLAEKDAGEPRXXXXXXXXXX

SEQ ID NO: 11 - protein, 110 aa, synthetic construct
MMMHVKMIAAAAAVVGGLGLAXSAGAAPLAPAGADTITGPAAVSTVAFGCGPGWAPGPYGRCRPIXXXYRRPRFYGPRCFFRPTPWGXXXXXXXXXXXXXXXPRRVCRXX

SEQ ID NO: 12 - protein, 562 aa, synthetic construct
MXIETVETAGRRRGADLLVEVLRSEGVRYIFGNPGTTELPLIDALTEAPDIAYILALQEATAVAMADGYAQGARRPAFLNLHTAGGLGHXMGGLVNSQVSGTPLVVTAGQQDLRHALTDPLLMGDLVAIADPVMKWAREVTSPDQIPILLRRAFHDXGAAPSGPVFLSLPMDVMEALTAVPAGETSTIDQRAVAGSLDRLAEKLAAIAPGRLALIAGDEIDASDASAQMVALADLLAAPVYGSSWPAHIPFPTAHPLWAGNLPTRADAIADILGRYDAVFALGGKSLITVLYSEVSAVPPGVQVFQLSADVRDLGRTYATXLSTVGDIRASLDALLPLLAPRLADRADAFAXLRAQAVTARAERRAKLAAAADAAFEDPVIAPLVAAREVARAVGAETTIVDEAPATLTHLRTFLDSPSAHQYAAMRGGVLGWGMPAAVGFSLGLDRAPVVCVVGDGAAMYSPQALWTAAHEKLPVTFVVINNAEYNILKTFMKGQAHYASVRANRFIAMDLTDPRIDFPALAASMGVPARRVTRAADIAPAIEAGIRSGGANLVEVVVRAT

SEQ ID NO: 13 - protein, 339 aa, synthetic construct
MLTVTRTPLRISLFGGGTDYPEYFERAPGAVVGAAIDKYIIIAALDLIGCQDYNYRLSYSRVEHCNNIXEIEHPVVREVLKHFDVNRRLDMSIISDLPAAGSGLGSSSAFTVGFLRTIYAILNQKPTKIELSKKAIEVEREILRENVGVQDQLHAAFGGINRFDFSGSAIRISPVQMSSAAIQQLNASMVLVHTGIARRATTTVAAQIAVTRARAIDKELSELYRLVEECVSLLEAGTSGWLXQLGEMLSASWRIKRTLSREVSNAVLDDLFEAIIASGAYGAKLCGAGGGGFFLALIDPDRLPALXERVAPLSVVPIGIDVDGSTLIYRQTRXXXXXX

SEQ ID NO: 14 - protein, 873 aa, synthetic construct
MSLRALRSSLVASASVLAGTVLPXAVQAQQSVTLGEISVVSTSPVGSGGGNSSXQAQIFNGPGPVPPAGSLGPSXXGVRLRGSEQPLYKIPSTVESVTASDITIDRASDNLTTTLARRTPGINVSDSQGNNNRVDITYRGFTASPVQGVPQGLAVYQNGVRINEAFGDIVNFDLIPPQAIQRIDVVTGNPVFGLNALGGAVNIQMKNGFTWQGTEISAWGGSDARTAGYLEYGKVSGPWSVYFTGDGLNDRGWRYESPSTIGRLYGDIGYRSQDSEFHLIGLAARSFFGAAAATPVDFTHRDPRAIFTYPQTTTTEVGTLQLTGRVDISPTWDLAGNAYFRRFSQTYVDGNDGNFENCSTRSSFRGNLCFEDDGFSPAAGQSQLAFRNQFLILGQXXQNQRIPFRAGIPYGTLDTTRTEATGFGGSLQAANRDRIFGLSNTFVVGGSIDAANYXFKSSSTLGVINPDLSITTDPSNPFYGNIPGLGTPQLRTAGALGIAPSSVNGSNLYMGLYTLDTLDVTDRLSLTAGXXXXARLNFARIQSEDLTGFSPDVTGTHYFNKINPVAGLTYRFFDALNLYGSYSESNRAPTPLELACANPDRPCLLPNSLVADPPLKQVTGRTYEVGFRGQLPNTYDGGIITYKIGAFRTDLANDILSLATPGNTARAYFVNVPSTQRQGIEVGGEYXXTADYLRVYANYALVDATFQFNGTLSSPNNPLAXXXXXXDDGAIQVRKGNVVPLVPTHQXKAGFDYFVTPNWQFGLYLQAFSSSYFRGDESNLNRKLPPYYVLNFQTKYQVTKNLEVFGLITNLTNNRYXTFGTFAEPGAVAGNLRISDPRTTTLAQPFSVYAGIRYAFGADPVPMSXPEPIIRKY

SEQ ID NO: 15 - protein, 262 aa, synthetic construct
XXXXXXXXXXXXMLEIDRLDAWYGPSHVLHGLSLEVRPGEILALVGRNGAGKTTTMKAVMGLIPKVAGSVRFLGEDLLGRPXHARFPXGLAYVPEDRRIVPGLTVRENLKLGLLRAXXXXXXXXXXXXXXXXXAXIKEGPAIAEIAETFPRLAERLDQTAVTMSGGEQQMLAIARAMIARPKMILLDEPSEGIMPVLVEEMGRLFVAMRXRGVTLLLVEQNVEWALNLADRAXIIDQGAVVHXSXAAALRADXEIQDRYCAV

SEQ ID NO: 16 - protein, 554 aa, synthetic construct
MNFVHRYLGESRAVSDADATRLGFVPDTLREPTYFAGTVARHLPFREAISALHHVVVSDLRFKPKDRTAYFAWLQAHEQELLAEALAEKDSLRAEIEALRAESRDIAARSDAVMRSFYDARKRYFDYLYRENLDAWIVLDPVITVHPDEIFFEAFSLDESSYGRLSCDHDTFARIGDMACGTTNIDYSHALYDEFQKIRSYRDTELAIDPTGFAVQTSGEAAYREDKIDLPDSWMRGFLQVSSAMAQPAHVVDLHPVDMHAILTRLAARRERHGPRSLRFLLEPGRPVSVLIEPWNERLTFRRSIYRGGEXAEIRLWGRRRLAILARTLPLARSVRLHLLGTGLPSFAVVDFGGLRFTLGLSGWTANDWSRAGQFDLLAPRADVDADTAARVFAALRRHHAADTGQLAAETGLDRSTVEAALGGYVQAGRAMFDLDKRVYRLRELTREPLAPGALRFASEQEAKADRFLAAGLVTLGPVEQAGDRRRLSGTVLDDGRSLTPAVELDSDDRMVGGSCQCGFYTHNRLTRGPCEHMLAVRRLVHAQVEGKPVRWQA

SEQ ID NO: 17 - protein, 484 aa, synthetic construct
MADPAPITGAPEPVKAAPGAFLTPLFTDRRVAAVLGLGFAQGIPFLLVYATQSAWLVQAKVPLATIGLMSELTIAYKLKFLWAPFLDRHDAPLIGRWLGRRRGWIVATQILVALALAGVAFGDPAHWLAWTVAFSLALGVAGATQDVVIDGWRITAAPPEQQALMSSWAEIGFRIGNLAAGAGALYLSDAYGWRVAYLCMAALMAPGTVAALLAPEPPVPETPATXXXXXXXXXXXXXXXXXXXXXXXXXXXXXXXGGFVETVWAPIRDLLAXXXRLGPLALPVLALVAGFRMPGYVSNAMAIPLFKTLGYTNTDIATVTKLFGFWIALGGTFLASAIIPRIGMMASLLIGTVTASASHLALAYLAWHGGHGGAAFWTFALTVGIDGFAYAFASIVLITYMSRLSATAHAASQYALLTSLCALPGSLLAGFSGFVIEWTGFAWFFVGTSLIGVPVALLCLLVARRHGPMEPAADAAGDAPNRAT

SEQ ID NO: 18 - protein, 250 aa, synthetic construct
MSDPSPAVSRAFVLAAGLGKRMRPVTATVPKPLVEVAGKALLDHALDRVAEAGIGTAIVNVHYLXDLIEGHLARRAEGXAXGPATTVSDERAALLETGGGIRKALPLLGDAPFVVLNSDSFWLEGPAXNLRRLIDTWDGDRMDGLLLVAPTATSLGYEGAGDFVMDPDGRLERRGERAVAPFIYAGVAILTPGLFADTPEGAFSLNLLFDRAIAAGRLFGMRLDGQWLHVGTPDAIRAAEERVRASARAP

SEQ ID NO: 19 - protein, 275 aa, synthetic construct
XXXXXXXXXXXXXXXXXXXXXXXXXXXXXXXXXXXXXXXXXXXXXXXXXXXXXXXXXXXXXXXXXMTVRTIIXXXGRDPAAAETSPQIHIXAEEIRRRRREQGLSLEXLAARSGVSRSMISKIERSEAVPSTVVLSRLAEALGVTFSRLMAPATEREVLLIPASRQPILRDEASGYLRRCISPVLPGRGIDWVLNTLPPGASTGEFTAHRRGVSEYIYVLRGRLRAVIGERAVVMEXGDSLYFEADAGHAFTNVGTEACEYFLVIDPSRVRXXXX

SEQ ID NO: 20 - protein, 550 aa, synthetic construct
MSDDEKPGWFGRLFGRKXXXXGAPESKSXEPVPAEDXXXXXXXEEXPXXXXXADESVTPDPLSPXASESEGQPXFTTAADDVAHVPPPVSEPAPDDPDKNRIPDELEGADLQPXXXXEPQPPSGXXXXEAPQPDEXXXXXXXXXESXXEXXAXXXEXAEKRNWWSRLTGGEXEXXXXXXXPAPAXXXXXXXXXXXXXXXXXXXXXXXAESEPPAEVDIQPVDSAAALASDAVSGAVEGSEKQGWWSRLTAGMRRTSSALSDRVTGLFTKRKLDATTLEDLEDALIQADFGVETATRMSEAVGKGRYEKGISPDEVRAILATEIERALEPVALPIEIDSAKKPYVILTVGVNGAGKTTTIGKLSLKFKAEGRSVMLAAGDTFRAAAIEQLRVWGDRTGTPVISRAQGSDAAGLAFDAFKEARENGTDVLLIDTAGRLQNKAGLMAELEKIVRVIRKLDPEAPHATLLVLDATVGQNALSQVELFSQAAPVSGLVMTKLDGTARGGILVALATKFGLPVHFIGVGEGVEDLEPFAARDFARAITGLPKEXXX

SEQ ID NO: 21 - protein, 544 aa, synthetic construct
XXXXXXXMRASLARLMPXXXXXXXXXSTLGRDLPASFVVFLVAMPLCMGIAMASGVPAERGLITGIIGGIVVGFLAGSPLQVSGPAAGLAVIVFEFVREHGIDALGPVLVAAGAIQLLAGALRVGGWFRAISPAVVHGMLAGIGILIVLAQIHVLTDALPKASGLDNLVAIPAAFFNFVSGPDGNRPGAVIVGLATIVAMIGWEKIRPAKLKLIPGALMGVLAGTLVAVVGSMDVKRVEVPENIFSAVTMPGXGDWSRLAEPAMILMAVTLAVIASAESLLSAAAVDRMHDGPRTQYNRELGAQGIGNILCGLAGGLPMTGVIVRSSANVQAGAATRASTILHGSWILAFLLVLPMVLKVVPTASLAGILVVTGWRLVSPAHAFHLHERYGLPTAAIWLATMVMVVATDLLTGVLTGLALSLLQVIPHXYLRGPLKIEGGAAQTVQGGAIQAVPELRLSGSATFLQLPHLNAALEXRTPEGSPVRLAAQDLRHVDHTCLEMIREWATRRAKTGSRVEVVGGGASGLHHSXLAMVAHAAXXXPKD

SEQ ID NO: 22 - protein, 99 aa, synthetic construct
MIVLIAAAIVSGLATATILAPVSALAALIIAPLAASASAILACIFIAWRNTRDDVGPPDLETQADAMVAVLCEVAQQGKIVPVAAPVRVRGHRSAXXXX

SEQ ID NO: 23 - protein, 256 aa, synthetic construct
XXXXXXXXXXXXXXXXXXXXXXXXXXXXXXXXXXXXXXXXXXXXMDITGEYRIXAPRAAVWAALNXXXXXXXXXXXXXDPEVLAXXXXXXXXXXXXXXXXXXRCIPGCKELTQASPEEMAAKVALKVGPVSATFAGTVXRFEDIRXPEGYTLVGQGNGGMAGFAKXXXGRAXVSLREEGXETVLTYEAKAEVGGKIASLGGRXXXXXLIQXTXXXSRKLADQFFGXFAAELGAPXPXAAAXAAXXXXXXXXXXXXX

SEQ ID NO: 24 - protein, 831 aa, synthetic construct
XXXXXXXXXXMSLFDLTGKXALITGSSRGIGRAIALRMAEHGARVVISSRKREACEAVVAEIEAAHGAGRAVAIPASISVKXELEXLVXETESRLGPVDVLVCNAASNPYYGPLAGISDAQFRKILENNVLSNHWLIQMVAPGMVARRDGAIVIVSSIGALKGSPVIGAYNVSKAADLQLARNYAVEYGXXXXXPANVRVNCLCPGLIRTDFARALWEDPEMLAATTDAAPLRRIGEPDEIAGAAVFLASAAGRFVTGQALVIDGGVTIAXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXR

SEQ ID NO: 25 - protein, 389 aa, synthetic construct
XXXXXXXXXXXXXXXXXXXXXXXXXXXXXXXXXXXXXXXXXXXMXIRSXAXRLTRRRAXAXLAAALALXXXXAXXXXXXXXXXXXXXAAPSXAAEPVVLRVGDQKGGNRSLLEISGYGKDLPYTIAWSEFPAAAPILEALNAGALDVGYTGDLSLLTAYAAGAPIKAIGGTKSDPRTQAILVRQDSPIRSAADLKGKRLAGTRGGWGQFLIDATLEKAGIKXEDATFAXLNPVDAKVALVAGSVDAWAVWEPYVSFATLKDKARXIADGXGLTPTITFIVASDSAIATKRAAVQDXLERLNKARLWSXXXLDHLDXYARNTAALTRMPEDVLRAAYTAQRTSPIGIDDGVVQEVQAASDRATRYGILXKRLDVXRVLDRSFTAAAXNXX

SEQ ID NO: 26 - protein, 1247 aa, synthetic construct
XXXXXXXXXXXXXXXXXXXXXXXXXXXXXXXXXXXXXXXXXXXMAKAXXXXXXXXXXXXXXXXXXXXXXXXXXXXXXXXXXXXXXXXXXXXXXXKARKPETAXXXXAKXXXXXXXXXXXXXXXXXXXXXXXXXXSKGRAEERTDAQLSALFDAAAXXXPAARDTRVISVRGAREHNLKNVDLTIPRDRFVVFTGLSGSGKSSLAFDTIYAEGQRRYVESLSAYARQFLEMMSKPDVDQIDGLSPAISIEQKTTSKNPRSTVGTVTEIYDYMRLLWARVGIPYSPATGEPIESQTVSQMVDRVLELPEKTRLYLLAPVVRGRKGEYRKEIAEFQKKGFQRLRIDGEYYAIDDVPKLDKKLKHDIDVVVDRIVVRDDIAARLADSFETALELADGIADIEFADAPEGEAPKKITFSSRFACPVSGFTIPEIEPRLFSFNNPFGACPTCGGIGHEMRIDPELVISDSALTLKRGAVGPWAKSTSPYYDQTLDALAKHFGFKTSVAWSALPEQAREVILFXXXXXXXXXXXGTGKESVRFDYNDGLRSYSVNKPFEGVIPNLERRYKETESDASREEIGRFMSATPCAACDGKRLKPEALAVKIDRQDIGQVTALSVREXXXXXXXXXXXXXXXXXXXXXXXXXXXXXXXXXXXXAHRWFSEISGXXXXXXXXXXXKLTDKQNEIAVRILKEIRDRLTFLVDVGLEYLTLARGSGSLSGGESQRIRLASQIGSGLTGVLYVLDEPSIGLHQRDNERLLGTLKRLRDLGNSVIVVEHDEDAILQADYVVDVGPGAGIHGGEIVAQGTPEELLKDPASLTAKYLTGELSVRTPKARRKPGRGMLRLVGARGHNLKNVTAEIPLGTFTCISGVSGGGKSTLIIDTLYKAAAKRLNGALEXXXXXXXXXXXXXHPAPFERIEGLEHLDKVIDIDQSPIGRTPRSNPATYTGAFTPIRDWFAGLPEAKARGYQAGRFSFNVKGGRCEACSGDGVIKIEMHFLPDVYVTCDVCKGKRYDRETLEVRYRNKSIADVLDMTVEEAADLFKAVPSIREKMETLARVGLHYVRVGQQATTLSGGEAQRVKLSKELSKRATGRTLYILDEPTTGLHFHDVAKLMEVLHELVDQGNTVVVIEHNLEVIKTADWVIDMGPEGGDGGGRVVAQGTPEEIAASTASHTGRFLREVLARXXXXXXRPAGKAVKXXXXDAAKDXXXXXXXXXXXXXXXXXXXXXXKAKAGAASGRRSNAAXXGRQAAE

SEQ ID NO: 27 - protein, 556 aa, synthetic construct
XXXXXXXXXXXXXXXXXXXXXXXXXXXXXXXXXXXXXXXXXXXXXXXAXAXLXAXXXALAXSXLXXXIGXXPAEARPAGYEGSAPSCFLTGGGALEICRDGSEAEGVLIAPAPCAFVRGVQILRLARSEAPGPLQAVFQGQTPVASXRRDEPCPERRATDSRDGGALRTGRTFEALVTTLPPSADRFWVDLPEERARXRGWSEPTPSAEGIGPRDPIAVTGRTWAGEAYSYVFLLGQERVGVTGGXDAEPERRGRRRRRRAXXXEXDAPATGPADVRRRVQIEARTLDFQTFEIRTRTADGYGLAWTPFEAXXXXGAGRGRRXDPPXPAAVVDEAGKPVTSACTAPAFETHGLAGSISVVDRTYHYVYTDVLPEDCGLAPEKRRTGLFLRTAQDLSGPKVWSTARKLAGPLPPGTLVRVARAKGMQRWAVSYTCQRPANAPGGPVADICLQYTADLNLDGIGALKLYADPVEAGRSXAYLGLRSGGDGSGRYDRSAHFWMTDAEGNLDTPAIYPNKAGFLTWLDRLAPTASGRDASSLYGRPVYWATWTVRXIGAQ

SEQ ID NO: 28 - protein, 247 aa, synthetic construct
XXXXXXXXXXXXXXXLXDXXXGRVYVDCXSCKXXXXXXXRXGRYXVXSLXERFGXDISTLXXDLLRXLTASCRYQRXPGAPPARKYEHLCLAAITLPXXXKXXXPVPPGVPYTIEVWXEXGGXIELHLAXIYPLXMAXAAFEXACXXWPXHEVTLRDRXRIVXKRERPXXXATXXXXXXXXXXXXXXXXXXXXXXXXXXXXXXXXXXXXXXXXXXXXXXXXXXXXXXXXXXXXXXXXXXXXXXXXXX

SEQ ID NO: 29 - protein, 229 aa, synthetic construct
  REGION 1..229: note = Consensus sequence
XXXXXXXXXXXXXXXXXVIHDXKXPVTRXLVLVRHGQSVANRSGLFTGLLDSPLTEQGRXEAVAAGRRLAERSWRFSDAFTSTLTRAVVSGRLILDTLGQPGLIPQRFAALDERDYGDLSGLDKTAADARWGAERIETWRRSYAEAPPNGESLRDTVARIVPCYLRSILPAVMGGDVLVVAHGNCLRALVMALDDLSPAEVEXLELATGSVRIYEFAADTTIEARWIDG

SEQ ID NO: 30 - protein, 169 aa, synthetic construct
LDEIAPLIEPQIPALRRYAVALLRDREAADDLVQDTLERALSAWSGRRRDGDLRAWLFTIERNLFLAAVRRRGRRGADLGAEALEQVPDPSADPGAALGARDVLAGLDTLPEEQRSVLLLVAVEDLSYAEAAQVLGVPLGTVMSRLSRARTRMRGFVETGRTGLLRRVK

SEQ ID NO: 31 - protein, 218 aa, synthetic construct
MATSPIWHVVSALAVAGTLAVHGGPAHAAPRQATAGALHFDDGGESYRGYRIEMAQDVPNAEIGQLRQAAEHQVDIVEATSLDEGTKAFLRRFPVVVXSGSGEXSHYSGGDXVTIAVEDPKDDRPILLHEYMHVYHFRKMPGGRNNPDILTFYGRAKDGGFYPAGAYLLKNQGEFFAMTASVYLHGRLAREPFTRDELRQKQPVYYRFLTRLFGPVGA

SEQ ID NO: 32 - protein, 123 aa, synthetic construct
MTVRNIAACVLLLCTGPALADTLPLRHGAYVSVGTDCKDPPNVALRTYDGGGLGSSKANDCRSRVLSKQGNVFEIEQDCRQFGGPKVERATERSTIRVDGPDRYTDLTXGXGESFRLCPGLKP

SEQ ID NO: 33 - protein, 2733 aa, synthetic construct
XXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXMALQXXADXXAAXLDAXEQXAAXLLDGXXQGLGALGEAAGQGFAXGVHQGAEXXGXALEXXXXXLGAXVDQXGERXLAXXERXGQXRXVGLDLXXMGXRPXVEGGXQXXXALVHEAXEGLVAVVEGXGQGVGALXXQXREXXXAAVDXLGEGIXAGVDXAGQXLXXXXXXXXXXXXAALXDXVEVXQXGVEXLXQGXXXXXXXXXXXXXXXXXXXXXXXXXXXXXXXXXXXXXXXXXXXXXXXXXXXXXXXXXXXXXXXXXXAXXXLGXXXQXXAAXLQEPLDXRAXLLEGXXQGXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXREAGVQQVXHXGGRRVEXXAXXLGXGXDXVGDVXAXXVHEVAQAXLGLLEQGXQALXAXVEGXGELALAGLDQXRXGGGXLLQXXAQGLXAXVXXGGEAAAAAXDXXVEGXLGLLEQXAQXLGXGXEGVGEAVAXAVDQXLQXAEXXVEQXAEXXGAXVQXAGGXXERAXQXXDEXXXXAVQXLXDXAXAXVQGXXQGAGVXVQHDADLVGXVVQQXVXXAHALVEQAADVXXAARQXVAQAALAXIEHPLGVXDGLLELGEXALAXLVDEXGERGAAXVEQAGELGDXXADXXEHXXALLVELXRHXXXALVDEAXEQVGAXGEGGXHXLGAGXQXXVGXXGALVHQXXXXLRAXRQXGLEXAGXLLDGAGEGXDALGHGXVEAAGXXXDXXHEVAGXXGQLXVXXAGARLDQADRLEGLXAEALVHEAGALVQXXXDXLQAAXDXXVEXXGAXLDXAGDLAXXAXDVXAEXXGAXXXXXXXXXXXXXXXXXXXXXXXLXXLHQAAXAXXXDPGHLXXXGXQXLVQGXGAXXXXXXXXXXXXXXXXXXXXXXXXXXXXXXXXXXXXXXXXXXXXXXXXXXXXXXXXXXXXXXXXXXXADMXVDLADQXGHGXGXXGGXLXXXXXXXGXAXLQXAEMGLDQAAGLAGXXRDXLGHGLAXXGEEXXXLLQXXAXGLAEGGGVAADPVGQDVAXGDGLLQGGEAAAQXLVDXXDPXVGGLGEXXAXXAEXVGDAXALARHGEHDVAAGAXDXGLGLVXVAGEQLGXXAAMXAEGXXXLADXXXEVGRDXXGGXXEXAGXXAGXGGEFXVEXXEGXAXXRDGGXAXXAEXXXXXVGGVAEXGXEDXGXXLDGXXDLXXGGAQXXQXXGEXXXGIXXXXLGXLAXLXGGXAXXXGXXXXXXXXXXXXXXXXXXXXXXXXXXXXXXXXXXXXXXXXXXXXXXXXXXXXXXXXXXXXXXXXXXXLXGAAVDLARQXLDXXXQXXXXXXGXXLEAXAQGREGXLQAXXXXXXPXXDXAAGXLGRRXDXVLQXRXXDXXGLVEAXXDAVHPLGEXAAAXXXGIXDPLGGXDDVVAHXXGAGXQXXXXAXXRXXQGXLXXXXAXXDXXXDXXAXXLDLGAQXXAXAXXVLXEGRAXLGEAXGDLGAXXXEALXXXXXXXXXXXGQXXAPXQEGXXQXRXGALEALXHRXXLXXXXXADAXXGLXQGXXDXAXXLGQXXGQGXXXXLEXAGXXXGAILEXAXQXXADXXEAXAHXXAGXRQXVDQXXAAXXHQXXXXVAXLAQXGGDXXAXXLQGAGHALAXXXXGRDDPVGXXIQXLXQXXVRAGDGAAHPLGVGDDXLALXXQLVXEGADADLVVGIGXLQGGDLAAHQGXQLAXPGEXXLDAVAHRRXLAAXGLRHGQXXVXXEALXLGQAHRDLAHXAGDEAHLLGAHXQHXXDXEQXHRXEQRRXXXXXLEXXEAXXDXXQVAXXLRPXDQXEXXDPXXAGXXGDXVGLXRGAHPQGLLQDADVAPVVVGHQRAVGRQEAALXAGXVQXXXXQVXGEXRVXXXXXXXXXXXXDVEAXRXXXXGGVXXAVEQRIGVXDIXQXXLXXXXXAVXXQGVEXQXLLDXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 34 - protein, 389 aa, synthetic construct
MLAMNYRGPYRVRVDRKPVPVCEHVEDAVIRVTRTCICGSDLHLYHGMVPDTRVGMTFGHEFTGVVEEVGSGVRNLAVGDHVLVPFNIACGRCHFCKQGLFGNCHEANAQATAVGGIYGYSHTAGGYDGGQAEYVRVPYADVGPCKIPXTMDLDDAVLLTDVVPTGYQAAEMGGIQRGDTVVVFGAGPVGIMAARCAWLFGAGRVIVIDHVEYRLDFARRYCPAEVYNFRSIGDPVVFLKKTTDSLGADVCIDAVGGDAAGSALHTLMGTKLKLEGGSAXALHWAINSVKKGGIVSIVGVYGPTGNLIPIGNVLNKGITIRANQASVKRLLPKLIXHIEAGRLNPKELITHKVPLEEAADAYHLFSAKLDGCIKPVLVPPTARXXXXXX

SEQ ID NO: 35 - protein, 213 aa, synthetic construct
XXXXMRNRPIDPSTVPGWGVDADPQNDPTYPMRDXARDDXXXXXXXXXXRGMNWXRPPXXXXQQRARVEVLTSIEYNXRPAVXGTSTPPRGVSGVIRRQAFRYSESQWAHWLMLMAADRVNVVEGVVDDLXRGRVPNVPAEMGARAELAHNRSGLAXKLALTGAAIGLGILVSRLARAQRRXXXXXXXXXXXXXXXXXXXXXXXXXXXXXXXX

SEQ ID NO: 36 - protein, 316 aa, synthetic construct
XXXXXXXXXXXXXXMPPLLALPFPAIDPVAVSIGPIEIKWYALAYIAGLVGGWYYARRLVXADSLWGVVKRPTLXDIDDLIVWVALGVVLGGRIGYVLFYNLPMYLXHPXEILAIRNGGMSFHGGFLGAILAXLLFARSRGLNGYTMLDIXAVVVPIGLFFGRIANFVNGELWGRAAPDFPYAVVFPTGGPVPRHPSQLYEAATEGLLLFIVMALAVRRFGFRKPGLLGGIFVLGYALARTFCEFFREPDRQLGFLFGXDVXXMGGGVTMGMLLCVPMXLVGLXYIVLAARGXTRPRKTXXEIXAXEAXQXXXXXX

SEQ ID NO: 37 - protein, 275 aa, synthetic construct
XXXXXXXXXXXXXMFIEAPELSSHSXIRHAFFTRQGGVSEGLYASLNGGLGSNDDPERVAENRARMCAQLGLPRDXNLVSLYQVHSAEVVTVEAPFPXAERPKADAMVTRVPGLALGIATADCGPILFADPENRVVGAAHAGWKGALXGVIEATVAAMEALGAERRSIVAVLGPTIAQASYEVGPDFVERFRAEXPGXERFFXXGXXRPGHAQFDLPGFILXRLAEAGIGEATALGLCTYADPERFYSFRRTTHRNEPDYGRLISAIALTPXXXX

SEQ ID NO: 38 - protein, 416 aa, synthetic construct
XXXXATPLXAELARXIRXSGPIGIDRYMALCLGHPVHGYYRTRDPXGXXGDFTTAPEISQMFGELVGAWAAXVXXXMGAXXXPDXXXLVELGPGRGTLMADALRALRAAXXGXXAXXEXHLVETSPVLRRLQAXRLAXAXXXXXXXXXXXXXXXXPTWHDSVDTLPDGPLIIIANEFFDXLPVRQFVRTXXGWCERRVGLXXXXXPEGXALAFGLXPEPXPXLXAXXXXXEAPXGAVLTLPSXGLALMRXLARRLXARGGALLAIDYGHXRPGFGDTXQAVAGHRFADPLAXPGEADLTXHVDFXALARAAAAEGAAIHGPVTQRDFLLXLGLAXRAERLKARATPDQAQAIDAAVXRLTDPXPRGMGALFKVLXVSXPALGPLPXLPPXXXXXXXPXXXXXXXXXXXXXXXXXXX

SEQ ID NO: 39 - protein, 400 aa, synthetic construct
XXXXXXXXXXXXXXXXXXXMGPGREAVDAAALLDGFSACLSGLGLPLARATTHAPTLHPSFRWVMRVWHPGTSSLALRRRHGIEGTPTFHGNTVEHVVETRTPLQCRLDGDGPLPFPVLGELRNEGLTDYLIAPLXAARGRMGAASWATARPGGFTPIEIETLLALVEPFSLLFEIKALDDMLGAVLSAYVGRDPARQILAGTVRRGDVRLMRAAMMLTDLRGFGELSDRQSPDHVVAALNRMFDAIVPAVEAEGGEVLKYIGDGLLAVFDADRDEAEARRAALRAAEAALDALATLRDGDRAAFEVGVALHVGEVAYGNIGGGDRVDFTAIGRDLNVLARVERLCKTYDTPLIATDTFLHGLAHALEPLGIVALRGFAERHALFGCRRTAPVEAPAVXX

SEQ ID NO: 40 - protein, 327 aa, synthetic construct
MTHIRTSLRAXLAGAALLLAQGQPGSAAGFDGAIKNNALALNAAGTVAAVSNSEESAVVVYDVAKGTVLRRLDGFVTPRNIVFAPDGARFYVSDSGTGRITVYETATGKEAGVLAAGPGAFGTVLSADGGKLYVNNEAASTLTVFDTKTMLADAVIPGFAQPRQGVKLSPDGKTVFVTNFLGDKITLVDTATNKITGEIAGFDKLRAISITKDGKTLFAANSGRNTIGVVDVAARKVTSEVEVGKDPYGAALTPDGRFVYSGNLKDNSLSVIDTGTLKVVATVTGLNEPRQAIAFSADNARAYVLNRDLSVAVVDRAKNAVVSTMKP

SEQ ID NO: 41 - protein, 123 aa, synthetic construct
MSEPAKPVPPDDPRVRLAEDRTVLAAERTFVAWLRTGLAFLGVGLAAQRFLREVLAVWPLKVLSLTLIXCALASFAGAAWRDRAIRARLAHAEIPMMPRILTVGIAALLIAISGLAATALLWA

SEQ ID NO: 42 - protein, 457 aa, synthetic construct
XXXXXXXXXXXXXXXXXXXXXXXXXXXXXXXXXXXXXXXXXXXXXXMRVAIVHYWLIGMRGGEKVVEALCDLYPEADIFTHAYAPQSMSPTIRAHRVRTSFIGRLPFATSRYKSYLPLMPMALEQLDLRGYDLIISSESGPAKGIIPPSDALHICYCHSPMRYVWNMYHDYRERTGLLTRLLMPPVAHYVRNWDAVSAGRVHEFIANSDTVARRIETYYRRQAKVIHPPVDTAAFEXXPDGERGDYHLMVGEMVRYKRPELAIQAFNRLGQPLVVIGGGEMLRELRSMAGPHIKILGPQPFEVLKHHYARCQALIFPGEEDFGIVPVEAMASGRPVVAFGKGGVTETVIDGVTGTFFHEQSVDALIDAVQRCRAIGVEPERLVRRAADFGVGRFADEISRFVDGVLARERLAAPRPXXXXXXXXXXXXXXXXXXXXXXXXXXPREPSRAYLVQXXXX

SEQ ID NO: 43 - protein, 522 aa, synthetic construct
VAXXXXXXXSPXAVTXTQXQAQARPXAPQXRLNPXEIRAIVYGLMTAMLLAALDQTIVATAMPTIGLDLGDAANLPWIVTAYLLASTAVTPLYGKLSDIHGRRIMLLIAIATFVLGSLACALAPTMVTLALARGLQGIGGGGLIALSQTILADIMSPKERARYQVVIAGVFVAASVAGPLLGGLFAQHLHWSLIFWINLPIGILAFALTNANLKRLPRHERRHRLDWPGAALMVAGSVTLLLALSWGGVRYPWGSAPVLALLAGAAVLGGGFAARLATAAEPLIPTEVLKDRVVYSATLAACFAMGTFIGLTIYVPIFLEGVIGLSASESGVALVPLMIGTVTGATLSGRSMLHFRHYKRVPLAMMXLSLACCATIAWEGRALPFWLMEVLFALLSMGIGTILPLSTIAIQNAVEPHQLGIATAAMNFFRSLGGALIVAAFGTIVLGGAAGGAGXXXGGHDVESLIRGADPAQLALTFRXVFLAACLGLLGAFTFLALMEERPLRERXSPRMGGEPXXGXPX

SEQ ID NO: 44 - protein, 334 aa, synthetic construct
MTLAILLSGQGGQHPXMFDLTADHPAAQXVFSAARPLLGGTDXXXXPRDLARAXXXXXXXXXXGGDXLHXNRTGQILCCVAALAAWRALXXXXXGRAIVAGYSIGDLAAWGVAGRXDXADILALAARRAEAMDAAXXXXXSGEGFGLAGIRGLTLDALDDLAXRHGCHLAIRNAADSGVVGGRRDALDALCRDAXAAGAQRAVXLXVHTPSHTPLLAAASEAFRDALAXVXLRRPPXGAPRLLSGLDGTTVFRPEDGLDKLARQISHTIDWAACLEACREYGADRVLELGPGHALATMARXALPXARVHALEEFRSVDGVADWLXRPXXXXXXX

SEQ ID NO: 45 - protein, 772 aa, synthetic construct
XXXXMSGPAPDXGRLTVIGVRHHSPACAGLVRRTIAALRPAFVLIEGPVDFNPHLPDLALGHDLPVAIFSFRADAXGSAASYTPFCAFSPEWQALEAGRAVGAQTLFCDLPAWDPAFGRRANRYADPHGARAEAAERALAAALGVADQDALWDVLAEAAPEAELPARLDRYFALLRPPGTDDPAEEARERFMGAYAAHALRAAGDRPVVLVCGGWHADAVRRHAAQADGTRPEPAPPEPDLRTGSYVVPYAYPRLDRFSGYAAGMPAPGYYERVAEAGLAPAADWAMTAITAALREAGQVVSTADRIAWRVHAEALARLRAHPAILRADLIDAALAALVKDALDRPPAWAAGGAAPGHPALAAMLRALTGRREGRLAPGTRQPPLVADVAERLRAADLEPGPARRSIDLDWAEPGDRARAHLLHRLXLLGLPGIARXEGPDRAEPGLPRERFTLVRHPHWLGALIEASLWGGTLEMAAAARITARVEAAPDSLAVLTGALSDALFAGLTLEGDLLARLSAGIAAAHDXAALGAAGAGIVRLYRFGDAFXAPARPALARLCAALAARALFVVEGIRDPRAGLGAIPLLLACRDLFREVGAEVXGLDELRXPFAAMLGRRLADPETPPALAGAALGFRVACGAAGSDPEAALSRLRRFGLPATLGDFLAGLFALAREEIAADATLASVEGLVAAWGDDDFLRALPSLRMAFAWFPPRERERIAVAILRRSGLGEARAEVEALAWMRQRARPADQAEALAREARVAARLARYGLT

SEQ ID NO: 46 - DNA, 1023 bp, Methylobacterium sp.
  source 1..1023: mol_type = genomic DNA
atgctgctgggaaccctggccgcgggtgtgacgcggccggccatggcggagcccgtcgtcgaggacgtccggatcgtcgacttcgactgggtcgacggcgcgcgccagcgaccggtgccggtacgcctgtactggccgaacacgtcgtcgctccggcgccgcatcccgctggtcgtgttctcgcacggtctcggacagtcccggaccggctacagctatctcggtcggcactggtcgtcccacgggatcgcgagcctgcacctgcagcacgtcggcagcgacagctccgtctggaccggcaatcccctggccctgctcgaccggatcgagcgggcggcgcaggaacgggaggcgatcgcccgggcgcgcgacctgcgcttcgccctggaccgcctcctcgttcaggacgggggcgccttcggagaccggatcgacccgcgccggatcgtggcggccggccattcctacggcgccaacacgactctgatcgccgcgggtgcgcgcgtcgtccgcgacgggcacgcccttcaggcgcgggatccgcggatcgcggccggcatcgtgatctcggcgccgcccttctacggcgagcgcgacctgcgggcggtcctgggcgccgtcgagatcccgaccctgcacgtgacggcgaccgaggatgtcatccagctgcccggtcgcacctcgccgttcagcgaccgcctcgccgtgtacgaggcgatcgcgacgccccggaagtccttggcggtcttccagggcggctcgcacagcatcttcaccgaccgtcccctcacgggcggtctgaacctgaacccgcaggtcaaggcggcgaccgccgccgggacgctggcctttctcgacctggccttccgcggcgatccggagccgctgcgcgcgtggtcctcgacctggcgaccgatcctcgccgtcgcgcccgcgggctacgcgagcgcgtcgctggcgcaacccgcgatagcgacccgcggaaagaagcggacgcgcgcccagcccgtggagccgctctga

SEQ ID NO: 47 - DNA, 1296 bp, Methylobacterium sp.
  source 1..1296: mol_type = genomic DNA
atgctgacacgcgtttccctcaccggttcgctcggcctggctctcctggccgccaccgccttcccggccctggcgcagcaggaccggggcggtgggctggcccaggccgagtgggcgcagaactacgattccgccgcgacgatgcgggtgcagcgctccaacacgccgatcctctcgccccagaccctggcggcgaccgagcagatggtcgagcgctaccgcgacatcgtggcccgcggcggctggcaggccgtgtccggcgccgagcgcctgcgcgtcggttccaagagcccggcggtgacggcgctccgccagcgcctgatcgtctccggcgacctcgacccggcggccggttcctcgccggtctacgattcctacgtcgaggccggcgtgcgccgcttccaggcccgccacggcctgaaccagaccggcgcgatgaacgtcaccaccgttcaggcgatgaacgtgccggccgacgtgcgcctgcgccagctcgagctgaacgcggtgcgcctgcgctcctattcgggcaatctcggcgagcgctacgtcatcgtcaacattccggcggccctcgtcgagacggtggacggcgaccacgtcgcgacccgccacgccgccggcgtcggcaagatcgaccgtcagtcgccgatcatgaacgccaagatccaggaggtgaacttcaacccctactggaccgtgccggcctcgatcatcaagaaggacctgatcccgaagatgcagaaggatccggcctacctgaccgacaacaagatccgcatcttcaacggcgatcgtgagatcccgccgtcccaggtcaactggcattccgacgaggcgacccgctaccgcttccgccaggatccgggcgtcgacctgaactcgatgggcttcgtgcggatcaacatcccgaatccgcacggcgtgtacatgcacgacacgccggccaagggcatcttcggcgacgatttccgcttcgtctcgtcgggctgcgtgcgcgtgcagaacgtgcgcgaatacgtctcctggatccttcagggcaccccgaacgccaacccggacacgatcgaggcgatcatccagggcggccagcgcgtcgatgcccgcccggtggcgccgatcccggtctactggacctacatcaccgcctggtcgaccccggacggcctcgtgcagttccgcgacgacatctacaagcgcgacggcgccggcgccccgaccgcctcgatggccgccgccgccgccggcccgcgcaacttcaaccccgatgcccccgacgacgaacagaccaactga

SEQ ID NO: 48 - DNA, 1236 bp, Methylobacterium sp.
  source 1..1236: mol_type = genomic DNA
atgaccgagatcgataccgacgccttcctggccgacctctatgctctgcgcgagatcggccggttccgcaccggcgttcaccgcccgaccttctcggccgccgacatggaatcccgccgctggctgatggccaagctggaggaatgcgggctggaggcatcgatcgacgggatcggcaatgtgctcgggcgccatcgcggccccggaccccacctcctggtcggcagccacatcgagacccagaacgaggccggctggctcgacggggcgctgggggtggtggccgggctggccctggcccgggccgggctgccggtcgacgtcgtcgccttcgcggacgaggaggggcacttctccggcggcttcctgggcagccgctcggcgatcggcgacctgaccgaggccgagatcgaggccgcccgcaaccgcaccgacgacacgcctttgcgcgcggccctcgaggccgccggactcgccggattgccgcggatgcggctcgatccggcccgctaccggggcttcctggaactgcatatcgagcagggcacgcagctcgaatccaccggtctgcatctcggtgtggtcagcggcatcgtggcgatctggcaattccagatcgtgttcgacggcaaccaggaccatgccggcgggaccaccatggccgagcgccgggatgcggggctctcggcggtacgcctgctcgcggcgatcgaccgggaattcccgaaactctgcggcccccgcagcacctggaccaccgggcggatcaccctggatcccggcggctacagcatcatcccgggccgcgccgaggtggccttccagttccgcgacgtgtcgatcccggtgctggagcgcatggaggcgtgcctcgaggctctggtgcgcgagtcgaaccggcgcgaacgttgccccgccaccctcacggccctgtcgaaagcgatccccgccccctgcgacccggacctcatgcgggccctctcagaggccgccgagcaggtctgccccggccgctggcaggtgatgccctcgggcgccggccacgacgcccagaacatcgcccggatcctgccggccgccatgctgttcgtcccgtcgatcggcggcatcagccaccattgggcggaggataccagcgatgcggacctggccttcggggtgcgcacgctcggcgcggcggcggctcgggtgctggcgactcgcaaggcaggggactcattcaggtcgctgcgatga

SEQ ID NO: 49 - DNA, 600 bp, Methylobacterium sp.
  source 1..600: mol_type = genomic DNA
atgcgcaggccagatcagcccgccctgacggccgccgcccacggccgcgtgtccatgcccttcctgccgacgctggccgtcgtggcaatcctgggcgtggcctcgctctactgcccgacctcgaagccgggactgcccggcgcccgggtcgaagcgggcgtgtccgccgctcccggacccacggatttcgcgccgatcgccgccgtcgcggcgccgggccgcccgcccgccgtgatcgcgttcgccgagcagtacccgctcgacgccgtgatcgcgcggacggggtccctgcccgcccgtccggccgtggccgcacgggcgaacgcccacgtcgcggccgcgggccggcgcgcctgccccggccggcgctgccctgagacgccccgcagcaacaccgatcccatggcgcccgcgcgcggggcggccgatgaggccgaggacgccctgctcccgtcgcaggccatgcccttcgcggcctcggtggtggagaccctggttccggccgcgcaagccgtcggcgacgcggcgaacctcgtgcgcagcagcgcgagggcggtccagggcacggtggccctggcggtggccgactgcctgcgctga

SEQ ID NO: 50 - DNA, 942 bp, Methylobacterium sp.
  source 1..942: mol_type = genomic DNA
atgtcgaacagttccgagccgactctcctgacggccgaccaagacctcgtactccgcttctggggcgtccgcggctcgacgcccgtcagcggcccgcaatacgccgaattcggcggcagcacgccctgcatcgaggtgcggtgcggccagcggatgttcatcgtcgacgcgggctcgggcatctacaatctcggacagggtcaccggaccgatctgccccgggaagtcgatctgctgttcagccacctgcatctcgaccacaccgcggggctccccttcttcaagccggcggttctcgactgcgaccgggtgatcaacacctattgcggcaacctcggcggcgaatcggccggccggaccctcgaccgcctgttcgcgccgccgctgttcccggttacgctcgacaggctgtgctgcaccttccgccaccacggcttcgaggcggggcagaccctgaccttcccggacgggacgcgggtcgccacgatcctgctgaaccatccgcagggctccgtgggctaccggttcgagcatgccggcaagcgcctgtgcctgatcagcgacatcgagcacagcgacccctggcccgatcccaacctcgccgccttcgtggccgacgccgacctgatggtctacgacggcatgttcaccgacggggagtatccgacctgccggggctggggccactcgacctggcagaagggcgtggaactcgcccggacggcgggcgtcaaggcgctcgggatcattcacctgcacccggcccattccgacacggcgctgcgcgacatggaggccgacatgcaggccgagatgccgaccgcattcatcgcgcgcgaatgtcagagcctcacggtcggggctccccgggcggtcggcggccgtccaaacgggcgtccggccgtggcgcgggagatgcgccgccggatcaaggtcgcctga

SEQ ID NO: 51 - DNA, 972 bp, Methylobacterium sp.
  source 1..972: mol_type = genomic DNA
atgaccttcccgcttcgcgccggcctcgccgccgccctcacgctcgcggccgcccccgccttcgcccatgccgtgctcggcgtgaaacaggcgagcccgaaccagacctatcgcggcgtggtccagattggccatggctgcgacggcaagccgaccaccggcgtcaccgtcacgatccccgagggcgtgatcgcggcaaagccgatgccgaagcccggctggcagctcgccaccaccaagggggcttacggccgcgcctatccgacccatcacggcagcgtctccgagggcgtgaagacgatggcctggaccggcggctcgctgcccgacgaccagtacgacgagttcgtgttccaggcccgcctcaccgacgcggtggcgccgggcggcacggtctatttcccggtgcgccaggattgcgacggcgcgaccgtcgattggagccaggtgccggccgccggccaggcggcccgcgacctgacctccccggcgccgggcgtgcgcatcgtggcggcggctgccgccgccgcgccggccgctcccgcctccacctcgatcaaggccggcgacctcaccatcgcgcagccctggatccgcgcgaccccgggcggcgccaaggtggcgggcggctacctcacggtgaccaataccggcaaggagcccgaccggctggtctcggcctcgatcccgctcgcaccccggggcgaggtgcaccagatggcgatggacaagggcgtggcgaagatggcgccggtggaagccggcctcgtcatcaagcccggcgagaccgtggtgctcaagcccggcggctaccacctgatgttcatggacctgaccggcccggtgaaggccggcgacaccctcgacggcaccctcaccttcgcccgcgccgggacggtgccggtgcgcttcgccgtcggcgccatcggggcgtcggcgccggaggccgcgggcgggcatcagcaccattga

SEQ ID NO: 52 - DNA, 180 bp, Methylobacterium sp.
  source 1..180: mol_type = genomic DNA
atgcatgcgatcggtacgaggcccgaacacgccatacccatggaccagatcgctcccgtcacgtcctctcgcacccgcaagaagcctcatccggctctcgtcgtcgcgtcgattgctctggccgtcgggctctggttcgtcatctacgcggtatccggccgcgtcatctggttctggtaa

SEQ ID NO: 53 - DNA, 1242 bp, Methylobacterium sp.
  source 1..1242: mol_type = genomic DNA
gtggacttcaccctctcccaatcccagacccactggctgacccgcgtccgcgccttcatcgccgacgcgatcctgccggccgccgccgcggtgtcggccgagcgcgcgcagagccgcgaaccctcccccacgatggagcggctgaaggagaaggcacgtgccgagggcctgtggaacctgttcctgccgccctccgccgagcacgacaccgacgcctatcgcggcgccggcctgaccaacctcgactacgccctctgcgccgaagagatgggccgggtcgggatcgcctcggaggtgttcaactgcgcggcgcccgacaccggcaacatggaggtgctgcaccgctacggcaccgcggcccagaaggaccggtggctgaagcccctgatggcgggtgagatccgctcggccttcctgatgaccgagccggaggtcgcctcctcggacgccaccaacatcagcacgtcgatccgccgcgacggcgaccactacgtcgtcgacggccgcaaatggtggtcgaccggcgtcggcgacccgcgctgcaagatcgcgatcctgatgggcaagaccgacccggaggccccccgccaccggcagcaatcgcagatcctcgttcccatggacacgcccggggtccgggtggagcgcctgctcccggtcttcggctacgaggacgcgcccaagggacacggcgaggtggtgctggagaatgtccgcgtgccggccgggaacctgatcctcggcgaggggcgcggcttcgagatcgctcaggggcgcctgggtccgggccgcatccaccactgcatgcgcaccatcggggccgccgaggtcgcgcttgaggccatggcgcggcgcctcgtctcccgggtcgccttcggcaagcggatcagcgagcagtcggtctgggagcagcgggtcgcggaggcgcggatcgacatcgagatgacccgcctcctctgtctgaaggcggccgacatgatggacaaggtcggcaacaagggcgccaagctcgagatcgccatgatcaaggtcgcggcgccgcgggtggcgctcaaggtgatcgacgacgccatccaggcccatggcggggccggcgtctcggaggatttcgggctcgccaagatgtacgctcatatccgcaccctgcggctcgccgacgggcccgacgaggtccacaaccggtcgatcgcccggctggaattcgggcgctacaccaacagaagcgaggcggagcgcgcatga

SEQ ID NO: 54 - DNA, 2139 bp, Methylobacterium sp.
  source 1..2139: mol_type = genomic DNA
atgccccgcctgttccgggccggccgatgcgccgcgctcctgctctccgcctgcgcgacgccggccgcagcgcaacctcagtcatcggctgcctcggtcaccctgtccgaactcagcgtcgtcggcgccgccgcgacggacgcgccgtcgggcgagacccgcggctcgctctcggtgccgagcgtcgaccgccagcgggccgccctcgcgggaaccgtcgggtccgtggccttcgtcgacgccgcgacgctccaggaccgctacgccaacacgcttcgcgacgtgctcaaggacgtgccgggcgtctacgtgcaggagcgctacgggcaggagctgcgcctgtcggtccgcggctccggcatcgcccgcgcctttcacctgcgcggcatcgagctgctgcaggatgggatcccgctcaacctcgcggacgggagcggcgacttctatcaggtcgatccgctcgccctgcgctcggtggaggtctacaagggcggcaacgccctcaccttcggggcaacgaccttgggcggcgccgtcaacgtcgtgacgcccacggcctacacggcgctggcgcccaacatcctgcgggtcgatggcggcagcttcggcacgatccgcgagcaggcccagatgtcccgcatcgacgggccgctcgacgtgctggtcaacgcgacgctgaccaactccgacggtttccgtgcccacgaggcgcaggccacgaagaacttcaacgtcaatatcgggtaccggatcgcccccgacatcgagacccgcttctatctcggcacctatctcaccgaccagaagctcccgggcaccctgaccctcggccagagcctcacgacgccgacgctggccaatcccgccgccatctcgggcaaccagtcccgcaaggtcgagacggagcgcatcgcgaaccgcacctcgtttctcctcgacgtgggcaagctcgacgtcgacacctgggcgatccacaagagcctgtaccatccgatcttccaggcgatcgaccaggatggctggacctacgggatcagcccgcactgggccgggaccttcgatctcggcggcttccgcgacgacgtcgtcctcgggctgcgcgccttcgcgggccagaactccgcgctgcaattcgtcaatgtccgcggacagcggggggcgcagacgctgaattccctccaaagcgcgtcgagcgtcgaggcctatggcgagaaccgcttctggttcctgccggacgtggcgctgatggccggcgcgaaggcgttctcgtcgaaccggaccttcagcgataagggcggcctgccgggcaacccgaggccgcgcttcgccgatgtgacctacgaggggctcaacccgaaggtcggcctgctgtggcagccgcggcccgacatccaggtcttcggcgacgtcgtccggtcccgggacgtgccggatttctccgatctcgttcagcagaacctcttgagcaccacgttcgtacccctgcgggcgcagcgcgcctggacctacgaggccggcgcccgcgggcgcttcggccccctcgccttcgacgtgacgctctatcggtcggacctgcgcgacgagctgatcaacttcgcgaccaatcccggcctcaacatcccggccgccaccttcaacacgccccgctccgtccaccagggcgtcgaggcggccgtcaccctggacctcgtccgcgatctcgccgggaccggcgacggggtcagcctgacccagatctggacccacaacgacttccggttcgtcggcgacccggtcttcggcaacaaccgcatcgccgggatcccggcggacgtgctgcggacggtcctgagctaccggcacccgagcggcttccacgtcgcaccgtcgctcgactgggtcccgcagggcgccttcgcggatcacgccaacaccttgcgggtgccgggctacgtcctgctcggcgtcgaggccggcatcgatttcgccgacggcgtcgccctgttcgtggatgcccgcaatctcacgaatgcccgctacgtctcggacatcgccgtggtcgccaacgccgccgcgacggccggcggagcgggcgcgctggcggcgttctatcccggcagcggccgcagcgtcttcggcgggatccgggccgcgttctga

SEQ ID NO: 55 - DNA, 2391 bp, Methylobacterium sp.
  source 1..2391: mol_type = genomic DNA
atgagcgagactgagagcggcccaggatccgtcacggaggccgattaccgcaacctcgccgagacgctgccgcagctcgcctggatcgccgaggccgacggcaccatcgtctggtacaatcagcgctggtacgactacacgggcacctccctggacgagatgcggggctggggctggcgcacggtccatcacccggaccacgtcgcggcggtgaccgagcgctaccgcgccgcgatcacgctgggccggtcctgggaagacacgttcccgctgcgcggccgcgacgggagctaccgctggttcctgtcgaaggcgctgccccaccgggacgaatccgggcggatcctgcgctggtacgggaccaacaccgacatcaccgtccgccgcgcggtcgaggagcgcctgcgccattccgagcagcgcttccgggcgctggtggacgcctcggcggcggtgatctggaacacggatgcggcgggcgagctgatgccgccgcaggtccggtggagcacctataccggccagaccgaggaggcctatcagggctggggctggctggacgccgtccatcccgacgaccggggtcacgcggccgatgcctgggccgcctgcgtggaggcgcgcgcgacctacgaggtcgagtacaggctgcgccggtacgacggggtctggcgcgccatggaggtgcgcggcgtgccggtcctcgccgaggacggctccctgcgggaatgggtcggcacctgcgtcgacgtcaccgagcgcaaggaggccgaagaggcggtggagcgcgcccgacaggccgccgaggcggccaaccgggcgaagagccagttcatcgccaacatgagccacgagctgcgcacgccgctctcggcggtgatcggctactccgagatgctcggcgaggagctggaggatatcggccaggccgccctcctgccggacctgcgcaagatcgaggcggccgcccgtcacctgctgtcgctgatcaacgacgtcctcgacatctcgaagatcgaggccggccgcatgaccgcctcggccgagaccttcacggtggccgacctgctccgggacgtctccgactccaccggatccctggtcgagaagaagggcaaccgcttcgtcctcgacgccggcgcggcgggcgaggccggcctgggctccatgcaccaggaccagaccaagatccgccagtgcctactgaacctgatcggcaacgccgcgaagttcaccgagcgggggacgatcagcctgacggtgcgacggcaccgggacgcgggggccgactggctgtccttcgcggtcgccgacaccggcatcggcctgaccgaggcccagatcgaccgcctgttcgagcgcttcgtccaggccgacgattcgaccacccggcagttcggcggcacgggtctcgggctcgccatcacccgcgcgttctgccggacgatgggcggcgatatcggcgtcaccagcaccccgggcgcgggcgcgaccttcacgatccgcctgcccgcgaccctgcgcccggaggacgcgccgccgaccgaggccgaggcgcacacgccggtggagccgcacgaggagcacgagacggtgctgctggtcgacgacgacccggccgcccgcgagctgctccagcgcttcctggagcgcgagggcttccacgtccgcagcgccaacgacggccgcgccggcctgacgctcgcccgggccctgaagccgcgggcgatcctgctcgacgtcgagatgccgcgcatggacggctgggcggtcctgcacgcggtccgcaacgaccccgacctcgccgggacgccggtgatcatgacctcggtggtggccgagcaggggctcggccaggcgctcggcgccaccgactacttcgtcaagccgatcgactgggaccggctcaagggcctgatggagcgctaccggcccgcggcgccgaacgaggcgcgggtcctcgtggtcgacgacgacgcggatgcccgggagcgcctgcgccggtcgctcggccgcgagggctggaccgtcgacgaggcggagaacggccggatcgccctggaacgggtcagccaggcgcggccgagcctgatcctgctcgacctgatgatgcccgagatggacgggttcggcttcctgcgggcccttcggtcgcggcccgacggcgacgtgcccgtggtggtgctcaccgccaaggaggtcacggccgccgagaaggagagcctgaaccggcaggccgaccgggtgatcgccaagggctccatgagcctcgcggagatcggccggcaactgcgcgtcctctacgcgcgttcggcgaccgagccggtgccggggcagctccagggtttgctggatcggctcgccgagaaggacgcgggagaaccacgatga

SEQ ID NO: 56 - DNA, 270 bp, Methylobacterium sp.
  source 1..270: mol_type = genomic DNA
atgatgcacgtgaagatgctcgcggcagccgcggccgtcgtcggcggcctcggcttcgcctccgccggcgccgcgcccctggcgccgctggccgccgatacggtcaccggggccgcgccgatcagcaccgtcgcctatggctgcggccccggcttcgcacccgggccctacggccgctgccgcccgatataccgccggccgcgcttctatggcccccgctgcttcttccgcccgacgcccttcggcccgcggcgcgtctgccgctactga

SEQ ID NO: 57 - DNA, 1689 bp, Methylobacterium sp.
  source 1..1689: mol_type = genomic DNA
atgccgatcgagaccgtggagaccgccggccgccgccggggcgccgacctcctggtggaggtgctgcgctcggagggcgtgcgctacatcttcggcaatcccggcaccaccgagttgccgctcatcgacgcgctcaccgaggcgccggacatcgcctacatcctcgccctgcaggaggcgaccgccgtggcgatggcggacggctacgcgcagggcgcccgccggcccgccttcctcaacctgcacacggcgggcggcctcggccacgccatgggcgggctggtgaattcccaggtctccggcacgccgctggtggtcaccgccgggcagcaggatctccgccacgccctgaccgacccgctgctgatgggcgacctcgtcgccatcgccgacccggtgatgaagtgggcccgcgaggtgacgagcccggaccagatcccgatcctgctgcgccgggccttccacgacgcgggggccgccccctccgggccggtcttcctgtccctgcccatggacgtgatggaggcgctgtcggcggtgccggccggcgagacctcgaccatcgaccagcgggccgtggcgggctccctcgaccgcctcgcggagaagctcgccgccatcgcgccgggccgcctcgcgctgatcgcgggcgacgagatcgacgcctcggacgcctcggcgcagatggtggcgctcgccgacctgctggcggcgccggtctacggctcgtcctggccggcccacatccccttccccaccgcgcacccgctctgggccgggaacctgccgacccgggccgacgccatcgccgacatcctcgggcgctacgacgcggtgttcgcgctcggcggcaagtcgctgatcaccgtgctctactcggaggtctcggcggtgccgccgggggtgcaggtgttccagctctcggcggacgtgcgcgacctcgggcgcacctacgccacctgcctgtcgacggtgggcgacatccgcgcctccctcgacgccctgctgccgctcctcgcgccccgcctcgccgaccgggccgacgccttcgcgggcctgcgctccggggcggtgaccgcccgggccgagcgccgggcgaagctcgccgccgccgccgacgcggccttcgaggatccggtgatcgcgccgctggtcgccgcccgggaggtcgctcgcgcggtcggggccgagaccaccatcgtggacgaggcgcccgcgacgctcacccacctgcgcaccttcctcgacagcccctccgcgcaccagtacgcggcgatgcgcggcggcgtgctcggctggggcatgccggccgcggtgggtttctctttgggacttgatcgtgcgcctgtcgtttgtgtcgtgggcgacggcgccgcgctatactcgccgcaggccctgtggaccgccgcgcacgaaaagctgccggtcaccttcgtggtgatcaacaacgccgagtacaacatcctcaagaccttcatgaagggtcaggcgcactacgcctcggtgcgcgccaaccgcttcatcgccatggacctcaccgacccgcggatcgacttcccggcgctggccgcctcgatgggcgtgcccgcccggcgggtgacccgggcggccgacatcgccccggcgatcgaggccgggatccggtcggggggcgccaacctcgtggaggtggtggtgcgggcgacctga

SEQ ID NO: 58 - DNA, 474 bp, Methylobacterium sp.
  source 1..474: mol_type = genomic DNA
atgaattctgccgccattaagcaactcaacgagtcgatggtgttggtccatacaggaatcgcgcgccgcgcgacgacgacggtcgccgcgcagatcgcggtcacccgcgcgcgcgcgatcgacaaggaattgtccgaactctaccgcctcgtcgaggaatgtgtctcgctgctggaagcgggaacgagcggctggctcggccaactcggcgagatgctctccgcctcgtggcgcatcaagcgaaccctgtcgcgtgaggtctcgaacgcggtgctcgacgatctgttcgaagcgatcatcggggcgggggcttacggggcaaagctgtgcggcgcgggcggtggcggcttcttcctcgctctgatcgatcccgatcggctttcggccctgatcgagcgggtcgcgccgctgtccgtcgtgccgatcggcatcgatgtcgacgggtccaccctgatctatcgccagacgcgctga

SEQ ID NO: 59 - DNA, 2571 bp, Methylobacterium sp.
  source 1..2571: mol_type = genomic DNA
atgtcactgagggcgcttcggagcagcctggtcgcgtcggcgtcggttctgggcggggcgatgccctgcgccgcgcaggcgcagaatgccgtgacgctgggtgagatcagtgtcgtctcgaccacgccggtggggtcgggcggcggcaattcgagccgcgtccagaacctgaacggcccgggcggcctggagcccgccggctcggccccgccgccgatcggcccggtccgcctgcgcggcagcgagcagccgctctacaagatccccagcacggtcgagaccgtcacggccagcgacatcacggtcgatcgcggcaccgacaacgtcgtggcgaccctcgcccggcgcacccccggcctcaacctgtcggattcgcagggcaactcgaaccgccccgacgtcacctatcgcggcttcaccgtctcgccggtccagggcgtgccgcagggcctggccgtctaccagaacggcgtgcgcatcaacgaggccttcggcgacacggtcaatttcgacctgatccccccgcaggcgatccagcggatcgacgtggtgaccggcaacccggtcttcggcctcaacgcgctcggcggcgcggtcaacatccagatgaagaacggcttcacctggcagggcaccgagatctcggcctggggcggctcggatgcccgcaccgccggctacgtggagtacggcaaggtctccggcccgtggagcgtctacttcactggcgacggcctgaacgaccgcggctggcgctacgagagcccgagcacgatcggccggctctacggcgatatcggctaccgctcgcaggattcggagttccacctgatcgggctcgccgcccgcagcttcttcggcgccgccgccgcgacgccggtggacttcactcaccgcgacccgcgggcgatcttcacctacccgcagaccaccaccaccgaggtcggcaccctccagctcaccggccgggtcgacgtgtcgccgacctgggatctcgccggcaacgcctatttccgccgcttcagccagacctatgtcgacggcaacgacggcaacttcgagaattgcagcacccgctccagcttccgcggcaacctctgcttcgaggatgacggcttcagcccggccgccggccagacgcagctcgccttccgcaatcagttcctgatgctgggccagcagaatcagcgcatcccgttccggtccggcatcccgtacggcacgctcgacgtgaccaacacggaggccacgggcttcggcggatcgatccaggcggccaaccgcgaccgggtgttcggcctgccgaattccttcgtggtcggcggcagcatcgatgccgcgaactattcgttcaagtcgtccagcaccctgggcgtgatcaatccgaacctgtcgatcaccaccgatccgagcaacccgttctacggcaacatcccgggcctgggcacgacccagctccggaccgccggggcgctcgggatcgccccgagctcggtcaacggctcgaacctgtatatgggcctctacaccctcgacaccctcgacgtgaccgaccggctgtcgctgacggcgggcgcacggctcaacttcgcccgcatccagagcgaggaccttaccggcttctcgccggacgtgaccggcacgcattactacaacaagatcaacccggtcgccggcctgacctaccgcttcaccgacgcgctgaacctctacggcagctactcggaatcgaaccgcgcgccgacgccgctggagctggcctgctccaacccggaccggccgtgcctgctgccgaattcgctggtcgccgatccgccgctcaagcaggtgaccgggcggacctacgaggtcggcttccgcggcgcgctgccgaacacctatgacggcggtgtgatcagctacaagatcggcgccttccgcaccgacctgtcgaacgacatcctgtcgctcgccaccccgggcaacaccgcccgcgcctacttcgtgaacgtgccctcgacccagcgccagggcatcgaggtcggcagcgagtacacggccgattacctgcgggtctacgccaactacgcgctgatcgacgccaccttccagttcaacggcacgctctcctcgcccaacaacccgctctcggatgacggggcgatcgaggtccgcaagggcaacgtcgtgccgctggtgcccacgcaccagttcaaggccgggttcgattacttcatcacccccgactggcaggtcggcctctaccttcaggccttctcgtcgtcctacttccgcggtgacgagtcgaacctgaaccgcaagctgccgccctactacgtcatgaatttccagacgaagtatcaggtcaccaagaacctcgaggtcttcggcctcatcaccaacctgaccaacaaccgctacgccacgttcggaaccttcgccgagccgggcgccgtggccggaaatctgcggatcagcgatccgcggaccacgaccctgtcgcagccgttctcggtctatgccggcatccgctacgccttcggggcggatccggtgccgatgtcggccgagccgatcatccgcaagtactga

SEQ ID NO: 60 - DNA, 741 bp, Methylobacterium sp.
  source 1..741: mol_type = genomic DNA
atgaatgccgtcacgcccgcgccgctcctggcggtcgagaacctacgcgcgtggtacggtcactcgcacatcctccagggcctctcgctcgaggtccgggccggcgagatcgtgaccctggtcggccgcaacggcgccggcaagacgacgacgctgaagtcgatcatgggcctcgtgccgaagcgcgaagggcgcgtcgtcttcgcgggcaccgacatcctggcccggccggcctacgagcgcttccaccgcggcctcgcctacgtgccggaggagcggcgcatcgtgcccgggctgacggtggaggagaacctgcgcctcggcatcctggcggcgagaaacgagcgggtgtcgcgccgcgacgaggcgcggcggatcgaggcgctcgccgagaccttcccgcgtctcaagcagcggctgaagcaggaggcgatcacgatgtcgggcggcgagcagcagatgctcgcgattgcccgcgcgctcatggccgagccggtgatggtgctcctcgacgagccctccgaaggcatcatgccgatcctggtcgaggagatgttcgagcagttcgtggcgatgaagcgggcgggcaccaccatcctgctcgtcgagcagaacgtcgaactcgccctcgacatctcggacagggtttacatcatcgacggcggcgcggtcgtctaccacgccccggcagccgagctgcgggacgatcccgacatccaggcacgctactgcgccgtctga

SEQ ID NO: 61 - DNA, 1665 bp, Methylobacterium sp.
  source 1..1665: mol_type = genomic DNA
atgaacttcgtccatcgctacctcggcgagagccgggccgtctcggatgcggacgcgacccggctcggcttcgtccccgacaccttgcgcgagccgacctacttcgccgggacggtggcgcgacacctgccgttccgggaggcgatctcggccctgcaccacgtcgtggtgtcggacctgcgcttcaagccgaaggaccgcaccgcctatttcgcgtggctccaggcccacgagcaggaactcctggccgaggccctcgccgagaaggacagcctgcgcgccgagatcgaggcgctgcgcgccgaacaccgggacatcgcggcccgttccgacgcggtgatgcggccgttctacgacgcgcggaagcggtacttcgactacctctaccgggagaatttcgacgcctggatcgtcctcgacccggtcatcaccgtccatccggacgaggtcttcttcgaagcgttcagcctcgacgaatcgagctacgggcgcctgtcctgcgaccacgacaccttcgcgcggatcggcgacatggcctgcggcacgaccaacatcgactacagccacgcgctctacgacgagttccagaagatccgcagctaccgcgacaccgagctggcgatcgacccgaccggcttcgcggtccagacctccggcgaggccgcctatcgcgaggacaagatcgagctgccggattcctggatgcggggcttcctgcaggtgtcgagcgccatggcgcagcccgcccacgtggtggacctgcacccggtcgacatgcacgcgatcctgacccgcctcgccgcgcgccgggagcgccacggcccgcgcgccctgcggttcctgctcgagcccgaccggccggtcagcgtgctgatcgagccgtggaacgagcggctgaccttccggcgctcgatctaccgcggcggcgaggccgcggagatccgcctgtggggccggcggcgcctcgccatcctggcccggaccctgccgctcgcccggtcggtgcggctgcacctcctcggcaccggcctgccgagcttcgcggtggtggatttcggcggcctgcgcttcaccctcggcctgtcgggctggaccgccaacgactggtcccgggccgggcagttcgacctgctcgccccccggggggaggtcgaggccgacaccgccgcccgcgtcttcgcggcgctccggcgccaccacgccgccgggaccgggcaactcgccgccgagaccggcctcgaccggtcgaccgtggaggcggcgctcggcggctacgtgcaggcgggccgcgccatgttcgacctcgacaagcgggtctaccgcctgcgggagctgacccgggagccactagcgccgggcgccctgcgcttcgcctccgagcaggaggccaaggccgaccggttcctggcggccgggctcgtcaccctcgggcccgtcgagcaggcgggggaccggcggcgcctgagcgggaccgtgctggatgacggccggagcctgacgcccgcggtggagctcgattccgacgaccgcatggtcggcggcacctgccagtgcggcttctacacccacagccggctcacccgcggcccctgcgcgcacatgctggccgtgcgccgcttcttccacgcccaggtcgagggcaagccggcgcggtggcaggcgtga

SEQ ID NO: 62 - DNA, 1353 bp, Methylobacterium sp.
  source 1..1353: mol_type = genomic DNA
atggcagacccggcgccgatcaccggcgcgcccgagccggtgaaggccgcgccgggcgccttcctcacgcccctgttcaccgaccggcgcgtcgccgcggtgctggggctcggcttcgcgcagggtatcccgttcctgctcgtctacgcgacgcagtcggcgtggctcgtccaggcgaaggtgccgctcgccacgatcgggctgatgagcgagctcaccatcgcctacaagctcaagttcctctgggcccccttcctcgaccgccacgacgcgccgctcatcggccgctggctcgggcggcgccgcggctggatcgtcgccacgcagatcctcgtcgccctggcgctggcgggcgtcgccttcggcgacccggcccactggctcgcctggacggtggcgttctccctggcgctcggcgtcgcgggcgccacccaggacgtggtgatcgacggctggcgcatcaccgccgcgccgcccgagcagcaggcgctgatgtcgtcctgggccgagatcggcttccggatcggcaacctcgcggccggcgccggcgcgctctacctgtccgacgcgtatggctggcgcgtggcctacctgtgcatggccgcgctcatggcacccggcacggtcgcggccctgctggcgcccgaaccccccgtccccgagacccccgccaccggcggcttcgtcgagaccgtcagggcgccgatccgcgacctcctcgcccggctcgggccgctcgccctgccggtgctggcgctggtcgcgggcttccggatgcccggctacgtctcgaacgccatggcgatcccgctgttcaagacgctcggctacaccaacaccgacatcgcgacggtgacgaagctgttcggcttctggatcgcgctcgggggcaccttcctggcgagcgccatcatcccgcggatcggcatgatggcgagcctgctcatcggcaccgtgacggcctcagcctcccacctcgcgctggcctacctcgcctggcacggcggccacggcggcgcggcgttctggaccttcgcgctgaccgtcgggatcgacggcttcgcctacgccttcgcgtcgatcgtgctgatcacctacatgtcgcggctctcggccaccgcccacgcggcgagccagtacgccctgctgacctcgctctgcgccctgcccggcagcctgctcgcggggttctcgggcttcgtgatcgagtggaccgggttcccatggttcttcgtcggcacgtccctcatcggcttgccggtggccctgctctgcctgctggtcgcgcgccggcacgggccgatggagccggcggccgacgcggccggggacgcgccgaatcgggccacctga

SEQ ID NO: 63 - DNA, 741 bp, Methylobacterium sp.
  source 1..741: mol_type = genomic DNA
atgagcgagcccgcccccgccgtcacccgtgcgttcgtgctggccgcgggcctgggcaagcgcatgcggccggtgaccgccacggtgccgaaacccctggtggaggtggccggcaaggcgctcctcgaccacgccctcgaccgggccgccgaggccgggatcgagaccgcgatcgtcaacgtccactacctgcccgacctgatcgaggggcacctcgcccgccggacgggcgggcccgccatccaggtctcggacgagcgcgccgcgctgctcgagaccggcggcggcattcgcaaggcgctgccgctgctcggcgacgcgcccttcgtggtgctgaactcggattcgttctggctcgaaggcccggcccccaatctccgccggctgatcgacgcctgggacccggcgcgcatggacgccctgctgctggtggcgcccaccgccaccagcctcggctacgacggcgcgggggatttcctgatggacgcggacggccgcctggagcgccgcggcgagcgcgaggtcgccccgttcatctatgcgggcgtggcgatcctgcagcccggcctgttcgccgacacgccggagggggccttctcgctcaacctgctgttcgaccgggcaatcgcggccgggcgcctctccggcatgcgcctcgacggccagtggctccatgtcggcaccccggacgcgatccgcgccgccgaggagcgcgtgcaggcgagcgcccgggccccgtga

SEQ ID NO: 64 - DNA, 609 bp, Methylobacterium sp.
  source 1..609: mol_type = genomic DNA
atgacggcgaggacgatcatcggggcggacccggcagcgacggagacgtccccgcagatccacatcgccgaggagatccggcggcgccgcagggagcagggcctgtcgctggagacgctggcggcgcgctcgggcgtcagccgctcgatgatctccaagatcgagcgctccgaggccgtgccgtcgacggtggtgctgtcccgcctcgcggaggcgctcggcgtcaccttctcgcggctgatggcgcccgcgacggagcgcgagatcctggtgatcccggcgagccgccagccgatcctgcgcgacgaggcgtccggctacctgcggcgctgcatctcgccggtcctgcccgggcggggcatcgactgggtgctcaacacgctgccgccgggcgccagcaccggcgagttcaccgcccatcgccgcggcgtgtccgagtacatctacgtcctgcgcggccgcctgcgggcggtgatcggcgagcgcgcggtcatcatggagaccggcgacagcctgtatttcgaggccgatgccgggcacgccttcaccaacgtcggcacggaggcgtgcgagtacttcctggtgatcgacccgtcgcgcgtccgctga

SEQ ID NO: 65 - DNA, 1467 bp, Methylobacterium sp.
  source 1..1467: mol_type = genomic DNA
atgagcgacgacgagaagcccggctggttcggacgcctgttcgggcgaaagggcgcgcccgaatccaagtcccccgagccggtgccggccgaggatgcagcggccgaggaaacgccggccgaggaatcggtcacgccggatccgctgtcgcccgcctccgaatccgaagggcagccggtcttcaccaccgcggcggacgacgtcgcccgcgtgccgccggcggtgagcgagcccgcgccggacgatcccgacaagaatcgcatccccgatgaactggagggtgccgacctccagcccgagccgcagccgccatccggcgaagcaccgcagcccgacgagatttctgccgagagcgaaccggcggccgcccccaccgagacggcggagaagcggaactggtggtcgcgcctgacgggcggcgaggaggagacgcctgcgcccgcggccgaatccgaaccgccggccgaggtcgatatccagccggtcgattccgcggcggcgctcgcgagcgatgcggtgtccggcgccgtcgagggcagcgagaaacagggctggtggtcacgcctgaccgccggaatgcggcgcacttcctccgcgctgtcggaccgggtgacgggcctgttcaccaagcgcaagctcgacgccacgaccctcgaggatctggaagacgcgctgatccaggccgatttcggcgtggagaccgcgacgcgcatgtcggaagcggtcggcaagggccgctacgagaagggcatctcgcccgacgaggtgcgcgccatcctcgccaccgagatcgagcgggcgctggagccggtggcgctgccgatcgagatcgattcggccaagaagccctacgtgatcctgacggtcggcgtgaacggcgccggcaagacgacgacgatcggcaagctctcgctcaagttcaaggccgaggggcgcagcgtcatgctggcggccggcgacacgttccgcgcagccgcgattgagcagttgcgggtgtggggcgatcgcaccggcacgccggtcatcagccgggcgcagggctcggatgcggccgggctcgccttcgacgcgttcaaggaggcgcgggagaacggcaccgacgtgctcctgatcgataccgccggccgcttgcagaacaaggccgggctgatggcggagctggaaaagatcgtccgggtcatccgcaagctcgatccggaggcaccgcatgccaccctgctggtgctcgacgccaccgtcgggcagaacgcgttgagtcaggtggagttgttctcgcaggccgcacccgtgtcgggcctcgtgatgaccaagctcgacggaacggcgcggggcggtattctggtggcgctcgccacgaagttcggcctgcccgtgcacttcatcggggtcggcgagggcgtggaagacttggagccgttcgccgcgcgggactttgcccgggcgatcaccggccttcccaaggagtag

SEQ ID NO: 66 - DNA, 1569 bp, Methylobacterium sp.
  source 1..1569: mol_type = genomic DNA
atgcgcgcctccctcacccgactgatcccgccaaccctggcacgggaccttcccgcgtccttcgtcgtcttcctcgtggcgatgcccctctgcatgggcatcgccatggcctccggtgtaccggccgagcgcggcctgatcaccggaatcatcggcggtatcgtcgtcggcttcctcgccggctcgccgcttcaggtcagcggcccggccgcgggcctcgccgtcatcgtcttcgagttcgtgcgtgagcacggcatcgacgcgctgggccccgtcctcgtcgctgcgggcgccatccagctcctggccggcgcgctgcgggtcggcggctggttccgggcgatttcgcctgctgtggtccacggcatgctcgccggcatcggcatcctgatcgtgctggcacagatccacgtgctgaccgacgcgctgcccaaggccagcggcatcgacaacctcgtcgccatccccgccgcctttttcaacttcgtctcgggtcccgaaggcaaccgtcccggtgccgtcatcgtcggcctcgtcacgatcgcggcgatgatcggctgggagaagatccgtccggcccggctgaagcagttgccgggtgccctgatgggcgtcgtcgcgggcaccctcgtcgcggtgttcggcgacatggcggtgaagcgcgtcgaggtgccggagaacatcttctcggccgtcacggtgccggcgatgggcgattggagccggctgaccgagcccgcgatgatcgtcatggcgatcacgctggcggtcatcgccagcgcggagagcctgctctcggcggcagcggtcgaccggatgcacgacggtccgcgcacgcagtacaaccgggagctcggagcccagggcatcggcaacgtgctctgcggattggccggcggcctgccgatgaccggcgtcatcgtccgctcctcggccaacgtgcagtccggcgccgcgacccgcgcctcgacgatcctgcacgggagctggatcctggccttcctgctggtcctgccgatggtgcttcagctggtgcccacagcctctctcgcgggcatcctggtggtgacgggctggcgcctcgtcagcccggcccacgcggtccacctgcacgagcgctacggactcgccacggcggcgatctggctcgcgaccatggtgatggtcgttgccaccgacctgctcacgggtgtgctcacgggcctcgcgctcagcctcctgcaggtgatcccgcacttcgtccgcggcccgcttaagatcgagggcggcgcgtcggagacggtgcagggcggagcggtgcaggcggtgccggagctgcggctctcgggatcggcaaccttcctgcagttgccgcacctcaccgatgcgctggagcgcacgccggaaggcaggccggtccggctggcggcggaggacctgcgccacgtcgaccacacctgcctggagatgatccgggaatgggcggcgcggcgggcgcgatccgggtcacggatcgaggtcgtcggcggtggacgaagcggtctccagcacagccttgccatggtggcccacgcggcacccaaggactga

SEQ ID NO: 67 - DNA, 288 bp, Methylobacterium sp.
  source 1..288: mol_type = genomic DNA
atgatcgtcttgatagccgcagccatcgtcagcggtctcgcgacagccacgatcctggcgccggtgagcgccctcgccgcgctgatcatcgcacccctggcagccagcgcgtcggccatcctcgcttgcatcttcatcgcgtggcgcaacacgcgcgacgatgtcgggccgcccgacctcgagacgcaggccgacgcgatggtggcggtcctctgcgaggttgcccagcagggcaagatcgtcccggtcgccgcgccggtccgggtccgcggccaccggtcggcctga

SEQ ID NO: 68 - DNA, 477 bp, Methylobacterium sp.
  source 1..477: mol_type = genomic DNA
atggatatcacaggcgagtaccgcatcgcggcgccgcgcgcggccgtctgggccgccctgaacgacccggaggtgctcgcccgctgcatccccggctgcaaggagctgacgcaggcctcgcccgaggagctggccgccaaggtcgccctgaaggtcggcccggtctcggcgaccttcgccggcaccgtgcggttcgaggacatccgcgccccggagggctacaccctggtcggccagggcaacggcggcatggcgggcttcgccaagggccgcgccgtcgtctcgctccgcgaggagggcgcggacaccgtgctgacctacgaggccaaggccgagatcggcggcaagattgcctcactcggcgggcgtctgatccaggggacctcgcgcaagctcgccgaccagttcttcagcaccttcgccgccgaactcggagcgcccgcgcccgcctccgaagcagccgtcgccgccccctga

SEQ ID NO: 69 - DNA, 771 bp, Methylobacterium sp.
  source 1..771: mol_type = genomic DNA
atgtcgctgttcgatctgaccggcaagaccgccctcatcaccggctcgtcgcggggcatcggacgggcgatcgccctgcggatggccgagcacggcgcccgggtggtgatctcgtcgcgcaagcgcgaggcctgcgaggcggtcgtggccgagatcgaggccgcgcacggcgccggccgggcggtggcgattccggccagcatctcggtgaaggaggagctggaaaccctggtcgccgagaccgagagccggctcggcccggttgacgtgctggtctgcaacgccgccagcaatccctattacgggccactcgccggcatctccgacgcgcagttccgaaaaatcctcgagaacaacgtcctgtcgaaccactggctgatccagatggtcgcccccggcatggtggcgcggcgggacggcgcgatcgtcatcgtctcgtcgatcggggcgctcaagggctcgccggtgatcggcgcctacaacgtctcgaaggccgccgacctccagctcgcccgcaactacgcggtggagtacggccccgccaacgtgcgggtgaactgcctgtgcccggggctgatccgcaccgatttcgcccgggccctgtgggaggatcccgagatgctcgccgccaccacggacgccgcgcccctgcgccggatcggcgagcccgacgagatcgccggcgcggccgtgttcctggcctcggcggccggccggttcgtgaccggccaggcgctcgtgatcgacggcggcgtgaccatcgcccgatga

SEQ ID NO: 70 - DNA, 999 bp, Methylobacterium sp.
  source 1..999: mol_type = genomic DNA
atgttcatccgatctcacttgcggcccggtggccgccggctcgccggggcgcggctcgcgaccgcactgatgctggccacgatgaccctggccaccgtgacgctgggtccggtggccgcccgcgcggacgaggtcgtcctgcgggtcggcgaccagaagggcgggaaccggtcgctcctcgagatcgccggctacgcgaaggatctgccctaccggatcgcgtggtcggaattccccgccgccgcgccgatcttggaggcgctcaacgccggcgccctcgatgtcggctacaccggcgatctctccttcctcaccgtcgttgcggccggggcaccgatcaaggcgatcggcggcaccaagtccgatccccggacgcagacgatcctggtccgcgcggattcgccgatccgctcggccgcggatctgaagggcaagcggctcgccggcacccgcggcggctggggccagttcctgatcagcgcgacgctggaaaaggccgggatcgcgccctccgaggctaccttcgcgccgctcaacccggtcgacgccaaggtcgcgctgatggccggctcggtggatgcttgggcggtctgggagccctacgtcgcgttcgcgacgctcaaggacgaggcccggccgatcgcggacggcgctggcctcacgccgaccatcaccttcatcgtcgcatcggacggcgccatcgccaccaaacgggcggcgctgcaggacttcctgagccgcctgaaccgggcgcggctctggtcgctggaccacctcgacgcctacgcccggaacacggccgcgctgaccaagatgccggaggacgtcctgcgcgcggcctacacggcgcagcggaccagcccgatcgcgctcgacgagggcgtcgtgaaggagatgcaagacgcctcggaccgggcgacgcggtacggcatcctgtcgaagacgctcgacgtcggccgggccttggaccggagcttcacggaagcggcctcgaactga

SEQ ID NO: 71 - DNA, 3090 bp, Methylobacterium sp.
  source 1..3090: mol_type = genomic DNA
atggccaaagccaaagcccgcaagcctgagaccgcagccaagtccaagggaagggccgaggaacggaccgacgcacagctctcggccctgttcgacgcggcggccccggccgcgcgcgacacccgcgtcatctcggtgcgcggcgcccgcgagcacaatctcaagaacgtcgatctgacgattccccgcgaccggttcgtggtgttcaccggcctgtcgggttcgggcaagtcgtcgctcgccttcgacacgatctacgcggagggccagcgccgctacgtcgaatcgctctcggcctatgcccgccagttcctggagatgatgagcaagcccgacgtcgatcagatcgacgggctctcgccggccatctccatcgagcagaagaccacctcgaagaacccgcgctccaccgtcggcacggtcaccgaaatctacgattacatgcgcctgctctgggcgcgggtcggcatcccctactcgcccgccaccggcgagccgatcgagagccagaccgtctcccagatggtcgatcgggtgctggaactgccggagaagacccggctgtatctcctcgctcccgtggtccgcgggcggaagggcgagtatcgcaaggagatcgccgagttccagaagaagggcttccagcgccttcgcatcgacggcgagtactacgcgatcgacgacgtgccgaagctcgacaagaagctcaagcacgacatcgacgtggtggtggaccgcatcgtcgtgcgcgacgacatcgccgcgcggctggccgactccttcgagaccgcgctcgaactcgccgacggtatcgccgacatcgagttcgccgacgcgccggagggcgaggcgccgaagaagatcacgttctcgtcgcgcttcgcctgcccggtctccggcttcaccatccccgagatcgagccgcggctgttctcgttcaacaacccgttcggcgcctgcccgacctgcggcggcatcggccacgagatgcggatcgaccccgagctggtgatctccgattccgcgctgaccctgaagcgcggcgcggtgggcccctgggcgaaatcgacctcaccctattacgaccagacgctcgacgccctggccaagcatttcggcttcaagacctccgtggcgtggtcggcgctgccggaacaggcccgcgaggtgatcctgttcggcacgggcaaggagtcggtgcgcttcgactacaacgacggcctgcgctcctactcggtcaacaagccgttcgagggcgtgatcccgaacctggagcggcgctacaaggagaccgagagcgacgcctctcgtgaagaaatcggccgcttcatgagcgccaccccttgcgccgcctgcgacggcaaaaggctcaagcccgaggcgcttgcggtcaagatcgaccggcaggacatcggccaagtcaccgccctgtcggtgcgcgaggcgcatcgctggttctcggagatctcgggcaagctgacggacaagcagaacgagatcgcggtccgcatcctcaaggagatccgcgaccgcctgaccttcctcgtcgatgtcggcctcgaatacctgacgctcgcccgcggctcgggctcgctctcgggcggcgagagccagcgcatccggttggcctcgcagatcggttcgggcctgaccggcgtgctctacgtgctcgatgagccgtcgatcggcctgcaccagcgcgacaacgagcggctgctcggcacgctcaagcgcctgcgggatctgggcaactcggtcatcgtggtcgagcacgacgaggacgcgatcctccaggccgactacgtggtcgatgtcggcccgggcgccggcatccatggcggcgagatcgtcgcgcagggcacgccggaggagcttctcaaggatccggcttcgctgaccgcgaagtacctcaccggcgaactctcggtgcggacgccgaaagcccggcgcaagcccggacgcgggatgctgcggctggtcggcgcgcgcggccacaacctgaagaacgtgacggcggagatcccgctcggcaccttcacctgcatcagcggtgtctccggcggcggcaagtccacgctgatcatcgacacgctctacaaggcggcggccaagcgcctcaacggcgccctggagcacccggccccgttcgagcggatcgagggattggagcatctcgacaaggtcatcgacatcgaccagtcgccgatcggccgcaccccgcgctcgaacccggcgacctataccggcgccttcaccccgatccgcgactggttcgccggcctgcccgaggccaaggcccgcggctaccaggccgggcgcttctcgttcaacgtgaagggcgggcgctgcgaggcctgctcgggcgacggcgtcatcaagatcgagatgcacttcctgcccgacgtctacgtcacctgcgacgtgtgcaagggcaagcgctacgaccgcgagacgctggaggtgcgctaccgcaacaagtccatcgccgacgtactcgacatgaccgtcgaggaggccgccgacctgttcaaggccgtgccgtcgatccgcgaaaagatggagacgctggcccgcgtcggcctgcactacgtccgcgtcggccagcaggcgaccacgctttcgggcggcgaggcgcaaagggtgaagctctccaaggagctgtcgaagcgcgccaccggccgcaccctctacatcctcgacgagccgaccaccggcctgcacttccacgatgtcgccaagctgatggaggtgctccacgagctggtcgatcagggcaacaccgtcgtggtgatcgagcacaatctggaggtcatcaagaccgccgattgggtgatcgacatgggccccgagggcggcgacggcggcggccgcgtcgtggcgcagggcacgcctgaggagatcgccgcgagcacggcgagccataccgggcgcttcctgcgcgaggtgctggcgcgccgcccggccgggaaggccgtcaaggacgcggccaaagactccggcaaggacgcgggcaaggatgcggccaaggcgaaggcgggcgccgcatcgggccgccgcagcaacgccgcgggccggcaggctgccgaatag

SEQ ID NO: 72 - DNA, 681 bp, Methylobacterium sp.
  source 1..681: mol_type = genomic DNA
gtggtcgacgaggcgggcaagccggtcacgtcggcctgcacggcgccggccttcgagacccacggactcgccggctcgatcagcgtcgtcgaccgcacctatcactacgtctacaccgacgtgctgcccgaggattgcggcctcgcccccgagaagcgccgcaccgggctgttcctgcgcaccgcgcaggacctgtcggggccgaaggtctggtcgacggcgcgcaagctcgccgggccgctgccgcccgggaccctcgtgcgggtggcccgcgccaagggcatgcagcgctgggcggtctcctacacgtgccagcggcccgccaacgccccgggcggtccggtggcggatatctgcctgcaatacacggccgacatgaacctcgacgggatcggcgccctcaagctctacgccgatccggtggaggcgggccgctccgccgcctatctcgggctgcgctcggggggcgacggcagcggccggtacgaccgcagcgcgcatttctggatgaccgacgcggagggcaacctcgacacgccggcgatctacccgaacaaggcgggcttcctgacctggctcgaccggctcgccccgaccgcctccgggcgcgacgcgtcgagcctgtacgggcggccggtctactgggcgacgtggacggtgcgcccgatcggcgcgcaataa

SEQ ID NO: 73 - DNA, 210 bp, Methylobacterium sp.
  source 1..210: mol_type = genomic DNA
gtgccgccgggcaccccctacacgatcgagatctgggaccggatcggcggcaagctcgagttgcacctcgccacgatctacccgctgaccgcggcgatcgccgcattcgaggccgcctgcctggaatggccgacgaacgaggtcaccctgcgggatcgggctcggatcgtgcggaagcgggagctgccgccgcggagcgccaccgggtga

SEQ ID NO: 74 - DNA, 639 bp, Methylobacterium sp.
  source 1..639: mol_type = genomic DNA
gtgattcacgatcgcaaagccccaatcacgcgacgtctcgtgctggtccggcatggccagagcgtagccaaccgatccggtctgttcacgggattgctggactcgcccttgaccgagcagggtcggatagaagccgtggcagccgggcggcgtttggccgagcgcagctggcgcttttccgatgccttcacctcgacgctgacgcgggccgtcgtgagcggccggcttatcctcgatacgctcgggcaacccggattaatccctcaacgcttcgccgcgctcgacgagcgagactacggcgacctcagcgggctcgacaagaccgccgccgatgcgcgctggggggcggagcggatcgagacctggcggcgctcctacgccgaggcgccgccgaacggtgagagcctgcgcgataccgtcgcccgcatcgtgccatgctacctccgatccatcctaccggcggtcatgggcggggacgtgctcgtcgtcgcccacggtaattgcctgcgggcgctcgttatggcgctcgacgacctcagcccggcggaggtcgagcacctcgaactcgcgacgggctccgtcaggatctatgagtttgctgcggacacgacgatcgaggcccgctggatcgacggttga

SEQ ID NO: 75 - DNA, 397 bp, Methylobacterium sp.
  source 1..397: mol_type = genomic DNA
gcgggcattgtccgcttggtccgggcgtcgccgcgacggcgacctgcgggcgtggttgttcacgatcgagcgcaacctgttcctggccgccgtccggcgccggggccggcgcggcgccgatctcggcgcggaagcgttggagcaggtgccggatccgagcgccgacccgggggcagctttgggagcccgcgacgtgctcgccgggctcgacaccctgcccgaggagcagcgctcggtgctgctcctcgtggcggtggaggacctgtcctatgccgaggcggcacaggtgctcggggtgccgctcggcacggtgatgtcgcgattgagccgggcgcgcacgcggatgcgcggatttctggagacgggccggacaggcctgctgaggagggttaaatga

SEQ ID NO: 76 - DNA, 657 bp, Methylobacterium sp.
  source 1..657: mol_type = genomic DNA
atggtgacatccccgatctggcacgtcgtatcggctctggccgtggccgggacactggcggttcatggcggacctgcgcacgcgacgccgcggcatgcgcctgccggcacgctgcacttcgatgatggcggcgaacgctaccgcggctaccgcatcgagatggcccgggacgtcccgaacgccgagatcggccagttgcgtcaggcggccgagcatcaggtcgacatcgtggaggcgacgagcctggacgagggcaccaaagccttcctgcgccgcttcccggtcgtcgtccattccggggccggtgagcgcagccattacagcggcggcgaccacgtcgacatcgcggtcgaggacccgaaggacgcccgccccatcctgctgcacgagtccatgcacgtctaccacttccggaggctgccgggcggccggaataatccggacatcctgacgttctacgggcgagcgaaggacgggggcttctacccggcaggcgcctacctcctgacgaaccagggagagttcttcgccatgaccgccagcgtctacctgcacggcaagctggcgcgcgaacccttcacccgcgacgaactccggcagaagcaaccggtctactaccgcttcctcacccggctgttcggcccggtcggaacctaa

SEQ ID NO: 77 - DNA, 318 bp, Methylobacterium sp.
  source 1..318: mol_type = genomic DNA
ttggctgaaaccctgcccctgcgccacggcgcctatgtcggcgtgggcacggattgcacgaacccgcggaacgtcgagttgcgaacgtacgacggaggcggtctggggagttcgaaggcgaatgattgccggagccgggttctgcgacagcagggaaacgtgttcgaaattgagcaggattgccgccaattcggcggcccgaaggtcgagggtagcaccgaacgctcccccatccgggtcgacgggccggaacgctacacggacatgacggacggcggcaacgagagctttcgcttatgccccggactgaaaccgtga

SEQ ID NO: 78 - DNA, 6321 bp, Methylobacterium sp.
  source 1..6321: mol_type = genomic DNA
gtgcgcggcggtctgctcgccgagggagcggccgcggccgtcgagggtctcggcgaggcgcgaggcctcgctgagcgccccgtcggcggcgccacgcagggcctcgacctgggccgagacgccggcgctggcgcgaccggtctcggcggcgatgcgctccagggccccctggacctgctcgatccggccggcgagaccccgctcgatcgcgccgaggttggtgtcggccccggccacgaggtcggtgagcgcggtgttggcggcctcgatccgctgcagcaggctgccgagttcgcgacccatccggtcgttggtctcgaacagctccgagagggtgcggcccgcgccgtcctctacggcggcgcgcagcagctcggccgaggcccgcgactgcacggccagttcctcggtccgcgcccgcaggctctcggccaggggcacgccggtctcggcgagcgcccgctcgatcgcctcggcccggtccgccagggcccgggacaggtccatcatcggcccgtcgatggcgccgcgcaggcgctcgacatggccctccaggctgtcgccgaccgcgcggccgctgccctcgacgctggcgacgagcgcgccgccgcgctcctcgatggcgcgcaccagggtctcggcgctctcggcgaggcggcgggacagggcctcgccgcgggcatccaccagggcgccgagcgccgtggcacggcgctcgaacccggccccgagggcctcggtgcgggcgtcgatcaggccggcgagcgcggcctcgcggcggctgagggtctcggccagcgccgcgttgtgggcgtcgacctgctcggcatgggcccgcgcccgctcgtcgagggcggcgaccagggacgccgtgcgctcgtccatcaggcccgcgaaggcctcgtggcggtcgtcgaaggcggtggccagggtgtcggcgcgctgcgccaccagggtcgcgaacagccgcatgcggctgtcgatgcgctgggtgaaggcatcggcgcgggcgtcgacgccacgggccagggcctcgctctgcgcctccacggcctgcgccagggccgcggtgcggccctcgacgatgccgtcgagatcccgcagccgggtgtcgaaggcctcgaccagggcgccggtacgaccctcgacgacgccgtcgaaatcgcgcagccgggtgtcgaaggtctcgaccagagcgccggtacggcctcggacgacgccgtcgaggtcgcgcagccgggcgtcgaaggtctgaaccagggcgccggtgcgaccttcgacgacgccgtcgagatcccgcagccgggtgtcgaagacctccacgagggcgccggtacggccctcggccgccgccaccagggcctggctgcggtcctccagaagcccctcgatcgacgctcgggcctgctcgaaggtcgaccgcagggtctcgatgcgcgcgtcgacgcgctcggacacctcgcggccggtgccctcgaccagcacgccgaggcggttctgcacccgatcgaacagggtctgcagctcgcccgtccgctccttgagggtctcggcgagccggttctcgcgctcctcgagcaggccggcgatggccgggttcaccccctcgatggtcgcctggagcgcctgggtgcggtgctcgatggtcccggcgatctggcggccgcggttgtccacgaggcggtcgatctcgccctgcgcccgctcgaacaggccctgaaggtccgagaggcgggaatccagcaggtccgccatctgggcgggtgccgcgtcgagggtcgcgcgcaggccctcggtccgggccgcgatggcgtcggcgacgtcctggccgcgtccgtccacgaggtcgcgcaggccggcctgagcctcctcgaagagggtctgcaggcgctgcgtgcgcgcgtcgagggtggcggcgagctggcgctcgcgggtctcgaccagcgccgagacggcggcgggcgcctcctccagcaccgcgcgcagggattggatgcgggcatcgaccgtggcggcgaagcggccgccgccgccctcgaccacgccgtcgagggccgcctgggcctcctcgaacagggtgcgcagggcctgggcccgggcggtgagggtgtcggcgaggcggttgccgcggccgtcgaccaggctctcgagatggccgagccgctcgtcgaacaggctgccgagctggcgggtccgttcgtccagggcgccgctggcggagccgaacgcgccctccagggcggtgaccagcgcctgcccgcggccgtccagcgcggcgtggatcgggcccatgcggccgtcgagggtggcggccagggtgccggcgtggtcgtccagcatgacgcggatctggttggtccggtcgtccagcagggtgtggaccgagcgcacgccctcgtcgagcaggccgctgatgtcggcggtgcggctcgacagggtgtggcccaggcggccctcgccggcatcgagcatccgctcggcatccgcgatggtctgctggagctttgcgagcacgctgtcgccgcgctggtcgatcagctcggcgagggaggtgccgccgcggtcgagcagacgggccagctcggtgatccggccgccgatcgccccgaacacggcgcggcccttctcgtcgagctcgcccgacatggcggcgatgcgctcgtcgatgagacggccgaggaggtcggcgcgctcggcgaaggcggcgtgcacggcctcggtgcgggcggtcaaagccgcgtcggcgctctcggtgcgctcgtccaccagccggaccatcgactccgcacggttcgccagcgcggactcgaggccgcgggtggcctcctcgatggtgccggtgagggcgcggacgcgctcggccatggcgccgtcgaggccgccggtccgatcggcgatcatcgccacgaggtcgcgggtccggccggccagctcctcgtccaccgcgcgggtgcgcgcctcgatgaggcggatcgcctcgaaggcctgcacgccgaagctctcgtccacgaggcgggcgcgctcgtccagggtctcggcgatcgcctgcaggcggccgatgacgccggtgtcgaaatggccggcgctctcctcgatccggcgagcgatctcgcccgtgtgccgggcgatgtcctggccgaggccgttggtgcggccctccagcatctccatcaggccgcgatggccctcggccatgacccgcgccatctcgtcggtccgggcgcgcagcgcctcgtccgccgcgctggtgcggctgccgagatgaccgtcgatctggccgacgaggtcggccacggtggcggcgatcgcggcggcccgctccgcggagcgcgtctccaggccccccagatgggtctcgatcaggctgccggcctcgcgggcccgggccgtgatcgtctcggccacggcctcgcccttgccggtgaggagccggtccatctcctccagacgggcgccgacgctctcgccgagggcggcggtgtcgcggccgatccggtcggccaggatgtcgccgcgggcgatggtctcctgcagggcggcgaggcggctgcccagcgtctcgtcgatcgcccggacccggccgtcggcggtctcggcgaagctcgcaccggtctggctgagggtgtcggcgatgcgggcgccctggctcgccacggcgagcacgatgtcgcggccggtgccgctgatccgggcctgggtctcgtcggcatggctggcgagcagctcggcgacgccgcggccatgcgcgccgaaggccgcttccatctcgccgatccgggcggcgaggtcggccgagatcgcgcgggcggcctcggcgaggctgccggtggcgtcgccggtccgggcggcgagttcgcggttgaggtcgtcgagggcggcgcggacggccgagacggcggccgtgccgcgctcgccgagttccgtgccggcgcggtcggcggcgtcgcggaaccgggcggtgaggatcgcagtctcggcctccacggcgccggtgacctcgccggaggcggtgcgcagggtctccagggccgcggtgagccgggcggcggcatcccgcgtctgcgtctcggcggcctggccggcctgatcggcggcggcgcgcagcgccgcggcggcctcctcggtgcgcgccgacagggtggtggcggaatccgcgacccggcgctcgaacacccgcacggcctccagggtccgggcctcgaccgcgccggtggcctcgtcggtcgtgccgcgcagggcgctggtggccgcctcggccccgcgctggagcgccgcgaccgcctcggcggtgcggccgtcgatctcgcccgacaggctctcgacggtgccccgcagggattggaccgccgaggaggcccccgcctcgaagccctggcccagggccgcgaaggcggcctccaggcgcgccgccaccgtctcggtccggtgcgcgatgctgccgccggcctgctcggccgcggctgcgacccggtgctccaggtccgcggcccgaaccgtcacggtctcgtcgaggcgcgccgcgatgccgtccatccgctcggcgagggcgcttccgcgctccgacagcccttcggcgatccgctcggcggtgccgacgaggtcgtcgcgcacggcgccggcacgggccgtcagggtctcgccggcgccgctggccgccagcgccagggcagcctgggcatcgcgcccgcggccctcgatcgtctcggcgaccgacaggccggcgctctcgatctcggcgcgcagcgcgaggccgcggccggcgatgtcctgggtgagggccgagccggtctcggcgaagcgggcggtgatgtcggcgcccatcgcgccgaagcgctcggtcagctccgaaccccgggacaggaagacggattccagccccgtcacggaccgctcgaagctctcgcgcacggtgccggcctggcgctccagcgcgtcgctgacgcggctgccggtctcctccagggtccgggtgagggcgccggcattctcggacaggcgatcggccagggtggtgccggcggcctggaaggcgccggtcgccgcgccggcgcgatcctcgaagctgcgggtcagggtctcgccgacctcctggatggcgccgcgcacctcctggctggtggccgcgagggtgtcgacgaggtcgctgccgcgggcggtcatgaggttgaccacccggtcgccggcctcgcccagggaggcggtgatcgcggcgccgcgggtctccagggcgccggtcacccgctcgcccgcgccggtcacggccgacacgatccggtccgaggcgccttccaggtcctggctcaggctctggtgggagccggcaatggcgctgcgcacccggtcggcgttggtgacgatcgcctcgcgctgggccaccagctcgtccacgagggagcggatgcggatctcgttgtcggaataggcccgctccagggtggcgatctcgccccggaccagggtctccagctcgccggcccgggcgagcgcccgctcgacgccgtcgcccaccgccgccacctcgcggcgcacggtctgcgacatggtcaggaccgcgtcggtggagaagctctcgggctcggccaaacgcaccgcgacctcgcccaccgcacgggcgacgaggcgcatctcctgggcgcgcaccgccagcatggcggcgatcacgaacagcaccaccgggccgaacagcgccgcggcgccgacgctcgcctgcagggcggagagccccgacacgacggcgcgcaggtcgccctcggacttcgaccacgcgatcagcaccagggcgccgatccagacggcgccggcggcgagcgcgagcaggtagggcttgggcgaggagcgcacccgcagggtctgctgcaggatgccgatgttgcgccggtcgtcgttggccaccagcgagcggtcggggggcaggagcccgccctcgcggcgcgggcggtccaggcccggcggcggcaggtcggaggcgagcggcgggtcgaggtcgaggcgcggcggcatggtccgcccggcggcatcgaggccgctgtcgagcagcggctcggcgtcacccacatcgggcaggcgcggctcggcgcggccatcggtgccgttcggcgtcagggcgtcgaggttcagcgcctgctcgatcgcagagagcgcggcctcggccgggtccttcagcttcttctcggtcgccattcaacgcctcgttacacgggttcaccgacgccttggggaaggctgatgccggagacgcaacgccccgtttaccattacaagactaaactcaggcagcgactgccgatacccgagccctcgactggccccagaaaacgttaa

SEQ ID NO: 79 - DNA, 1155 bp, Methylobacterium sp.
  source 1..1155: mol_type = genomic DNA
atgctggcgatgaactaccgcggaccgtaccgcgtgcgcgtcgatcgcaagccgatcccggtgtgcgagcatgttgaggatgcggtcatccgagttacgcggacctgtgtctgcggctctgatcttcacctgtaccatggaatggtgccggatacgcgcgtcggcacgacgttcggccacgaattcacgggcgttgttgaggaggtcggctccggcgtgcgcaatctcgccgtcggcgaccacgtgctggtcccgttcaacatcgcctgcggccgctgtcacttctgcaagcaggggctgttcgggaactgccacgaggcgaacgcgcaggcgaccgcggtcggcggcatctacggatactcgcacaccgccggcggctacgacggcggccaggccgagtacgtccgggtgccctacgccgatgtcggcccgtgcaagatccccgagacgatggacctcgacgacgcggtgctgctcaccgacgtcgtgccgaccggctaccaggcggccgagatgggcgggatccagcgcggcgacaccgtcgtggtcttcggcgccggaccggtcggcatcatggcggcgcgctgcgcgtggctgttcggggccgggcgggtgatcgtcatcgaccacgtcgagtaccggctcgacttcgcgcggcggtactgtcccgccgaggtctacaatttccgctcgatcggggatcccgtggtcttcctgaagaagaccaccgactccctcggggccgacgtctgcatcgacgcggtgggcggcgacgcggcggggagcgccctgcacaccctgatggggaccaagctgaagctcgagggcggctcggcggtggcgctccactgggcgatcaactcggtcaagaagggcgggatcgtctcgatcgtcggggtctacgggccgaccggcaacctgatcccgatcggcaacgtcctgaacaagggcatcaccatccgggccaaccaggcctcggtgaagcgcctgctgccgaagctgattgcccatatcgaggcgggccgcctgaaccccaaggacctgatcacccacaaggtgcccctcgaggaggccgcggatgcctaccacctcttctcggccaagctcgacggctgcatcaagccggtcctggtccccccgacggcacgcgcgtga

SEQ ID NO: 80 - DNA, 495 bp, Methylobacterium sp.
  source 1..495: mol_type = genomic DNA
atgagaaaccgtccgatcgatccgtcgacggtcccgggctggggcgtcgacgcggatccgcagaacgatccgacctacccgatgcgcgacatcgcgcgcgacgacagccggggcatgaactggacgcggccgccgcagcagcgggcgcgcgtcgaggtcctcacctcgatcgagtacaacgcccggccggcggtcgtcggcacgtcgaccccgccgcgcggcgtgagcggcgtcatccgccggcaggcgttccgctacagcgagtcccagtgggcgcactggctgatgctgatggccgccgaccgggtgaacgtggtcgagggcgtggtcgacgacctcgcgcggggccgcgtccccaacgtcccggccgagatgggcgcccgggccgagctggcccacaaccggtccgggctggcggggaagctcgcgctgaccggcgcggcgatcgggctcgggatcctggtctcgcgcctcgcccgcgcggagcgcctctag

SEQ ID NO: 81 - DNA, 903 bp, Methylobacterium sp.
  source 1..903: mol_type = genomic DNA
atgccgctcctcgccctgccctttccggccatcgatccggtggcgatcgcgatcgggccgatcacgatcaaatggtacgcgctcgcctacatcgccggcctgatcggcggctggtactacgcgcgccgcctcgtcatggccgacagcctctggggcgtggtgaagcgcccgcaggtcgtcgacatcgacgacctcgtcgtctgggtggcgctcggcgtcgtgctcggcgggcggatcggctacgtgctgttctacaatctgccgatgtacatcgccgatccgtgggagatcctggcgatccgcaacggcggcatgtcgttccacggcggcttcatcggggcgatcctggccttcgtgctgttcgcccgcggcaagggtctcaacgcctacacgctcctcgatatcggcgccgtggtggtgccgatcggcctgttcttcggccggatcgccaatttcgtgaatggcgagctctggggccgcgtggcgccggacttccgctacgcgatcgtgtttccgagcggcgggccgctgccgcgccacccgagccaactctacgaggcggccaccgagggcctgctgctgttcatcgtgatggcggtcagcgtccgccggttcggcttccgcaagcccgggctcctcggcggcatcttcgtcctcggctacgcgctggcccggacattctgcgagttcttccgcgagccggatcgccagctcggcttcctgttcggcgaccatctcggcccgatgggcggcggcgtgaccatgggcatgctgctgtgcgtgccgatgatgatcgtcggcctcacctacatcgtcctggccgcgaccggccggacacggccccgccatccggtggaagcgccggccgccgaggccgcccgcaaggcggccgtcgaggcgtga

SEQ ID NO: 82 - DNA, 780 bp, Methylobacterium sp.
  source 1..780: mol_type = genomic DNA
gtgaccgcaggcatgttcatcgaggcgccggaactctccgcccatgcgggcgtgcgccacgccttcttcacccgcgtcggcggggtctctgagggcctgtacgcgtcgctcaacggcggtctcggctcccaggacgcgccggagcgcgtcgtcgagaaccgggcgcggatgtgcgcgcagctcggcttggcgcaggaccggctggtgagcctgtatcaggtccattcggccgatgtggtcaccgtcgaggctcccttcgcggcggagcggccgaaggccgacgccatggtgacgcgggttccgggcctggcgctcggcatcgccaccgcggattgcgggccgatcctgttcgcggatcccgagaacggagtcgtcggcgccgcccatgccggctggaagggtgcgctcaccggcgtgatcggcgcgaccgtgtcggcgatggaggcgctcggggcgcggcgcagccgcatcgtcgccgtcctgggaccgacgatcgggcaggcctcctacgaggtcggtccggacttcgtcgcgcgcttccggagcgatgcccccggcatggagcgcttcctcggccccggtacgcggcccgggcacgcccagttcgatcttccgggcttcatcctggcccggctcggcgaggccgagatcggcgaggccaccgcactcaatctctgcacctccgccgatccggagcggttctacagctaccggcggaccacgcatcgaggcgaggcggattacggccgcctgatctccgcgatcacgctggtgccctga

SEQ ID NO: 83 - DNA, 1071 bp, Methylobacterium sp.
  source 1..1071: mol_type = genomic DNA
gtgaccccgctcggaaccgagatcgcggcgctgatccggcagaacgggccgatcggcgtcgaccgctacatggccctctgcctcggccacccggtccacggctactaccgcacccgcgatccgctgggcgcccagggcgacttcaccaccgcgcccgagatcagccagatgttcggcgagctgctcggggcctggacagcctacgtccgcggctcgatcgggtcgccggatccgctcctcctggtggagctgggtcccggccgcggcacgctgatggcggacgcgctgcgcgcgttgcgggcggcgctttcgggggtgcgcgtggcaccgcacctcgtcgagacgagcccggtgctgcgggccgcgcaggcgcacgccctctccgggaccggcgccgcgtggcacgacagcatcgagacgctgcccgaggggccggcgatcatcctggccaacgagttcttcgattgcctgccggtgcgccagttcgagcgccggccgtcgggctggcacgagcggcaggtcggcctcgatccggcgggcggcctcgccttcggcctgtcgccggaaccggttccgggcctcgcggcggacggcccggacggcgcgctgatgagcgtgccggcggccgggctcgcgctgatccgcgcgctggcgcggcggctgtgttccgaaggcggcgccctgctggccatcgattacggccatgtgcgtccgggcttcggcgacaccctccaggcgctggcgggacatcgcttcgccgacccgctggcggagcccggcgaggcggacctgacgcatcacgtcgatttcgccgccctcgcccaggcggcgcgggccgagggcgccgcgatccacggccccgtcgatcagggcgacttcctggccgctctcggactcggggcccgtgccgagcgtctgagggcacgggcgagcccggcgcaggccgccgcgatcgacgcggcggtcgcgcgcctgaccgatccggcccggggcgggatgggaagcctgttcaaggtgctggcggtgagcggcccgtccgtcgggccgctcccgggatttccgggggcctga

SEQ ID NO: 84 - DNA, 1140 bp, Methylobacterium sp.
  source 1..1140: mol_type = genomic DNA
atggggccgggtcgcgaagcggtggacgcggcagcccttctcgatgggttcagtgcgtgcctctcggggctcggcctgccgctggcccgcgccaccacccacgcgccgaccctgcatccgtcgttccggtgggtgatgcgcgtctggcatccaggaacgtcgagcctagccctgcgtcgccgccacggcatcgagggcacgccgaccttccacggcaacaccgtcgagcacgtggtcgagacgcggaccccactccaatgccgtctcgacggggatgggccgctgccgtttcccgtgcttggcgagcttcgcaacgagggtctgaccgactacctcatcgcgccgctgcgcgccgcgcgtggacgcatgggtgccgcctcctgggccacggcgagaccgggcggcttcacgccgatcgagatcgagacgttgctggcgctggtcgagccgttctcgctgctgttcgaaatcaaggcgctcgacgacatgcttggcgcggtcctgagcgcctatgtcgggcgcgacccagcgcggcaaatcctggccggcacagtgcggcgcggcgacgtacgcctcatgcgggccgcgatgatgctgaccgatctgcgcgggttcggtgaactgtccgaccggcaaagccccgaccatgtcgtggccgccctcaaccggatgttcgatgccatcgtgccggccgtcgaggcggaaggcggcgaggtgctaaagtatattggtgacgggctgctggccgtgttcgatgccgatcgcgacgaggccgaggcgcgccgtgccgccttgcgtgccgccgaggccgcactcgatgcgcttgcgaccctgcgcgacggtgacagggctgcctttgaggtcggcgtcgcacttcatgttggggaggtcgcctacggcaatatcggcggcggcgaccgggtcgacttcacggcgatcggccgcgatctcaacgtgctcgctcgcgtcgagcgcctgtgcaagacctacgacacgccgctgatcgccaccgacaccttcctccacggcctggcgcatgccctcgaaccgctcggcattgtcgcccttcgcggtttcgccgagcgtcacgccttgttcgggtgtcgcaggaccgcgcctgtcgaagcaccggcggtctga

SEQ ID NO: 85 - DNA, 984 bp, Methylobacterium sp.
  source 1..984: mol_type = genomic DNA
atgacccatgtccgttccagcctgcgcgccctcctcgccggagctgccttgctcctcgcccagggccagcccggatccgccgccggtttcgacggcgcgatcaagaacaacgcgctggccctgaacgctgccggcacagtcgccgcggtgtcgaacagcgaggagagcgccgtcatcgtctacgatgtcgccaaggggacggtgctgcgccgcctcgacggcttcgtgacgccgcgcaacatcgtgttcgcaccggacggcacccgcttctacgtctccgacagcggcaccggccggatcacgatctacgagaccgccacaggcaaggaggtcggcattctcgccgccgggccaggggccttcggcaccgtgctctcggccgacgggggcaagctctacgtcaacaacgaggccgcgagcacgctgaccgtgttcgacaccaagaccatgctcgcggaggccgtgatccccggcttcgcgcagccgcgccagggcgtgaagctgtcgcccgacggcaagaccgtgttcgtgacgaacttcctcggcgacaagatcacgttggtcgacaccgccacgaacaagatcaccggcgagatcgccgggttcgacaagctgcgcgccatctcgatcaccaaggacggcaagacgctgtttgccgccaacagcggccgcaacaccgttggcgtggtcgatgtcgccgcccgcaaggtcacgtccgaagtcacggtgggcaaagacccctacggcgccgcgctgaccccggacgggcgcttcgtctattcgggcaacctcaaggacaattcgctctcggtgatcgacaccggtacactcaccgtcgtcgccacggtgaccggcctgaacgagccgcgccaagccatcgcgttctcgaccgacaacgcccgcgcctacgtcctcaaccgcgatctcagcgtcgccgtggtcgatcgcgcgaagaacgcggtggtttcgacgatgaagccgtga

SEQ ID NO: 86 - DNA, 201 bp, Methylobacterium sp.
  source 1..201: mol_type = genomic DNA
gtgctggcggtctggccgccgaaggtgctgtcgctgacgctgatcgcctcattctccggcgccgtgtggcgggaccgggcgatccgggcgcgactcgcccatgccgagatcccgatgatgccgcgcctcctcaccgtcgggatcgcggccctgccgatcgcgatctcggtcctcgcggccaccgccctgctccgggcgtga

SEQ ID NO: 87 - DNA, 1146 bp, Methylobacterium sp.
  source 1..1146: mol_type = genomic DNA
atgagagtcgcgatcgtccattactggctcatcgggatgcgcggcggcgagaaggtcgtcgaggcgctctgcgacctctatccggaagccgacatcttcacgcacgcctacgccccgcaatccatgtcgccgaccatcagggcgcaccgggtcaggacctcgttcatcggccggttgccgttcgcgaccagccgatacaaatcctatctgccgctgatgccgatggccctcgagcagctcgatctgcggggatacgatctgatcatcagcagcgaatcgggtccggcgaaggggatcatcccaccatccgacgcgctccatatctgttactgtcactcaccgatgcggtacgtctggaacatgtaccacgattatcgcgagcggacggggctgctcacgcgcctgctgatgccgccggtggcccactacgtgcgcaactgggacgcggtctcggcgggccgggtgcacgagttcatcgccaactcggacacggtcgcacgacgcatcgagacctactaccggcgccaagccaaggtcatccacccgcccgtggatacggcggccttcgagatcgctccggatggccagcggggtgactaccatctgatggtcggcgaaatggtccgctacaagcggcccgaactggcgatccaggccttcaaccggttggagcagccgctcgtggtgatcggcggcggcgagatgctgcgtgagctgcgcagcatggccggtccgcacatcaagatcctcggtccgcagcccttcgaggtcttgaagcaccactacgcacggtgccaagccctgatcttccccggagaagaggatttcggcatcgtcccggtcgaggccatggcgagcggacggccggtcgtcgcgttcggcaagggcggtgtcaccgagacggtcatcgacggcgtgaccggcaccttcttccacgagcaatccgtcgacgcgctgatcgatgccgtccagcgctgccgggcgatcggggtggagccagagcgcctcgtccgccgcgcagccgacttcggtgtcggccgcttcgcggacgagatcagctgtttcgtcgacggggtcctcgcgcgcgagcgcttggcggcccctcgtcccccgcgtgagccgagccgcgcctatctggtccagtga

SEQ ID NO: 88 - DNA, 1536 bp, Methylobacterium sp.
  source 1..1536: mol_type = genomic DNA
gtggccagtcccgtcgccgtcaccgagatccagccgaaggcccaggcgcggcctgccccgcaggccgcgctcagccccggcgagatccgcgccatcgtgtacgggctgatgaccgcgatgctgctcgcggccctcgaccagaccatcgtggcgaccgccatgccgaccatcggcctcgacctcggcgacgccgcgaacctgccctggatcgtcaccgcctacctgctggcctccacggcggtgaccccgctctacggcaagctcagcgacatccacggccggcgggtcatgctgctgatcgccatcgcgaccttcgtggtcggctcgctcgcctgcgcgctcgcgcccacgatggtggcgctggcgcttgcccggggtctccagggtgtcggcggcggcggtctgatcgccctggcgcagaccatcctggccgacatcatgtcgcccaaggagcgggcgcgctaccaggtggtgatcgcgggcgtgttcgtgacggcctcggtggcggggccgctgctcggcggcctcttcgcccagcacctgcactggtcgctgatcttctggatcaacctgccgatcggcgtcctcgccttcgcgctcaccaacgccaacctcaagcgcctgccccgccacgagcgccgccaccggctggactatcccggcgccgccctgatggtggcgggctccgtcaccctcctgctggccctgagctggggcggggtgcgctatccctgggactcggcgccggtcctcgccctcctggccggggcggccgtgctaagcggcgccttcgcggcgcggctcgccacggcggccgagccgctgatccccacggaggtgctcaaggaccgggtggtctacagcgcgacactggccgcgtgcttcgccatgggcaccttcatcgggctcaccatctacgtgccgatcttcctcgaaggggtgatcgggctctcggcgagcgagtccggcgtggccctggtgccgctgatgatcggcaccgtcacgggcgcgaccctgtcgggccgctcgatgctgcatttccggcactacaagcgcctgccgctggcgatgatgtgcgtcagcctcgcctgctgcgccaccatcgcctggcagggccgggcgctgcccttctggctcatggaggtgctgttcgcgctgctctccatggggatcgggacgatcctgccgctctcgaccatctcgatccagaacgcggtggagacgcaccagctcggcatcgccaccgccgcgatgaacttcttccgctccctcggcggggcgctgatcgtggcggccttcggcacgatcgtgctcggcggcgccgccggcggggcggggggaggggcccacgacgtggagagcctgatccgcggcgcggacccggcccagctcgccctgaccttccgccacgtcttcctggccgcctgcctcggcctgctcggggccttcacgttcctggcgctgatggaggagcggcccctgcgcgagcgcacctcgccgaggatggccgccgagaccccggaggcctccgcctga

SEQ ID NO: 89 - DNA, 927 bp, Methylobacterium sp.
  source 1..927: mol_type = genomic DNA
atgaccgtcgcgatcctcctctccggccagggcggtcagcacccggcgatgttcaacctcacggctgatcatccggcggcccaggacgtgttcgcggccgccagaccgctcctcggcggcgccgacccgcgcgaccttgtccgcgcgggcggcaacaacctgcacgcgaataggaccggccagatcctgtgctgcgtcgccgccctggcggcgtggcgggccctggccgaagcggagccggaccgggcgatcgtggccggctacagcatcggcgatctcgcggcctggggcgtcgccgggcgcctcgaccccgcggacgtgctggccctggcggcccggcgggcggaggccatggacgcggcctccggctcggggttcgggctggcggggatccgcggcctgagcctcgacgcgctcggagatctcgcggcgcgccacggcagccacctcgccatccgcaacgcggccgacagcggcgtcgtcggcggttcgcgcgcggcgctggaggcactgtgccgggaggcgaccgcctcgggcgcgcagcgggccgtggtcctgccggtccacacgccgtcgcacacgccgctgctcgacgccgcgacggcagccttccgggacgccctcgccgccgtgtcgtcgctccgcccgccgccccgcgcgccgcggctgatcagcgggctggacgggagcacggtgttccgcgacgcggacggtcgggagaagctggcgctccagatctcgcgcacgatcgactgggccgcctgcctggaggcgtgccgcgagtacggcgcggaccgcgtgctcgaactcgggcctggccacgcgctcgccacgatggcccgggccgcgctcccggcggcgcgcgtccacgccgtcgaggaattccgctcgctcggcggcgtggcggactggctcgcgcgaccttag

SEQ ID NO: 90 - DNA, 2313 bp, Methylobacterium sp.
  source 1..2313: mol_type = genomic DNA
atgaaccccgcaatgagtggtcccgcgccggatcccggccgcctgaccgtcatcggcgtgcgccaccattccccggcctgcgccgggctcgtccggcgcacgatcgcggcgctgcggccggcctgcgtgctgatcgaggggccggtcgacttcaacccgcacctgcccgacctcgccctcggccacgacctgccggtggcgatcttctcgttccgggccgacgcggcgggcagcgccgcctcctacacgccgttctgcgccttctcgccggaatggcaggccctggaggcgggccgggcggtgggtgccgagacgctgttctgcgacctgccggcctgggacccggccttcgggcgccgcgccaaccgctacgccgacctgcatggagcccgcgccgaggcggccgagcgggcgctggccgccgccctcggcgtcgccgaccaggacgccctctgggacgtcctcgccgaggcggcgcccgaggcggagctgccggcccggctcgaccgctacttcgccctgctgcgcccgcccggcacggacgatctcgccgaggaggcccgcgagcgcttcatgggcgcctacgcggcccacgccctgcgggcggccggcgaccggccggtcgtcctcgtctgcggcggctggcacgcggacgccgtccggcgccacgccgcgcgcgcggacgggacccggcccgagccggcgccgccggagccggacctgcgcaccggcagctacgtggtgccctacgcctatccccggctcgaccggttctcgggctacgcggccggcatgcctgcgccgggctactacgagcgcgtggccgagtcgggactcgcccccgcggccgactgggcgatgacggcgatcaccgcggctctgcgcgaggccgggcaggtcgtctccaccgccgaccgcgtcgcctggcgggtccacgccgaggccctcgcccggctgcgcgcccatcccgcgatcctgcgcaccgacctgatcgacgcggccctcgcggccctggtgaaggacgccctcgaccgcccgcccgcctgggcggccggcggcgccgcgcccggccacccggcgctcgccgcgatgctgcgggcgctcaccggcccgcgcgagggacgcctcgcccccggcacccggcagccgcccctcgtggccgacgtcgccgagcgcctgcgcgccgccgacctggagccggggccggcccgccgcagcgtcgacctcgactgggccgagcccggcgaccgggcgcgggcgcacctgctccaccggctggtcctgctcgggttgccggggatcgcccgcgagggcccggaccgcgccgggcccggcctgccgcgggagcgcttcacgctcgcgcgccacccgcactggctcggcgccctgatcgaggcctcgctctggggcgggaccctggagatggccgcctccgcccggatcaccgcccgggtcgaggcggcgcccgattccctcggaatcctcaccggagccctgtccgacgcgctgttcgccggcctcaccctggagggcgaactgctcgcccgcctgagcgcgggcatcgcggcggcgcacgacgctaccgccctgggcgccgccggggccgggatcgtgcggctgtaccggttcggcgacgccttcgcgccggcgcggccggccctggcccgcctctgcgccgtcctggcggcccgggcgctcttcgtcgtcgaggccgtcccggacccccgcgctgggctcggcgtgatcccgctggtcctcgcctgtcgcgatctgttccgcgaggtcggcgcggagatcccgggcctcgatgacctgcgcgccccgttctctgccatgctcggccgccgcctcgccgatccggagacgccgccggccctcgcgggggcggcgctcggcttccgggtcgcctgcggcgcggccggatcggacccggaggccgccctggcctggctgcgccgcttcggcctgcccgcgacgctcggcgacttcctcgccggcctgttcgccctcgcccgcgaagagattgccgccgacgcgaccctcgcctccgtcgagggtctggtggccgcctggggcgacgacgacttcctgcgcgcgttgccgtccctgcggatggccttcgcgtggttcccgccccgggagcgcgagcgcatcgccgtcgcgatcctgcgccggagcggcctcggcgaggcccgggccgaggtcgaggccctggcctggatgcgccagcgcgcccgcccggccgatcaggccgaggccctggcccgcgaggcgcgggtcgccgcgcggctcgcccgccacgggttgacctga

SEQ ID NO: 91 - protein, 340 aa, Methylobacterium sp.
MLLGTLAAGVTRPAMAEPVVEDVRIVDFDWVDGARQRPVPVRLYWPNTSSLRRRIPLVVFSHGLGQSRTGYSYLGRHWSSHGIASLHLQHVGSDSSVWTGNPLALLDRIERAAQEREAIARARDLRFALDRLLVQDGGAFGDRIDPRRIVAAGHSYGANTTLIAAGARVVRDGHALQARDPRIAAGIVISAPPFYGERDLRAVLGAVEIPTLHVTATEDVIQLPGRTSPFSDRLAVYEAIATPRKSLAVFQGGSHSIFTDRPLTGGLNLNPQVKAATAAGTLAFLDLAFRGDPEPLRAWSSTWRPILAVAPAGYASASLAQPAIATRGKKRTRAQPVEPL

SEQ ID NO: 92 - protein, 431 aa, Methylobacterium sp.
MLTRVSLTGSLGLALLAATAFPALAQQDRGGGLAQAEWAQNYDSAATMRVQRSNTPILSPQTLAATEQMVERYRDIVARGGWQAVSGAERLRVGSKSPAVTALRQRLIVSGDLDPAAGSSPVYDSYVEAGVRRFQARHGLNQTGAMNVTTVQAMNVPADVRLRQLELNAVRLRSYSGNLGERYVIVNIPAALVETVDGDHVATRHAAGVGKIDRQSPIMNAKIQEVNFNPYWTVPASIIKKDLIPKMQKDPAYLTDNKIRIFNGDREIPPSQVNWHSDEATRYRFRQDPGVDLNSMGFVRINIPNPHGVYMHDTPAKGIFGDDFRFVSSGCVRVQNVREYVSWILQGTPNANPDTIEAIIQGGQRVDARPVAPIPVYWTYITAWSTPDGLVQFRDDIYKRDGAGAPTASMAAAAAGPRNFNPDAPDDEQTN

SEQ ID NO: 93 - protein, 411 aa, Methylobacterium sp.
MTEIDTDAFLADLYALREIGRFRTGVHRPTFSAADMESRRWLMAKLEECGLEASIDGIGNVLGRHRGPGPHLLVGSHIETQNEAGWLDGALGVVAGLALARAGLPVDVVAFADEEGHFSGGFLGSRSAIGDLTEAEIEAARNRTDDTPLRAALEAAGLAGLPRMRLDPARYRGFLELHIEQGTQLESTGLHLGVVSGIVAIWQFQIVFDGNQDHAGGTTMAERRDAGLSAVRLLAAIDREFPKLCGPRSTWTTGRITLDPGGYSIIPGRAEVAFQFRDVSIPVLERMEACLEALVRESNRRERCPATLTALSKAIPAPCDPDLMRALSEAAEQVCPGRWQVMPSGAGHDAQNIARILPAAMLFVPSIGGISHHWAEDTSDADLAFGVRTLGAAAARVLATRKAGDSFRSLR

SEQ ID NO: 94 - protein, 199 aa, Methylobacterium sp.
MRRPDQPALTAAAHGRVSMPFLPTLAVVAILGVASLYCPTSKPGLPGARVEAGVSAAPGPTDFAPIAAVAAPGRPPAVIAFAEQYPLDAVIARTGSLPARPAVAARANAHVAAAGRRACPGRRCPETPRSNTDPMAPARGAADEAEDALLPSQAMPFAASVVETLVPAAQAVGDAANLVRSSARAVQGTVALAVADCLR

SEQ ID NO: 95 - protein, 313 aa, Methylobacterium sp.
MSNSSEPTLLTADQDLVLRFWGVRGSTPVSGPQYAEFGGSTPCIEVRCGQRMFIVDAGSGIYNLGQGHRTDLPREVDLLFSHLHLDHTAGLPFFKPAVLDCDRVINTYCGNLGGESAGRTLDRLFAPPLFPVTLDRLCCTFRHHGFEAGQTLTFPDGTRVATILLNHPQGSVGYRFEHAGKRLCLISDIEHSDPWPDPNLAAFVADADLMVYDGMFTDGEYPTCRGWGHSTWQKGVELARTAGVKALGIIHLHPAHSDTALRDMEADMQAEMPTAFIARECQSLTVGAPRAVGGRPNGRPAVAREMRRRIKVA

SEQ ID NO: 96 - protein, 323 aa, Methylobacterium sp.
MTFPLRAGLAAALTLAAAPAFAHAVLGVKQASPNQTYRGVVQIGHGCDGKPTTGVTVTIPEGVIAAKPMPKPGWQLATTKGAYGRAYPTHHGSVSEGVKTMAWTGGSLPDDQYDEFVFQARLTDAVAPGGTVYFPVRQDCDGATVDWSQVPAAGQAARDLTSPAPGVRIVAAAAAAAPAAPASTSIKAGDLTIAQPWIRATPGGAKVAGGYLTVTNTGKEPDRLVSASIPLAPRGEVHQMAMDKGVAKMAPVEAGLVIKPGETVVLKPGGYHLMFMDLTGPVKAGDTLDGTLTFARAGTVPVRFAVGAIGASAPEAAGGHQHH

SEQ ID NO: 97 - protein, 59 aa, Methylobacterium sp.
MHAIGTRPEHAIPMDQIAPVTSSRTRKKPHPALVVASIALAVGLWFVIYAVSGRVIWFW

SEQ ID NO: 98 - protein, 413 aa, Methylobacterium sp.
VDFTLSQSQTHWLTRVRAFIADAILPAAAAVSAERAQSREPSPTMERLKEKARAEGLWNLFLPPSAEHDTDAYRGAGLTNLDYALCAEEMGRVGIASEVFNCAAPDTGNMEVLHRYGTAAQKDRWLKPLMAGEIRSAFLMTEPEVASSDATNISTSIRRDGDHYVVDGRKWWSTGVGDPRCKIAILMGKTDPEAPRHRQQSQILVPMDTPGVRVERLLPVFGYEDAPKGHGEVVLENVRVPAGNLILGEGRGFEIAQGRLGPGRIHHCMRTIGAAEVALEAMARRLVSRVAFGKRISEQSVWEQRVAEARIDIEMTRLLCLKAADMMDKVGNKGAKLEIAMIKVAAPRVALKVIDDAIQAHGGAGVSEDFGLAKMYAHIRTLRLADGPDEVHNRSIARLEFGRYTNRSEAERA

SEQ ID NO: 99 - protein, 712 aa, Methylobacterium sp.
MPRLFRAGRCAALLLSACATPAAAQPQSSAASVTLSELSVVGAAATDAPSGETRGSLSVPSVDRQRAALAGTVGSVAFVDAATLQDRYANTLRDVLKDVPGVYVQERYGQELRLSVRGSGIARAFHLRGIELLQDGIPLNLADGSGDFYQVDPLALRSVEVYKGGNALTFGATTLGGAVNVVTPTAYTALAPNILRVDGGSFGTIREQAQMSRIDGPLDVLVNATLTNSDGFRAHEAQATKNFNVNIGYRIAPDIETRFYLGTYLTDQKLPGTLTLGQSLTTPTLANPAAISGNQSRKVETERIANRTSFLLDVGKLDVDTWAIHKSLYHPIFQAIDQDGWTYGISPHWAGTFDLGGFRDDVVLGLRAFAGQNSALQFVNVRGQRGAQTLNSLQSASSVEAYGENRFWFLPDVALMAGAKAFSSNRTFSDKGGLPGNPRPRFADVTYEGLNPKVGLLWQPRPDIQVFGDVVRSRDVPDFSDLVQQNLLSTTFVPLRAQRAWTYEAGARGRFGPLAFDVTLYRSDLRDELINFATNPGLNIPAATFNTPRSVHQGVEAAVTLDLVRDLAGTGDGVSLTQIWTHNDFRFVGDPVFGNNRIAGIPADVLRTVLSYRHPSGFHVAPSLDWVPQGAFADHANTLRVPGYVLLGVEAGIDFADGVALFVDARNLTNARYVSDIAVVANAAATAGGAGALAAFYPGSGRSVFGGIRAAF

SEQ ID NO: 100 - protein, 796 aa, Methylobacterium sp.
MSETESGPGSVTEADYRNLAETLPQLAWIAEADGTIVWYNQRWYDYTGTSLDEMRGWGWRTVHHPDHVAAVTERYRAAITLGRSWEDTFPLRGRDGSYRWFLSKALPHRDESGRILRWYGTNTDITVRRAVEERLRHSEQRFRALVDASAAVIWNTDAAGELMPPQVRWSTYTGQTEEAYQGWGWLDAVHPDDRGHAADAWAACVEARATYEVEYRLRRYDGVWRAMEVRGVPVLAEDGSLREWVGTCVDVTERKEAEEAVERARQAAEAANRAKSQFIANMSHELRTPLSAVIGYSEMLGEELEDIGQAALLPDLRKIEAAARHLLSLINDVLDISKIEAGRMTASAETFTVADLLRDVSDSTGSLVEKKGNRFVLDAGAAGEAGLGSMHQDQTKIRQCLLNLIGNAAKFTERGTISLTVRRHRDAGADWLSFAVADTGIGLTEAQIDRLFERFVQADDSTTRQFGGTGLGLAITRAFCRTMGGDIGVTSTPGAGATFTIRLPATLRPEDAPPTEAEAHTPVEPHEEHETVLLVDDDPAARELLQRFLEREGFHVRSANDGRAGLTLARALKPRAILLDVEMPRMDGWAVLHAVRNDPDLAGTPVIMTSVVAEQGLGQALGATDYFVKPIDWDRLKGLMERYRPAAPNEARVLVVDDDADARERLRRSLGREGWTVDEAENGRIALERVSQARPSLILLDLMMPEMDGFGFLRALRSRPDGDVPVVVLTAKEVTAAEKESLNRQADRVIAKGSMSLAEIGRQLRVLYARSATEPVPGQLQGLLDRLAEKDAGEPR

SEQ ID NO: 101 - protein, 89 aa, Methylobacterium sp.
MMHVKMLAAAAAVVGGLGFASAGAAPLAPLAADTVTGAAPISTVAYGCGPGFAPGPYGRCRPIYRRPRFYGPRCFFRPTPFGPRRVCRY

SEQ ID NO: 102 - protein, 562 aa, Methylobacterium sp.
MPIETVETAGRRRGADLLVEVLRSEGVRYIFGNPGTTELPLIDALTEAPDIAYILALQEATAVAMADGYAQGARRPAFLNLHTAGGLGHAMGGLVNSQVSGTPLVVTAGQQDLRHALTDPLLMGDLVAIADPVMKWAREVTSPDQIPILLRRAFHDAGAAPSGPVFLSLPMDVMEALSAVPAGETSTIDQRAVAGSLDRLAEKLAAIAPGRLALIAGDEIDASDASAQMVALADLLAAPVYGSSWPAHIPFPTAHPLWAGNLPTRADAIADILGRYDAVFALGGKSLITVLYSEVSAVPPGVQVFQLSADVRDLGRTYATCLSTVGDIRASLDALLPLLAPRLADRADAFAGLRSGAVTARAERRAKLAAAADAAFEDPVIAPLVAAREVARAVGAETTIVDEAPATLTHLRTFLDSPSAHQYAAMRGGVLGWGMPAAVGFSLGLDRAPVVCVVGDGAALYSPQALWTAAHEKLPVTFVVINNAEYNILKTFMKGQAHYASVRANRFIAMDLTDPRIDFPALAASMGVPARRVTRAADIAPAIEAGIRSGGANLVEVVVRAT

SEQ ID NO: 103 - protein, 157 aa, Methylobacterium sp.
MNSAAIKQLNESMVLVHTGIARRATTTVAAQIAVTRARAIDKELSELYRLVEECVSLLEAGTSGWLGQLGEMLSASWRIKRTLSREVSNAVLDDLFEAIIGAGAYGAKLCGAGGGGFFLALIDPDRLSALIERVAPLSVVPIGIDVDGSTLIYRQTR

SEQ ID NO: 104 - protein, 856 aa, Methylobacterium sp.
MSLRALRSSLVASASVLGGAMPCAAQAQNAVTLGEISVVSTTPVGSGGGNSSRVQNLNGPGGLEPAGSAPPPIGPVRLRGSEQPLYKIPSTVETVTASDITVDRGTDNVVATLARRTPGLNLSDSQGNSNRPDVTYRGFTVSPVQGVPQGLAVYQNGVRINEAFGDTVNFDLIPPQAIQRIDVVTGNPVFGLNALGGAVNIQMKNGFTWQGTEISAWGGSDARTAGYVEYGKVSGPWSVYFTGDGLNDRGWRYESPSTIGRLYGDIGYRSQDSEFHLIGLAARSFFGAAAATPVDFTHRDPRAIFTYPQTTTTEVGTLQLTGRVDVSPTWDLAGNAYFRRFSQTYVDGNDGNFENCSTRSSFRGNLCFEDDGFSPAAGQTQLAFRNQFLMLGQQNQRIPFRSGIPYGTLDVTNTEATGFGGSIQAANRDRVFGLPNSFVVGGSIDAANYSFKSSSTLGVINPNLSITTDPSNPFYGNIPGLGTTQLRTAGALGIAPSSVNGSNLYMGLYTLDTLDVTDRLSLTAGARLNFARIQSEDLTGFSPDVTGTHYYNKINPVAGLTYRFTDALNLYGSYSESNRAPTPLELACSNPDRPCLLPNSLVADPPLKQVTGRTYEVGFRGALPNTYDGGVISYKIGAFRTDLSNDILSLATPGNTARAYFVNVPSTQRQGIEVGSEYTADYLRVYANYALIDATFQFNGTLSSPNNPLSDDGAIEVRKGNVVPLVPTHQFKAGFDYFITPDWQVGLYLQAFSSSYFRGDESNLNRKLPPYYVMNFQTKYQVTKNLEVFGLITNLTNNRYATFGTFAEPGAVAGNLRISDPRTTTLSQPFSVYAGIRYAFGADPVPMSAEPIIRKY

SEQ ID NO: 105 - protein, 246 aa, Methylobacterium sp.
MNAVTPAPLLAVENLRAWYGHSHILQGLSLEVRAGEIVTLVGRNGAGKTTTLKSIMGLVPKREGRVVFAGTDILARPAYERFHRGLAYVPEERRIVPGLTVEENLRLGILAARNERVSRRDEARRIEALAETFPRLKQRLKQEAITMSGGEQQMLAIARALMAEPVMVLLDEPSEGIMPILVEEMFEQFVAMKRAGTTILLVEQNVELALDISDRVYIIDGGAVVYHAPAAELRDDPDIQARYCAV

SEQ ID NO: 106 - protein, 554 aa, Methylobacterium sp.
MNFVHRYLGESRAVSDADATRLGFVPDTLREPTYFAGTVARHLPFREAISALHHVVVSDLRFKPKDRTAYFAWLQAHEQELLAEALAEKDSLRAEIEALRAEHRDIAARSDAVMRPFYDARKRYFDYLYRENFDAWIVLDPVITVHPDEVFFEAFSLDESSYGRLSCDHDTFARIGDMACGTTNIDYSHALYDEFQKIRSYRDTELAIDPTGFAVQTSGEAAYREDKIELPDSWMRGFLQVSSAMAQPAHVVDLHPVDMHAILTRLAARRERHGPRALRFLLEPDRPVSVLIEPWNERLTFRRSIYRGGEAAEIRLWGRRRLAILARTLPLARSVRLHLLGTGLPSFAVVDFGGLRFTLGLSGWTANDWSRAGQFDLLAPRGEVEADTAARVFAALRRHHAAGTGQLAAETGLDRSTVEAALGGYVQAGRAMFDLDKRVYRLRELTREPLAPGALRFASEQEAKADRFLAAGLVTLGPVEQAGDRRRLSGTVLDDGRSLTPAVELDSDDRMVGGTCQCGFYTHSRLTRGPCAHMLAVRRFFHAQVEGKPARWQA

SEQ ID NO: 107 - protein, 450 aa, Methylobacterium sp.
MADPAPITGAPEPVKAAPGAFLTPLFTDRRVAAVLGLGFAQGIPFLLVYATQSAWLVQAKVPLATIGLMSELTIAYKLKFLWAPFLDRHDAPLIGRWLGRRRGWIVATQILVALALAGVAFGDPAHWLAWTVAFSLALGVAGATQDVVIDGWRITAAPPEQQALMSSWAEIGFRIGNLAAGAGALYLSDAYGWRVAYLCMAALMAPGTVAALLAPEPPVPETPATGGFVETVRAPIRDLLARLGPLALPVLALVAGFRMPGYVSNAMAIPLFKTLGYTNTDIATVTKLFGFWIALGGTFLASAIIPRIGMMASLLIGTVTASASHLALAYLAWHGGHGGAAFWTFALTVGIDGFAYAFASIVLITYMSRLSATAHAASQYALLTSLCALPGSLLAGFSGFVIEWTGFPWFFVGTSLIGLPVALLCLLVARRHGPMEPAADAAGDAPNRAT

SEQ ID NO: 108 - protein, 246 aa, Methylobacterium sp.
MSEPAPAVTRAFVLAAGLGKRMRPVTATVPKPLVEVAGKALLDHALDRAAEAGIETAIVNVHYLPDLIEGHLARRTGGPAIQVSDERAALLETGGGIRKALPLLGDAPFVVLNSDSFWLEGPAPNLRRLIDAWDPARMDALLLVAPTATSLGYDGAGDFLMDADGRLERRGEREVAPFIYAGVAILQPGLFADTPEGAFSLNLLFDRAIAAGRLSGMRLDGQWLHVGTPDAIRAAEERVQASARAP

SEQ ID NO: 109 - protein, 202 aa, Methylobacterium sp.
MTARTIIGADPAATETSPQIHIAEEIRRRRREQGLSLETLAARSGVSRSMISKIERSEAVPSTVVLSRLAEALGVTFSRLMAPATEREILVIPASRQPILRDEASGYLRRCISPVLPGRGIDWVLNTLPPGASTGEFTAHRRGVSEYIYVLRGRLRAVIGERAVIMETGDSLYFEADAGHAFTNVGTEACEYFLVIDPSRVR

SEQ ID NO: 110 - protein, 488 aa, Methylobacterium sp.
MSDDEKPGWFGRLFGRKGAPESKSPEPVPAEDAAAEETPAEESVTPDPLSPASESEGQPVFTTAADDVARVPPAVSEPAPDDPDKNRIPDELEGADLQPEPQPPSGEAPQPDEISAESEPAAAPTETAEKRNWWSRLTGGEEETPAPAAESEPPAEVDIQPVDSAAALASDAVSGAVEGSEKQGWWSRLTAGMRRTSSALSDRVTGLFTKRKLDATTLEDLEDALIQADFGVETATRMSEAVGKGRYEKGISPDEVRAILATEIERALEPVALPIEIDSAKKPYVILTVGVNGAGKTTTIGKLSLKFKAEGRSVMLAAGDTFRAAAIEQLRVWGDRTGTPVISRAQGSDAAGLAFDAFKEARENGTDVLLIDTAGRLQNKAGLMAELEKIVRVIRKLDPEAPHATLLVLDATVGQNALSQVELFSQAAPVSGLVMTKLDGTARGGILVALATKFGLPVHFIGVGEGVEDLEPFAARDFARAITGLPKE

SEQ ID NO: 111 - protein, 522 aa, Methylobacterium sp.
MRASLTRLIPPTLARDLPASFVVFLVAMPLCMGIAMASGVPAERGLITGIIGGIVVGFLAGSPLQVSGPAAGLAVIVFEFVREHGIDALGPVLVAAGAIQLLAGALRVGGWFRAISPAVVHGMLAGIGILIVLAQIHVLTDALPKASGIDNLVAIPAAFFNFVSGPEGNRPGAVIVGLVTIAAMIGWEKIRPARLKQLPGALMGVVAGTLVAVFGDMAVKRVEVPENIFSAVTVPAMGDWSRLTEPAMIVMAITLAVIASAESLLSAAAVDRMHDGPRTQYNRELGAQGIGNVLCGLAGGLPMTGVIVRSSANVQSGAATRASTILHGSWILAFLLVLPMVLQLVPTASLAGILVVTGWRLVSPAHAVHLHERYGLATAAIWLATMVMVVATDLLTGVLTGLALSLLQVIPHFVRGPLKIEGGASETVQGGAVQAVPELRLSGSATFLQLPHLTDALERTPEGRPVRLAAEDLRHVDHTCLEMIREWAARRARSGSRIEVVGGGRSGLQHSLAMVAHAAPKD

SEQ ID NO: 112 - protein, 95 aa, Methylobacterium sp.
MIVLIAAAIVSGLATATILAPVSALAALIIAPLAASASAILACIFIAWRNTRDDVGPPDLETQADAMVAVLCEVAQQGKIVPVAAPVRVRGHRSA

SEQ ID NO: 113 - protein, 158 aa, Methylobacterium sp.
MDITGEYRIAAPRAAVWAALNDPEVLARCIPGCKELTQASPEELAAKVALKVGPVSATFAGTVRFEDIRAPEGYTLVGQGNGGMAGFAKGRAVVSLREEGADTVLTYEAKAEIGGKIASLGGRLIQGTSRKLADQFFSTFAAELGAPAPASEAAVAAP

SEQ ID NO: 114 - protein, 256 aa, Methylobacterium sp.
MSLFDLTGKTALITGSSRGIGRAIALRMAEHGARVVISSRKREACEAVVAEIEAAHGAGRAVAIPASISVKEELETLVAETESRLGPVDVLVCNAASNPYYGPLAGISDAQFRKILENNVLSNHWLIQMVAPGMVARRDGAIVIVSSIGALKGSPVIGAYNVSKAADLQLARNYAVEYGPANVRVNCLCPGLIRTDFARALWEDPEMLAATTDAAPLRRIGEPDEIAGAAVFLASAAGRFVTGQALVIDGGVTIAR

SEQ ID NO: 115 - protein, 332 aa, Methylobacterium sp.
MFIRSHLRPGGRRLAGARLATALMLATMTLATVTLGPVAARADEVVLRVGDQKGGNRSLLEIAGYAKDLPYRIAWSEFPAAAPILEALNAGALDVGYTGDLSFLTVVAAGAPIKAIGGTKSDPRTQTILVRADSPIRSAADLKGKRLAGTRGGWGQFLISATLEKAGIAPSEATFAPLNPVDAKVALMAGSVDAWAVWEPYVAFATLKDEARPIADGAGLTPTITFIVASDGAIATKRAALQDFLSRLNRARLWSLDHLDAYARNTAALTKMPEDVLRAAYTAQRTSPIALDEGVVKEMQDASDRATRYGILSKTLDVGRALDRSFTEAASN

SEQ ID NO: 116 - protein, 1029 aa, Methylobacterium sp.
MAKAKARKPETAAKSKGRAEERTDAQLSALFDAAAPAARDTRVISVRGAREHNLKNVDLTIPRDRFVVFTGLSGSGKSSLAFDTIYAEGQRRYVESLSAYARQFLEMMSKPDVDQIDGLSPAISIEQKTTSKNPRSTVGTVTEIYDYMRLLWARVGIPYSPATGEPIESQTVSQMVDRVLELPEKTRLYLLAPVVRGRKGEYRKEIAEFQKKGFQRLRIDGEYYAIDDVPKLDKKLKHDIDVVVDRIVVRDDIAARLADSFETALELADGIADIEFADAPEGEAPKKITFSSRFACPVSGFTIPEIEPRLFSFNNPFGACPTCGGIGHEMRIDPELVISDSALTLKRGAVGPWAKSTSPYYDQTLDALAKHFGFKTSVAWSALPEQAREVILFGTGKESVRFDYNDGLRSYSVNKPFEGVIPNLERRYKETESDASREEIGRFMSATPCAACDGKRLKPEALAVKIDRQDIGQVTALSVREAHRWFSEISGKLTDKQNEIAVRILKEIRDRLTFLVDVGLEYLTLARGSGSLSGGESQRIRLASQIGSGLTGVLYVLDEPSIGLHQRDNERLLGTLKRLRDLGNSVIVVEHDEDAILQADYVVDVGPGAGIHGGEIVAQGTPEELLKDPASLTAKYLTGELSVRTPKARRKPGRGMLRLVGARGHNLKNVTAEIPLGTFTCISGVSGGGKSTLIIDTLYKAAAKRLNGALEHPAPFERIEGLEHLDKVIDIDQSPIGRTPRSNPATYTGAFTPIRDWFAGLPEAKARGYQAGRFSFNVKGGRCEACSGDGVIKIEMHFLPDVYVTCDVCKGKRYDRETLEVRYRNKSIADVLDMTVEEAADLFKAVPSIREKMETLARVGLHYVRVGQQATTLSGGEAQRVKLSKELSKRATGRTLYILDEPTTGLHFHDVAKLMEVLHELVDQGNTVVVIEHNLEVIKTADWVIDMGPEGGDGGGRVVAQGTPEEIAASTASHTGRFLREVLARRPAGKAVKDAAKDSGKDAGKDAAKAKAGAASGRRSNAAGRQAAE

SEQ ID NO: 117 - protein, 226 aa, Methylobacterium sp.
VVDEAGKPVTSACTAPAFETHGLAGSISVVDRTYHYVYTDVLPEDCGLAPEKRRTGLFLRTAQDLSGPKVWSTARKLAGPLPPGTLVRVARAKGMQRWAVSYTCQRPANAPGGPVADICLQYTADMNLDGIGALKLYADPVEAGRSAAYLGLRSGGDGSGRYDRSAHFWMTDAEGNLDTPAIYPNKAGFLTWLDRLAPTASGRDASSLYGRPVYWATWTVRPIGAQ

SEQ ID NO: 118 - protein, 69 aa, Methylobacterium sp.
VPPGTPYTIEIWDRIGGKLELHLATIYPLTAAIAAFEAACLEWPTNEVTLRDRARIVRKRELPPRSATG

SEQ ID NO: 119 - protein, 212 aa, Methylobacterium sp.
VIHDRKAPITRRLVLVRHGQSVANRSGLFTGLLDSPLTEQGRIEAVAAGRRLAERSWRFSDAFTSTLTRAVVSGRLILDTLGQPGLIPQRFAALDERDYGDLSGLDKTAADARWGAERIETWRRSYAEAPPNGESLRDTVARIVPCYLRSILPAVMGGDVLVVAHGNCLRALVMALDDLSPAEVEHLELATGSVRIYEFAADTTIEARWIDG

SEQ ID NO: 120 - protein, 169 aa, Methylobacterium sp.
LDEIAALIEPQIPALRRYAVALLRDREAADDLVQDTLERALSAWSGRRRDGDLRAWLFTIERNLFLAAVRRRGRRGADLGAEALEQVPDPSADPGAALGARDVLAGLDTLPEEQRSVLLLVAVEDLSYAEAAQVLGVPLGTVMSRLSRARTRMRGFLETGRTGLLRRVK

SEQ ID NO: 121 - protein, 218 aa, Methylobacterium sp.
MVTSPIWHVVSALAVAGTLAVHGGPAHATPRHAPAGTLHFDDGGERYRGYRIEMARDVPNAEIGQLRQAAEHQVDIVEATSLDEGTKAFLRRFPVVVHSGAGERSHYSGGDHVDIAVEDPKDARPILLHESMHVYHFRRLPGGRNNPDILTFYGRAKDGGFYPAGAYLLTNQGEFFAMTASVYLHGKLAREPFTRDELRQKQPVYYRFLTRLFGPVGT

SEQ ID NO: 122 - protein, 105 aa, Methylobacterium sp.
LAETLPLRHGAYVGVGTDCTNPRNVELRTYDGGGLGSSKANDCRSRVLRQQGNVFEIEQDCRQFGGPKVEGSTERSPIRVDGPERYTDMTDGGNESFRLCPGLKP

SEQ ID NO: 123 - protein, 2106 aa, Methylobacterium sp.
VRGGLLAEGAAAAVEGLGEARGLAERPVGGATQGLDLGRDAGAGATGLGGDALQGPLDLLDPAGETPLDRAEVGVGPGHEVGERGVGGLDPLQQAAEFATHPVVGLEQLREGAARAVLYGGAQQLGRGPRLHGQFLGPRPQALGQGHAGLGERPLDRLGPVRQGPGQVHHRPVDGAAQALDMALQAVADRAAAALDAGDERAAALLDGAHQGLGALGEAAGQGLAAGIHQGAERRGTALEPGPEGLGAGVDQAGERGLAAAEGLGQRRVVGVDLLGMGPRPLVEGGDQGRRALVHQAREGLVAVVEGGGQGVGALRHQGREQPHAAVDALGEGIGAGVDATGQGLALRLHGLRQGRGAALDDAVEIPQPGVEGLDQGAGTTLDDAVEIAQPGVEGLDQSAGTASDDAVEVAQPGVEGLNQGAGATFDDAVEIPQPGVEDLHEGAGTALGRRHQGLAAVLQKPLDRRSGLLEGRPQGLDARVDALGHLAAGALDQHAEAVLHPIEQGLQLARPLLEGLGEPVLALLEQAGDGRVHPLDGRLERLGAVLDGPGDLAAAVVHEAVDLALRPLEQALKVREAGIQQVRHLGGCRVEGRAQALGPGRDGVGDVLAASVHEVAQAGLSLLEEGLQALRARVEGGGELALAGLDQRRDGGGRLLQHRAQGLDAGIDRGGEAAAAALDHAVEGRLGLLEQGAQGLGPGGEGVGEAVAAAVDQALEMAEPLVEQAAELAGPFVQGAAGGAERALQGGDQRLPAAVQRGVDRAHAAVEGGGQGAGVVVQHDADLVGPVVQQGVDRAHALVEQAADVGGAARQGVAQAALAGIEHPLGIRDGLLELCEHAVAALVDQLGEGGAAAVEQTGQLGDPAADRPEHGAALLVELARHGGDALVDETAEEVGALGEGGVHGLGAGGQSRVGALGALVHQPDHRLRTVRQRGLEAAGGLLDGAGEGADALGHGAVEAAGPIGDHRHEVAGPAGQLLVHRAGARLDEADRLEGLHAEALVHEAGALVQGLGDRLQAADDAGVEMAGALLDPASDLARVPGDVLAEAVGAALQHLHQAAMALGHDPRHLVGPGAQRLVRRAGAAAEMTVDLADEVGHGGGDRGGPLRGARLQAPQMGLDQAAGLAGPGRDRLGHGLALAGEEPVHLLQTGADALAEGGGVAADPVGQDVAAGDGLLQGGEAAAQRLVDRPDPAVGGLGEARTGLAEGVGDAGALARHGEHDVAAGAADPGLGLVGMAGEQLGDAAAMRAEGRFHLADPGGEVGRDRAGGLGEAAGGVAGPGGEFAVEVVEGGADGRDGGRAALAEFRAGAVGGVAEPGGEDRSLGLHGAGDLAGGGAQGLQGRGEPGGGIPRLRLGGLAGLIGGGAQRRGGLLGARRQGGGGIRDPALEHPHGLQGPGLDRAGGLVGRAAQGAGGRLGPALERRDRLGGAAVDLARQALDGAPQGLDRRGGPRLEALAQGREGGLQARRHRLGPVRDAAAGLLGRGCDPVLQVRGPNRHGLVEARRDAVHPLGEGASALRQPFGDPLGGADEVVAHGAGTGRQGLAGAAGRQRQGSLGIAPAALDRLGDRQAGALDLGAQREAAAGDVLGEGRAGLGEAGGDVGAHRAEALGQLRTPGQEDGFQPRHGPLEALAHGAGLALQRVADAAAGLLQGPGEGAGILGQAIGQGGAGGLEGAGRRAGAILEAAGQGLADLLDGAAHLLAGGREGVDEVAAAGGHEVDHPVAGLAQGGGDRGAAGLQGAGHPLARAGHGRHDPVRGAFQVLAQALVGAGNGAAHPVGVGDDRLALGHQLVHEGADADLVVGIGPLQGGDLAPDQGLQLAGPGERPLDAVAHRRHLAAHGLRHGQDRVGGEALGLGQTHRDLAHRTGDEAHLLGAHRQHGGDHEQHHRAEQRRGADARLQGGEPRHDGAQVALGLRPRDQHQGADPDGAGGEREQVGLGRGAHPQGLLQDADVAPVVVGHQRAVGGQEPALAARAVQARRRQVGGERRVEVEARRHGPPGGIEAAVEQRLGVTHIGQARLGAAIGAVRRQGVEVQRLLDRRERGLGRVLQLLLGRHSTPRYTGSPTPWGRLMPETQRPVYHYKTKLRQRLPIPEPSTGPRKR

SEQ ID NO: 124 - protein, 384 aa, Methylobacterium sp.
MLAMNYRGPYRVRVDRKPIPVCEHVEDAVIRVTRTCVCGSDLHLYHGMVPDTRVGTTFGHEFTGVVEEVGSGVRNLAVGDHVLVPFNIACGRCHFCKQGLFGNCHEANAQATAVGGIYGYSHTAGGYDGGQAEYVRVPYADVGPCKIPETMDLDDAVLLTDVVPTGYQAAEMGGIQRGDTVVVFGAGPVGIMAARCAWLFGAGRVIVIDHVEYRLDFARRYCPAEVYNFRSIGDPVVFLKKTTDSLGADVCIDAVGGDAAGSALHTLMGTKLKLEGGSAVALHWAINSVKKGGIVSIVGVYGPTGNLIPIGNVLNKGITIRANQASVKRLLPKLIAHIEAGRLNPKDLITHKVPLEEAADAYHLFSAKLDGCIKPVLVPPTARA

SEQ ID NO: 125 - protein, 164 aa, Methylobacterium sp.
MRNRPIDPSTVPGWGVDADPQNDPTYPMRDIARDDSRGMNWTRPPQQRARVEVLTSIEYNARPAVVGTSTPPRGVSGVIRRQAFRYSESQWAHWLMLMAADRVNVVEGVVDDLARGRVPNVPAEMGARAELAHNRSGLAGKLALTGAAIGLGILVSRLARAERL

SEQ ID NO: 126 - protein, 300 aa, Methylobacterium sp.
MPLLALPFPAIDPVAIAIGPITIKWYALAYIAGLIGGWYYARRLVMADSLWGVVKRPQVVDIDDLVVWVALGVVLGGRIGYVLFYNLPMYIADPWEILAIRNGGMSFHGGFIGAILAFVLFARGKGLNAYTLLDIGAVVVPIGLFFGRIANFVNGELWGRVAPDFRYAIVFPSGGPLPRHPSQLYEAATEGLLLFIVMAVSVRRFGFRKPGLLGGIFVLGYALARTFCEFFREPDRQLGFLFGDHLGPMGGGVTMGMLLCVPMMIVGLTYIVLAATGRTRPRHPVEAPAAEAARKAAVEA

SEQ ID NO: 127 - protein, 259 aa, Methylobacterium sp.
VTAGMFIEAPELSAHAGVRHAFFTRVGGVSEGLYASLNGGLGSQDAPERVVENRARMCAQLGLAQDRLVSLYQVHSADVVTVEAPFAAERPKADAMVTRVPGLALGIATADCGPILFADPENGVVGAAHAGWKGALTGVIGATVSAMEALGARRSRIVAVLGPTIGQASYEVGPDFVARFRSDAPGMERFLGPGTRPGHAQFDLPGFILARLGEAEIGEATALNLCTSADPERFYSYRRTTHRGEADYGRLISAITLVP

SEQ ID NO: 128 - protein, 356 aa, Methylobacterium sp.
VTPLGTEIAALIRQNGPIGVDRYMALCLGHPVHGYYRTRDPLGAQGDFTTAPEISQMFGELLGAWTAYVRGSIGSPDPLLLVELGPGRGTLMADALRALRAALSGVRVAPHLVETSPVLRAAQAHALSGTGAAWHDSIETLPEGPAIILANEFFDCLPVRQFERRPSGWHERQVGLDPAGGLAFGLSPEPVPGLAADGPDGALMSVPAAGLALIRALARRLCSEGGALLAIDYGHVRPGFGDTLQALAGHRFADPLAEPGEADLTHHVDFAALAQAARAEGAAIHGPVDQGDFLAALGLGARAERLRARASPAQAAAIDAAVARLTDPARGGMGSLFKVLAVSGPSVGPLPGFPGA

SEQ ID NO: 129 - protein, 379 aa, Methylobacterium sp.
MGPGREAVDAAALLDGFSACLSGLGLPLARATTHAPTLHPSFRWVMRVWHPGTSSLALRRRHGIEGTPTFHGNTVEHVVETRTPLQCRLDGDGPLPFPVLGELRNEGLTDYLIAPLRAARGRMGAASWATARPGGFTPIEIETLLALVEPFSLLFEIKALDDMLGAVLSAYVGRDPARQILAGTVRRGDVRLMRAAMMLTDLRGFGELSDRQSPDHVVAALNRMFDAIVPAVEAEGGEVLKYIGDGLLAVFDADRDEAEARRAALRAAEAALDALATLRDGDRAAFEVGVALHVGEVAYGNIGGGDRVDFTAIGRDLNVLARVERLCKTYDTPLIATDTFLHGLAHALEPLGIVALRGFAERHALFGCRRTAPVEAPAV

SEQ ID NO: 130 - protein, 327 aa, Methylobacterium sp.
MTHVRSSLRALLAGAALLLAQGQPGSAAGFDGAIKNNALALNAAGTVAAVSNSEESAVIVYDVAKGTVLRRLDGFVTPRNIVFAPDGTRFYVSDSGTGRITIYETATGKEVGILAAGPGAFGTVLSADGGKLYVNNEAASTLTVFDTKTMLAEAVIPGFAQPRQGVKLSPDGKTVFVTNFLGDKITLVDTATNKITGEIAGFDKLRAISITKDGKTLFAANSGRNTVGVVDVAARKVTSEVTVGKDPYGAALTPDGRFVYSGNLKDNSLSVIDTGTLTVVATVTGLNEPRQAIAFSTDNARAYVLNRDLSVAVVDRAKNAVVSTMKP

SEQ ID NO: 131 - protein, 66 aa, Methylobacterium sp.
VLAVWPPKVLSLTLIASFSGAVWRDRAIRARLAHAEIPMMPRLLTVGIAALPIAISVLAATALLRA

SEQ ID NO: 132 - protein, 381 aa, Methylobacterium sp.
MRVAIVHYWLIGMRGGEKVVEALCDLYPEADIFTHAYAPQSMSPTIRAHRVRTSFIGRLPFATSRYKSYLPLMPMALEQLDLRGYDLIISSESGPAKGIIPPSDALHICYCHSPMRYVWNMYHDYRERTGLLTRLLMPPVAHYVRNWDAVSAGRVHEFIANSDTVARRIETYYRRQAKVIHPPVDTAAFEIAPDGQRGDYHLMVGEMVRYKRPELAIQAFNRLEQPLVVIGGGEMLRELRSMAGPHIKILGPQPFEVLKHHYARCQALIFPGEEDFGIVPVEAMASGRPVVAFGKGGVTETVIDGVTGTFFHEQSVDALIDAVQRCRAIGVEPERLVRRAADFGVGRFADEISCFVDGVLARERLAAPRPPREPSRAYLVQ

SEQ ID NO: 133 - protein, 511 aa, Methylobacterium sp.
VASPVAVTEIQPKAQARPAPQAALSPGEIRAIVYGLMTAMLLAALDQTIVATAMPTIGLDLGDAANLPWIVTAYLLASTAVTPLYGKLSDIHGRRVMLLIAIATFVVGSLACALAPTMVALALARGLQGVGGGGLIALAQTILADIMSPKERARYQVVIAGVFVTASVAGPLLGGLFAQHLHWSLIFWINLPIGVLAFALTNANLKRLPRHERRHRLDYPGAALMVAGSVTLLLALSWGGVRYPWDSAPVLALLAGAAVLSGAFAARLATAAEPLIPTEVLKDRVVYSATLAACFAMGTFIGLTIYVPIFLEGVIGLSASESGVALVPLMIGTVTGATLSGRSMLHFRHYKRLPLAMMCVSLACCATIAWQGRALPFWLMEVLFALLSMGIGTILPLSTISIQNAVETHQLGIATAAMNFFRSLGGALIVAAFGTIVLGGAAGGAGGGAHDVESLIRGADPAQLALTFRHVFLAACLGLLGAFTFLALMEERPLRERTSPRMAAETPEASA

SEQ ID NO: 134 - protein, 308 aa, Methylobacterium sp.
MTVAILLSGQGGQHPAMFNLTADHPAAQDVFAAARPLLGGADPRDLVRAGGNNLHANRTGQILCCVAALAAWRALAEAEPDRAIVAGYSIGDLAAWGVAGRLDPADVLALAARRAEAMDAASGSGFGLAGIRGLSLDALGDLAARHGSHLAIRNAADSGVVGGSRAALEALCREATASGAQRAVVLPVHTPSHTPLLDAATAAFRDALAAVSSLRPPPRAPRLISGLDGSTVFRDADGREKLALQISRTIDWAACLEACREYGADRVLELGPGHALATMARAALPAARVHAVEEFRSLGGVADWLARP

SEQ ID NO: 135 - protein, 770 aa, Methylobacterium sp.
MNPAMSGPAPDPGRLTVIGVRHHSPACAGLVRRTIAALRPACVLIEGPVDFNPHLPDLALGHDLPVAIFSFRADAAGSAASYTPFCAFSPEWQALEAGRAVGAETLFCDLPAWDPAFGRRANRYADLHGARAEAAERALAAALGVADQDALWDVLAEAAPEAELPARLDRYFALLRPPGTDDLAEEARERFMGAYAAHALRAAGDRPVVLVCGGWHADAVRRHAARADGTRPEPAPPEPDLRTGSYVVPYAYPRLDRFSGYAAGMPAPGYYERVAESGLAPAADWAMTAITAALREAGQVVSTADRVAWRVHAEALARLRAHPAILRTDLIDAALAALVKDALDRPPAWAAGGAAPGHPALAAMLRALTGPREGRLAPGTRQPPLVADVAERLRAADLEPGPARRSVDLDWAEPGDRARAHLLHRLVLLGLPGIAREGPDRAGPGLPRERFTLARHPHWLGALIEASLWGGTLEMAASARITARVEAAPDSLGILTGALSDALFAGLTLEGELLARLSAGIAAAHDATALGAAGAGIVRLYRFGDAFAPARPALARLCAVLAARALFVVEAVPDPRAGLGVIPLVLACRDLFREVGAEIPGLDDLRAPFSAMLGRRLADPETPPALAGAALGFRVACGAAGSDPEAALAWLRRFGLPATLGDFLAGLFALAREEIAADATLASVEGLVAAWGDDDFLRALPSLRMAFAWFPPRERERIAVAILRRSGLGEARAEVEALAWMRQRARPADQAEALAREARVAARLARHGLT